(19) United States
(12) Patent Application Publication
(10) Pub. No.: US 2017/0354639 A1
(43) Pub. Date: Dec. 14, 2017

(54) DITERPENOID DERIVATIVES AND METHODS OF USE THEREOF

(71) Applicant: Biogen MA Inc., Cambridge, MA (US)

(72) Inventors: Jianhua Chao, Cambridge, MA (US); Brian Lucas, Arlington, MA (US)

(21) Appl. No.: 15/521,239

(22) PCT Filed: Oct. 23, 2015
(86) PCT No.: PCT/US2015/057121

§ 371 Date: Apr. 21, 2017

Related U.S. Application Data
(60) Provisional application No. 62/068,447, filed on Oct. 24, 2014.

(51) Int. Cl. A61K 31/365; C07D 413/06; A61K 31/423; C07D 407/06; C07D 411/06; A61K 31/39; C07D 307/60

(52) U.S. Cl. CPC A61K 31/365; A61K 31/39; C07D 413/06; C07D 307/60; C07D 407/06; C07D 411/06; A61K 31/423

(57) ABSTRACT

Provided herein are compounds of formula (I) and compositions containing the compounds. The compounds and compositions are useful in the methods of treating, amelioration or prophylaxix of diseases associated with Nrf2/NF-_{κ}B pathways. The diseases associated include, but are not limited to a fibrotic disease such as lung fibrosis, liver fibrosis, kidney fibrosis, and scleroderma, or a neurodegenerative disease, such as multiple sclerosis, amyotrophic lateral sclerosis, Parkinson's disease, Huntington's disease, and Alzheimer's disease, and sickle cell disease.

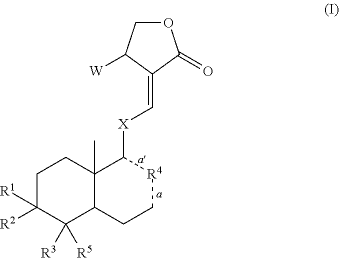

### RELATED APPLICATIONS

This application claims priority to U.S. provisional application No. 62/068,447, filed Oct. 24, 2014. The disclosure of the above referenced application is incorporated by reference herein in its entirety.

### FIELD

Compounds that are diterpenoid derivatives, compositions comprising the compounds, and methods for treatment using the compounds are provided.

### BACKGROUND

Nrf2 (Nuclear factor E2-related factor 2) is a master transcription factor of oxidative and xenobiotic stress responses. It regulates through antioxidant response element (ARE) the expression of many Phase II detoxifying enzymes and antioxidative proteins including NQO-1, HO-1, GCLC, GST, NAT, SRXN etc. Over two hundred Nrf2/ARE dependent genes are exploited for antioxidant defense. Activation of the Nrf2/ARE pathway is the primary cellular defense mechanism for responding to a wide variety of potentially toxic stimuli (including inflammatory, electrophilic, and oxidative stress). It is cytoprotective and antioxidative.

Nrf2 is expressed ubiquitously in a range of tissues and, under normal basal conditions, is sequestered in the cytoplasm in a complex with Keap1 protein. However, when cells are under oxidative stress and overloaded with reactive oxygen or nitrogen species (ROS or RNS), or electrophilic entities, Nrf2 rapidly translocates to the nucleus, forms heterodimer with small protein Maf, then binds to the antioxidant response element, resulting in increased transcription of many of the aforementioned antioxidant and detoxifying genes including NQO-1, HO-1, and SRXN1. Knocking down Nrf2 in mice leads to increased sensitivity to many chemically induced toxicities and disease pathologies. See, e.g., Ma, Annu Rev Pharmacol Toxicol 2013; 53:401-26; Nguyen et al., J Bio Chem 2009; 284: 13291-13295; Nguyen et al., Annu Rev Pharmacol Toxicol 2003; 43:233-260; McMahon et al., Cancer Res 2001; 61:3299-3307.

Oxidative stress is caused by increased production/insufficient catabolism of reactive oxygen species (ROS). It promotes activation of inflammatory signaling mechanisms, causes generation of covalently modified proteins, lipids and nucleic acids with pro-inflammatory and antigenic properties. Sustained oxidative stress is a major contributor to cell death and tissue damage, and has been implicated in the pathogenesis of a variety of diseases including a) neurodegenerative disease such as multiple sclerosis (MS), amyotrophic lateral sclerosis (ALS), Alzheimer's disease, and Parkinson's disease, and b) fibrotic disease such as lung, liver, kidney and scleroderma, as well as c) Sickle cell disease (SCD).

Activation of Nrf2 could reduce the oxidative stress and has promising clinical potential in many of these diseases. For neurodegenerative diseases, see, e.g., Sandberg et al., Neuropharmacology, 2014, 79:298-306; van Muiswinkel et al., Curr. Drug Targets CNS Neurol. Disord, 2005; 4:267 281; Burton N. C. et al., Comprehensive Toxicology, 2010, 59-69; Jazwa et al., Curr Drug Targets, 2010, 11:1517-1531; for Sickle cell disease, see, e.g. Belcher et al., J Clin Invest, 2006, 116:808-816; Macari et al., Blood, 2011, 117:5987-5997; and for fibrotic and immunological diseases, see, e.g. Impellizzeri et al., Pharmacological Research, 2014, 81:91-102; Kikuchi et al., Respiratory Research, 2010, 11:31; Gao et al., Clinical Pharmacology: Advances and Applications, 2014, 6:19-34; Kanninena et al., PNAS, 2009, vol. 106, no. 38: 16505-16510; Barone et al. Disease Models & Mechanisms, 2011, 4: 701-707; Ramsey et al. J Neuropathol Exp Neurol. 2007, 66(1): 75-85; Jin et al., PLOS ONE, 2013, vol. 8 (3):| e57932; Vargas et al., The Journal of Neuroscience, 2008, 28(50):13574-13581; and Vargas et al., PLOS ONE, 2013, vol. 8 (2): e56625.

The nuclear factor-kappa B (NF-κB) is a widely expressed, pleiotropic transcription factor that has been implicated in many cellular processes including inflammation. There may be a tangible link between Nrf2 and NF-κB pathways as discussed by Impellizzeri et al. (Pharmacological Research, 2014, 81:91-102) that activation of Nrf2 leads to the transcription of antioxidant genes that are involved in the elimination and/or removal of ROS and inhibition of NF-κB. In addition, in the lungs of Nrf2 knock-out mice, the protein levels of the inflammatory cytokines TNF-α and MIP-2 are elevated, and the nuclear location of NF-κB is also elevated comparing to wild type mice. This evidence suggests NF-κB is activated in the lungs of mice where Nrf2 is absent (Kikuchi et al., Respiratory Research, 2010, 11:31). M. Buelna-Chontal et al. report in Cellular Signalling, 2013, 25: 2548-2557, that NF-κB pathway is inhibited by several Nrf2 activators. Also, see Li et al. Biochem. Pharmacol., 2008, 76: 1485-1489, Kumar et al. Eur. J. Pharmacol., 2013, 700: 32-41 and Tobón-Velasco et al., Toxicology 2013, 304: 109-119.

TECFIDERA™ was recently approved by the U.S. Food and Drug Administration for the treatment of subjects with relapsing forms of multiple sclerosis. TECFIDERA™ contains dimethyl fumarate (DMF). Preclinical and clinical data suggest dimethyl fumarate (DMF) has beneficial effects on neuroinflammation, neurodegeneration, and toxic-oxidative stress. See, e.g., Linker R. A., et al., Brain 2011; 134:678-92 and Scannevin R. H., et al., J Pharmacol Exp Ther, 2012, 341:274-284. The beneficial effects of DMF and its primary metabolite, monomethyl fumarate (MMF), appear to be mediated, at least in part, through activation of the Nrf2 antioxidant response pathway. DMF also exhibits inhibitions to adhesion molecules and cytokines, providing potential anti-inflammatory properties (Nguyen et al., J Bio Chem 2009; 284: 13291-13295). The inhibition of inflammatory cytokines may derive from the modulation of the NF-κB signaling pathway independently, or via the activation of the Nrf2 pathway which in turn influences the NF-κB pathway.

While DMF is efficacious in the treatment of relapsing forms of multiple sclerosis, there is a need to find improved Nrf2 activators which will combine anti-oxidative and anti-inflammatory activities to be applied to a broad spectrum of diseases that in need of effective intervention.

### SUMMARY

Provided herein are compounds that, in certain embodiments, activate Nrf2 pathway, pharmaceutical compositions containing the compounds and methods of use thereof. In one embodiment, the compounds for use in the compositions and methods provided herein are of Formula I:

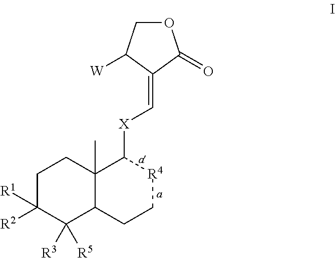

or a single stereoisomer, a mixture of stereoisomers, a racemic mixture of stereoisomers, a solvate, a hydrate, or a pharmaceutically acceptable salt thereof, wherein:

R¹, R², R³ and R⁵ are selected as follows:

i) R¹ and R² are each independently H, C₁₋₆alkyl, OR¹⁰, or NR^{11a}R^{11b}; and R³ and R⁵ are each independently H or C₁₋₆alkyl; provided that R¹ and R² are not both OR¹⁰ or NR^{11a}R^{11b} at the same time; or

ii) R² and R³ together with the carbon atoms on which they are substituted form ring b, where ring b is a 4 to 6 membered optionally substituted carbocyclic, heterocyclic or heteroaryl ring; and R¹ and R⁵ are each independently H and C₁₋₆ alkyl;

R¹⁰, R^{11a} and R^{11b} are each independently H, C₁₋₆ alkyl, or 4 to 6 membered optionally substituted carbocyclic or heterocyclyc ring;

R⁴, bond a and bond a′ are selected as follows:

i) R⁴ is CR⁶, bond a is a double bond, and bond a′ is a single bond; or

ii) R⁴ is CR⁶, bond a is a single bond, and bond a′ is a double bond; or

iii) R⁴ is C═CH₂ or CR⁶R⁷, and bonds a and a′ are single bonds;

R⁶ and R⁷ are each independently H or C₁₋₆alkyl; or R⁶ and R⁷ together with the carbon atom on which they are substituted form a 3-6 membered optionally substituted carbocyclic ring;

W is OH or NHR⁹;

R⁹ is C(═O)R¹² or SO₂R^{12a};

R¹² is H, C₁₋₆alkyl or OR^{12a};

R^{12a} is C₁₋₆alkyl; and

X is straight or branched C₁₋₆alkylene, optionally with one or two oxygen atoms in the chain;

where the substituents on the carbocyclic and heterocyclic rings, and on the alkyl groups for R¹, R², R³, R⁵, R⁶, R⁷, R⁹, R¹⁰, R¹¹, R^{11a} and R¹², when present are one to three groups Q¹, where Q¹ is C₁₋₆alkyl, hydroxy, oxo, amino, halo, C₁₋₆alkoxy, hydroxy C₁₋₆alkyl, haloC₁₋₆alkyl, aminoC₁₋₆alkyl, C₁₋₆alkoxy C₁₋₆alkyl, and C₃₋₆cycloalkyl;

and the compound is selected such that

i) when W is OH, R⁴ is C═CH₂ or CH—CH₃, one of R¹ or R² is OH and the other is H, and one of R³ and R⁵ is hydroxymethyl, then the other of R³ or R⁵ is H;

ii) when W is OH, R⁴ is C═CH₂, one of R³ or R⁵ is CH₃ and the other is hydrogen, and one of R¹ and R² is OH, then the other of R¹ or R² is alkyl;

iii) when W is OH, R⁴ is C═CH₂, at least one of R¹ or R² is OH, and one of R³ and R⁵ is aminoalkyl, then the other of R³ or R⁵ is H;

iv) when W is OH, R⁴ is C═CH₂, R¹ and R² are both H, then at least one of R³ and R⁵ is other than methyl;

v) when W is OH, R⁴ is C═CH₂, and R² and R³ together with the carbon atoms on which they are substituted form a 4 membered heterocyclic ring having one oxygen atom; then R¹ is not H;

vi) when W is OH, R⁴ is C—CH₃, and bond ring b is a five membered ring containing two heteroatoms, then at least one heteroatom in ring b is other than nitrogen; and

vii) when ring b is a six membered heterocyclic ring containing two oxygen atoms, then ring b contains at least one additional heteroatom.

Pharmaceutical compositions containing a compound of Formula I and a pharmaceutically acceptable carrier are provided herein.

Also provided is a method of activating the Nrf2 pathway, comprising contacting a cell with a sufficient amount of a compound of formula I described herein.

Also provided is a method of treating, prophylaxis, managing or amelioration of a disease (e.g., a neurodegenerative disease), comprising administering to a subject in need of treatment for the disease an effective amount of a compound of formula I described herein.

### DETAILED DESCRIPTION

Definitions

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

As used herein “subject” is an animal, such as a mammal, including human, as a patient.

As used herein, pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamineand other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates.

As used herein, treatment means any manner in which one or more of the symptoms of a disease or disorder are ameliorated or otherwise beneficially altered.

As used herein, amelioration of the symptoms of a particular disorder by administration of a particular compound or pharmaceutical composition refers to any lessening, whether permanent or temporary, lasting or transient that can be attributed to or associated with administration of the compound or composition.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

The terms “therapeutically effective dose” and “therapeutically effective amount” refer to that amount of a compound which results in prevention or delay of onset or amelioration of symptoms of a disease in a subject or an attainment of a desired biological outcome, such as reduced neurodegeneration (e.g., demyelination, axonal loss, and neuronal death), reduced inflammation of the cells of the CNS, or reduced tissue injury caused by oxidative stress and/or inflammation in a variety of cells.

As used herein, the IC₅₀ refers to an amount, concentration or dosage of a particular test compound that achieves a 50% inhibition of a maximal response in an assay that measures such response. The EC₅₀ refers to an amount, concentration or dosage of a particular test compound that achieves a 50% inhibition of a maximal response or 50% activation of a maximal response in an assay.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. As such, one of skill in the art will recognize that administration of a compound in its (R) form is equivalent, for compounds that undergo epimerization in vivo, to administration of the compound in its (S) form.

As used herein, the nomenclature alkyl, alkoxy, carbonyl, etc. is used as is generally understood by those of skill in this art.

As used herein, alkyl, alkenyl and alkynyl carbon chains, if not specified, contain from 1 to 20 carbons, or 1 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, ethene, propene, butene, pentene, acetylene and hexyne. As used herein, lower alkyl, lower alkenyl, and lower alkynyl refer to carbon chains having from about 1 or about 2 carbons up to about 6 carbons.

As used herein, “cycloalkyl” refers to a saturated mono- or multicyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion.

As used herein, “substituted alkyl,” “substituted alkenyl,” “substituted alkynyl,” “substituted cycloalkyl,” “substituted cycloalkenyl,” and “substitued cycloalkynyl” refer to alkyl, alkenyl, alkynyl, cycloalkyl, cycloalkenyl and cycloalkynyl groups, respectively, that are substituted with one or more substituents, in certain embodiments one to three or four substituents, where the substituents are as defined herein.

As used herein, “aryl” refers to aromatic monocyclic or multicyclic groups containing from 6 to 19 carbon atoms. Aryl groups include, but are not limited to groups such as fluorenyl, substituted fluorenyl, phenyl, substituted phenyl, naphthyl and substituted naphthyl, wherein the substituents, when present, are one or more substituents as defined herein.

As used herein, “heteroaryl” refers to a monocyclic or multicyclic aromatic ring system, in certain embodiments, of about 5 to about 15 members where one or more, in one embodiment, 1 to 3 of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrrolidinyl, pyrimidinyl, tetrazolyl, thienyl, pyridyl, pyrrolyl, N-methylpyrrolyl, quinolinyl and isoquinolinyl. Unless otherwise specified, heteroaryl groups are optionally substituted.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3 of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

A heterocyclic ring can be attached to its pendant group at any heteroatom or carbon atom that results in a stable structure and any of the ring atoms can be optionally substituted. Examples of such saturated or partially unsaturated heterocyclic radicals include, but are not limited to, tetrahydrofuranyl, tetrahydrothiophenyl pyrrolidinyl, piperidinyl, pyrrolinyl, oxazolidinyl, piperazinyl, dioxanyl, dioxolanyl, diazepinyl, oxazepinyl, thiazepinyl, and morpholinyl.

As used herein, “substituted aryl,” “substituted heteroaryl” and “substituted heterocyclyl” refer to aryl, heteroaryl and heterocyclyl groups, respectively, that are substituted with one or more substituents, in certain embodiments one to three or four substituents, where the substituents are as defined herein.

As used herein, “halo”, “halogen” or “halide” refers to F, Cl, Br or I.

As used herein, “alkoxy” refers to RO, in which R is alkyl, including lower alkyl.

As used herein, “aryloxy” refers to RO—, in which R is aryl, including lower aryl, such as phenyl.

As used herein, “Amine” or “amino” refers to a group having the formula —NR′R″ wherein R′ and R″ are each independently hydrogen, alkyl, haloalkyl, hydroxyalkyl or alkoxyalkyl or wherein R′ and R″, together with the nitrogen atom to which they are attached form a heterocyclyl optionally substituted with halo, oxo, hydroxy or alkoxy.

As used herein, “aminoalkyl” refers to an alkyl group in which one or more of the hydrogen atoms are replaced by amino. Such groups include, but are not limited to, —CH₂NH₂, —CH₂NH(CH₃) and —CH₂N(CH₃)₂.

Where the number of any given substituent is not specified (e.g., “haloalkyl”), there may be one or more substituents present. For example, “haloalkyl” may include one or more of the same or different halogens.

As described herein, the compounds provided herein may, when specified, contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent a specified group, the substituent may be either the same or different at every position. Combinations of substituents encompass those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Examples of optionally substituted groups that is, optional substituents include halogen, —NO₂, —CN, —OR, —SR, —N(R)₂, —C(O)R, —CO₂R, —N(R)C(O)OR, —C(O)N(R)₂, —OC(O)R, —N(R)C(O)R, —S(O)R, —S(O)₂R, or —S(O)₂N(R)₂. Each R is independently hydrogen or C₁₋₆ aliphatic; or two R groups attached to the same nitrogen are taken together with their intervening atoms to form an optionally substituted 3-7 membered saturated or partially unsaturated monocyclic heterocyclic ring having 1-2 heteroatoms, independently nitrogen, oxygen, or sulfur. Optionally substituted groups of aliphatic can further include, but are not limited to, phenyl, 3-7 membered saturated or partially unsaturated monocyclic carbocyclic ring, 3-7 membered saturated or partially unsaturated monocyclic heterocyclic ring having 1-3 heteroatoms independently nitrogen, oxygen, or sulfur, or a 5-6 membered heteroaryl ring having 1-3 heteroatoms independently nitrogen, oxygen, or sulfur. For example, (cycloalkyl)alkyl, (cycloalkenyl)alkyl or (cycloalkyl)alkenyl, heterocyclylalkyl. Optionally substituted groups of phenyl, heterocycle, carbocycle, and heteroaryl can further include optionally substituted aliphatic groups.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

Compounds

In certain embodiments, the compounds for use in the compositions and methods provided herein are of Formula I:

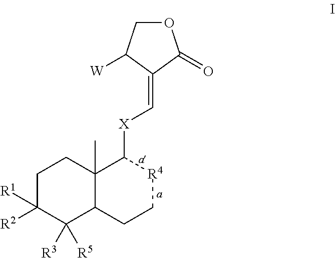

or a single stereoisomer, a mixture of stereoisomers, a racemic mixture of stereoisomers, a solvate, a hydrate, or a pharmaceutically acceptable salt thereof, wherein:

R¹, R², R³ and R⁵ are selected as follows:

i) R¹ and R² are each independently H, C₁₋₆alkyl, OR¹⁰, or NR^{11a}R^{11b}; and R³

and R⁵ are each independently H, hydroxyC₁₋₆alkyl and C₁₋₆alkyl; provided that R¹ and R² are not both OR¹⁰ or NR^{11a}R^{11b} at the same time; or

ii) R² and R³ together with the carbon atoms on which they are substituted form ring b, where ring b is a 4 to 6 membered optionally substituted carbocyclic, heterocyclic or heteroaryl ring; and R¹ and R⁵ are each independently H and C₁₋₆ alkyl;

R¹⁰, R^{11a} and R^{11b} are each independently H, C₁₋₆ alkyl, or 4 to 6 membered optionally substituted carbocyclic or heterocyclyc ring;

R⁴, bond a and bond a′ are selected as follows:

i) R⁴ is CR⁶, bond a is a double bond, and bond a′ is a single bond; or

ii) R⁴ is CR⁶, bond a is a single bond, and bond a′ is a double bond; or

iii) R⁴ is C═CH₂ or CR⁶R⁷, and bonds a and a′ are single bonds;

R⁶ and R⁷ are each independently H or C₁₋₆alkyl; or R⁶ and R⁷ together with the carbon atom on which they are substituted form a 3-6 membered optionally substituted carbocyclic ring;

W is OH or NHR⁹;

R⁹ is C(═O)R¹² or SO₂R^{12a};

R¹² is H, C₁₋₆alkyl or OR^{12a};

R^{12a} is C₁₋₆alkyl; and

X is straight or branched C₁₋₆alkylene, optionally with one or two oxygen atoms in the chain;

where the substituents on the carbocyclic and heterocyclic rings, and on the alkyl groups for R¹, R², R³, R⁵, R⁶, R⁷, R⁹, R¹⁰, R¹¹, R^{11a} and R¹², when present are one to three groups Q¹, where Q¹ is C₁₋₆alkyl, hydroxy, oxo, amino, halo, C₁₋₆alkoxy, hydroxy C₁₋₆alkyl, haloC₁₋₆alkyl, aminoC₁₋₆alkyl, C₁₋₆alkoxy C₁₋₆alkyl, and C₃₋₆cycloalkyl;

and the compound is selected such that

i) when W is OH, R⁴ is C═CH₂ or CH—CH₃, one of R¹ or R² is OH and the other is H, and one of R³ and R⁵ is hydroxymethyl, then the other of R³ or R⁵ is H;

ii) when W is OH, R⁴ is C═CH₂, one of R³ or R⁵ is CH₃ and the other is hydrogen, and one of R¹ and R² is OH, then the other of R¹ or R² is alkyl;

iii) when W is OH, R⁴ is C═CH₂, at least one of R¹ or R² is OH, and one of R³ and R⁵ is aminoalkyl, then the other of R³ or R⁵ is H;

iv) when W is OH, R⁴ is C═CH₂, R¹ and R² are both H, then at least one of R³ and R⁵ is other than methyl;

v) when W is OH, R⁴ is C═CH₂, and R² and R³ together with the carbon atoms on which they are substituted form a 4 membered heterocyclic ring having one oxygen atom; then R¹ is not H;

vi) when W is OH, R⁴ is C—CH₃, and bond ring b is a five membered ring containing two heteroatoms, then at least one heteroatom in ring b is other than nitrogen; and

vii) when W is OH, and ring b is a six membered heterocyclic ring containing two oxygen atoms, then ring b contains at least one additional heteroatom.

In one embodiment, the compounds provided herein are of formula II:

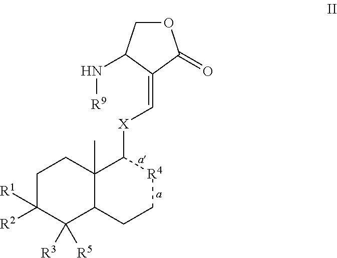

or a single stereoisomer, a mixture of stereoisomers, a racemic mixture of stereoisomers, a solvate, a hydrate, or a pharmaceutically acceptable salt thereof, wherein:

R¹, R², R³ and R⁵ are selected as follows:

i) R¹ and R² are each independently H, C₁₋₆alkyl, OR¹⁰, or NR^{11a}R^{11b}; and R³

and R⁵ are each independently H, hydroxyC₁₋₆alkyl and C₁₋₆alkyl; provided that R¹ and R² are not both OR¹⁰ or NR^{11a}R^{11b} at the same time; or

ii) R² and R³ together with the carbon atoms on which they are substituted form ring b, where ring b is a 4 to 6 membered optionally substituted carbocyclic, heterocyclic or heteroaryl ring; and R¹ and R⁵ are each independently H and C₁₋₆ alkyl;

R¹⁰, R^{11a} and R^{11b} are each independently H, C₁₋₆ alkyl, or 4 to 6 membered optionally substituted carbocyclic or heterocyclyc ring;

R⁴, bond a and bond a′ are selected as follows:

i) R⁴ is CR⁶, bond a is a double bond, and bond a′ is a single bond; or

ii) R⁴ is CR⁶, bond a is a single bond, and bond a′ is a double bond; or

iii) R⁴ is C═CH₂ or CR⁶R⁷, and bonds a and a′ are single bonds;

R⁶ and R⁷ are each independently H or C₁₋₆alkyl; or R⁶ and R⁷ together with the carbon atom on which they are substituted form a 3-6 membered optionally substituted carbocyclic ring;

R⁹ is C(═O)R¹² or SO₂R^{12a};

R¹² is H, C₁₋₆alkyl or OR^{12a};

R^{12a} is C₁₋₆alkyl; and

X is straight or branched C₁₋₆alkylene, optionally with one or two oxygen atoms in the chain; and

where the substituents on the carbocyclic and heterocyclic rings, and on the alkyl groups for R¹, R², R³, R⁵, R⁶, R⁷, R⁹, R¹⁰, R¹¹, R^{11a} and R¹², when present are one to three groups Q¹, where Q¹ is C₁₋₆alkyl, hydroxy, oxo, amino, halo, C₁₋₆alkoxy, hydroxy C₁₋₆alkyl, haloC₁₋₆alkyl, aminoC₁₋₆alkyl, C₁₋₆alkoxy C₁₋₆alkyl, and C₃₋₆cycloalkyl.

In one embodiment, the compounds provided herein are of formula III:

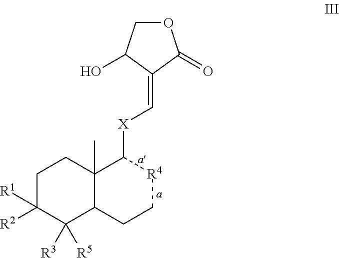

or a single stereoisomer, a mixture of stereoisomers, a racemic mixture of stereoisomers, a solvate, a hydrate, or a pharmaceutically acceptable salt thereof, wherein:

R^{l}, R², R³ and R⁵ are selected as follows:

i) R¹ and R² are each independently H, C₁₋₆alkyl, OR¹⁰, or NR^{11a}R^{11b}; and R³

and R⁵ are each independently H, hydroxyC₁₋₆alkyl and C₁₋₆alkyl; provided that R¹ and R² are not both OR¹⁰ or NR^{11a}R^{11b} at the same time; or

ii) R² and R³ together with the carbon atoms on which they are substituted form ring b, where ring b is a 4 to 6 membered optionally substituted carbocyclic, heterocyclic or heteroaryl ring; and R^{l} and R⁵ are each independently H and C₁₋₆ alkyl;

R¹⁰, R^{11a} and R^{11b} are each independently H, C₁₋₆ alkyl, or 4 to 6 membered optionally substituted carbocyclic or heterocyclyc ring;

R⁴, bond a and bond a′ are selected as follows:

i) R⁴ is CR⁶, bond a is a double bond, and bond a′ is a single bond; or

ii) R⁴ is CR⁶, bond a is a single bond, and bond a′ is a double bond; or

iii) R⁴ is C═CH₂ or CR⁶R⁷, and bonds a and a′ are single bonds;

R⁶ and R⁷ are each independently H or C₁₋₆alkyl; or R⁶ and R⁷ together with the carbon atom on which they are substituted form a 3-6 membered optionally substituted carbocyclic ring;

X is straight or branched C₁₋₆alkylene, optionally with one or two oxygen atoms in the chain; and

where the substituents on the carbocyclic and heterocyclic rings, and on the alkyl groups for R¹, R², R³, R⁵, R⁶, R⁷, R¹⁰, R¹¹, and R^{11a}, when present are one to three groups Q¹, where Q¹ is C₁₋₆alkyl, hydroxy, oxo, amino, halo, C₁₋₆alkoxy, hydroxy C₁₋₆alkyl, haloC₁₋₆alkyl, aminoC₁₋₆alkyl, C₁₋₆alkoxy C₁₋₆alkyl, and C₃₋₆cycloalkyl, wherein the compound is selected such that

i) when R⁴ is C═CH₂ or CH—CH₃, one of R¹ or R² is OH and the other is H, and one of R³ and R⁵ is hydroxymethyl, then the other of R³ or R⁵ is H;

ii) when R⁴ is C═CH₂, one of R³ or R⁵ is CH₃ and the other is hydrogen, and one of R¹ and R² is OH, then the other of R¹ or R² is alkyl;

iii) when R⁴ is C═CH₂, at least one of R¹ or R² is OH, and one of R³ and R⁵ is aminoalkyl, then the other of R³ or R⁵ is H;

iv) when R⁴ is C═CH₂, R¹ and R² are both H, then at least one of R³ and R⁵ is other than methyl;

v) when R⁴ is C═CH₂, and R² and R³ together with the carbon atoms on which they are substituted form a 4 membered heterocyclic ring having one oxygen atom; then R¹ is not H;

vi) when R⁴ is C—CH₃, and bond ring b is a five membered ring containing two heteroatoms, then at least one heteroatom in ring b is other than nitrogen; and

vii) when ring b is a six membered heterocyclic ring containing two oxygen atoms, then ring b contains at least one additional heteroatom.

In one embodiment, the compounds of formula I, II or III are selected such that R⁴ is C═CH₂ or CR⁶R⁷, bonds a and a′ are single bonds; and R⁶ and R⁷ are each independently H or C₁₋₆alkyl; or R⁶ and R⁷ together with the carbon atom on which they are substituted form a 3-6 membered optionally substituted carbocyclic ring.

In one embodiment, the compounds of formula I, II or III are selected such that R⁴ is C═CH₂ and bonds a and a′ are single bonds.

In one embodiment, the compounds of formula I, II or III are selected such that R⁴ is CR⁶R⁷, bonds a and a′ are single bonds; and R⁶ and R⁷ together with the carbon atom on which they are substituted form a 3 membered carbocyclic ring.

In one embodiment, the compounds of formula I, II or III are selected such that R¹, R², R³ and R⁵ are selected as follows:

i) R¹ and R² are each independently H, C₁₋₆alkyl, OR¹⁰, or NR^{11a}R^{11b}; and R³ and R⁵ are each independently H, hydroxyalkyl or C₁₋₆alkyl; provided that R¹ and R² are not both OR¹⁰ or NR^{11a}R^{11b} at the same time; or

ii) R² and R³ together with the carbon atoms on which they are substituted form ring b, where ring b is a 4 to 6 membered carbocyclic, heterocyclic or heteroaryl ring, ring b is optionally substituted with oxo; and R¹ and R⁵ are each independently H and C₁₋₆ alkyl;

R¹⁰, R^{11a} and R^{11b} are each independently H or C₁₋₆ alkyl;

W is OH or NHC(═O)R¹²;

R¹² is H or C₁₋₆alkyl;

X is straight C₁₋₂alkylene;

R⁴ is C═CH₂ or CR⁶R⁷, bonds a and a′ are single bonds; and R⁶ and R⁷ are each independently H or C₁₋₆alkyl; or R⁶ and R⁷ together with the carbon atom on which they are substituted form a 3-6 membered optionally substituted carbocyclic ring.

In one embodiment, the compounds of formula II are selected such that R¹, R², R³ and R⁵ are follows:

i) R¹, R², R³ and R⁵ are each independently H or C₁₋₆alkyl; or

ii) R² and R³ together with the carbon atoms on which they are substituted form ring b, where ring b is a 4 membered heterocyclic; and R¹ and R⁵ are each independently H and C₁₋₆ alkyl;

R⁴ is C═CH₂ or CR⁶R⁷, and bonds a and a′ are single bonds;

R⁶ and R⁷ together with the carbon atom on which they are substituted form a 3 membered carbocyclic ring;

R⁹ is C(═O)R¹²;

R¹² is H or C₁₋₆alkyl; and

X is straight C₁₋₂alkylene.

In one embodiment, the compounds are of formula IV

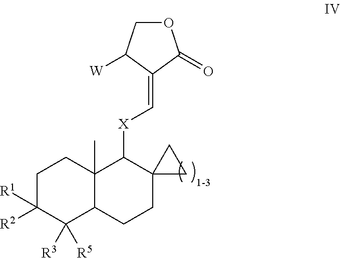

or a single stereoisomer, a mixture of stereoisomers, a racemic mixture of stereoisomers, a solvate, a hydrate, or a pharmaceutically acceptable salt thereof, wherein:

R¹, R², R³ and R⁵ are as follows:
i) R¹ and R² are each independently H, C₁₋₆alkyl, OR¹⁰, or NR^{11a}R^{11b}; and R³ and R⁵ are each independently H or C₁₋₆alkyl; provided that R¹ and R² are not both OR¹⁰ or NR^{11a}R^{11b} at the same time; or
ii) R² and R³ together with the carbon atoms on which they are substituted form ring b, where ring b is a 4 to 6 membered optionally substituted carbocyclic, heterocyclic or heteroaryl ring; and R¹ and R⁵ are each independently H or C₁₋₆ alkyl;

R¹⁰ , R^{11a} and R^{11b} are each independently H, C₁₋₆ alkyl, or 4 to 6 membered optionally substituted carbocyclic or heterocyclyc ring;

W is OH or NHR⁹;

R⁹ is C(═O)R¹² or SO₂R^{12a};

R¹² is H, C₁₋₆alkyl or OR^{12a};

R^{12a} is C₁₋₆alkyl; and

X is straight or branched C₁₋₆alkylene, optionally with one or two oxygen atoms in the chain;

where the substituents on the carbocyclic and heterocyclic rings and on the alkyl groups for R¹, R², R³, R⁵, R⁹, R¹⁰, R¹¹, R^{11a}, R¹² and R^{11a}, when present are one to three

groups Q¹, where Q¹ is C₁₋₆alkyl, hydroxy, amino, halo, C₁₋₆alkoxy, hydroxy C₁₋₆alkyl, haloC₁₋₆alkyl, aminoC₁₋₆alkyl, C₁₋₆alkoxy C₁₋₆alkyl, and C₃₋₆cycloalkyl.

In one embodiment, the compounds are of formula V

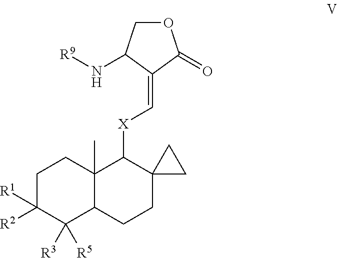

or a single stereoisomer, a mixture of stereoisomers, a racemic mixture of stereoisomers, a solvate, a hydrate, or a pharmaceutically acceptable salt thereof, wherein:

R¹, R², R³ and R⁵ are as follows:

i) R¹, R², R³ and R⁵ are each independently H or C₁₋₆alkyl; or

ii) R² and R³ together with the carbon atoms on which they are substituted form ring b, where ring b is a 4 membered heterocyclic; and R¹ and R⁵ are each independently H or C₁₋₆ alkyl;

R⁹ is C(═O)R¹² or SO₂R^{12a};

R¹² is H or C₁₋₆alkyl;

R^{12a} is C₁₋₆alkyl; and

X is straight C₁₋₂alkylene.

In one embodiment, the compounds are of formula VI

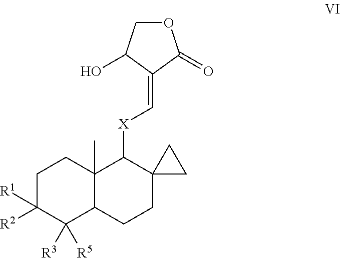

or a single stereoisomer, a mixture of stereoisomers, a racemic mixture of stereoisomers, a solvate, a hydrate, or a pharmaceutically acceptable salt thereof, wherein:

R¹, R², R³ and R⁵ are as follows:

i) R¹, R², R³ and R⁵ are each independently H or C₁₋₆alkyl; or

ii) R² and R³ together with the carbon atoms on which they are substituted form ring b, where ring b is a 4 membered heterocyclic; and R¹ and R⁵ are each independently H or C₁₋₆ alkyl; and

X is straight C₁₋₂alkylene.

In one embodiment, the compounds are of formula VII

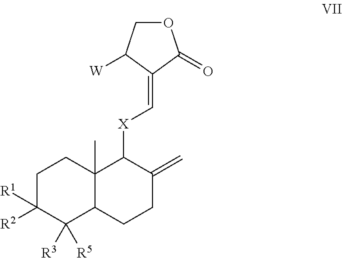

or a single stereoisomer, a mixture of stereoisomers, a racemic mixture of stereoisomers, a solvate, a hydrate, or a pharmaceutically acceptable salt thereof, wherein:

R¹, R², R³ and R⁵ are selected as follows:

i) R¹ and R² are each independently H, C₁₋₆alkyl, OR¹⁰, or NR^{11a}R^{11b}; and R³ and R⁵ are each independently H and C₁₋₆alkyl; provided that R¹ and R² are not both OR¹⁰ or NR^{11a}R^{11b} at the same time; or

ii) R² and R³ together with the carbon atoms on which they are substituted form ring b, where ring b is a 4 to 6 membered optionally substituted carbocyclic, heterocyclic or heteroaryl ring; and R¹ and R⁵ are each independently H and C₁₋₆ alkyl;

R¹⁰, R^{11a} and R^{11b} are each independently H, C₁₋₆ alkyl, 4 to 6 membered

optionally substituted carbocyclic or heterocyclyc ring;

W is OH or NHR⁹;

R⁹ is C(═O)R¹² or SO₂R^{12a};

R¹² is H, C₁₋₆alkyl or OR^{12a};

R^{12a} is C₁₋₆a1ky1; and

X is straight or branched C₁₋₆alkylene, optionally with one or two oxygen atoms in the chain;

where the substituents on the carbocyclic and heterocyclic rings and on the alkyl groups for R¹, R², R³, R⁵, R⁹, R¹⁰, R¹¹, R^{11a}, R¹² and R^{12a}, when present are one to three groups Q¹, where Q¹ is C₁₋₆alkyl, hydroxy, amino, halo, C₁₋₆alkoxy, hydroxy C₁₋₆alkyl, haloC₁₋₆alkyl, aminoC₁₋₆alkyl, C₁₋₆a1koxy C₁₋₆alkyl, or C₃₋₆cycloalkyl, where the compound is selected such that

i) when W is OH, one of R¹ or R² is OH and the other is H, and one of R³ and R⁵ is hydroxymethyl, then the other of R³ or R⁵ is H;

ii) when W is OH, one of R³ or R⁵ is CH₃ and the other is hydrogen, and one of R¹ and R² is OH, then the other of R¹ or R² is alkyl;

iii) when W is OH, at least one of R¹ or R² is OH, and one of R³ and R⁵ is aminoalkyl, then the other of R³ or R⁵ is H;

iv) when W is OH, and R¹ and R² are both H, then at least one of R³ and R⁵ is other than methyl; and

v) when W is OH, R² and R³ together with the carbon atoms on which they are substituted form a 4 membered heterocyclic ring having one oxygen atom; then R¹ is not H.

In one embodiment, the compounds are of formula VIII

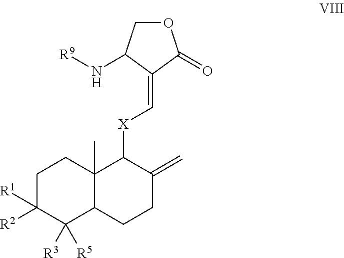

or a single stereoisomer, a mixture of stereoisomers, a racemic mixture of stereoisomers, a solvate, a hydrate, or a pharmaceutically acceptable salt thereof, wherein:

R¹, R², R³ and R⁵ are as follows:

i) R¹, R², R³ and R⁵ are each independently H or C₁₋₆alkyl; or

ii) R² and R³ together with the carbon atoms on which they are substituted form ring b, where ring b is a 4 membered heterocyclic; and R¹ and R⁵ are each independently H and C₁₋₆ alkyl;

R⁹ is C(═O)R¹² or SO₂R^{12a};

R¹² is H, C₁₋₆alkyl or OR^{12a};

R^{12a} is C₁₋₆alkyl; and

X is straight C₁₋₂alkylene.

In one embodiment, the compounds are of formula IX

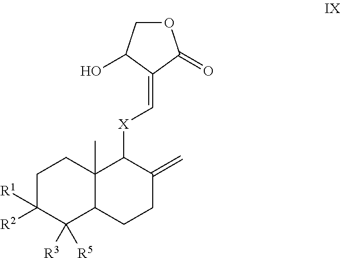

or a single stereoisomer, a mixture of stereoisomers, a racemic mixture of stereoisomers, a solvate, a hydrate, or a pharmaceutically acceptable salt thereof, wherein:

R¹, R², R³ and R⁵ are as follows:

i) R¹ and R² are each independently H, C₁₋₆alkyl, OR¹⁰, or NR^{11a}R^{11b}; and R³ and R⁵ are each independently H and C₁₋₆alkyl; provided that R¹ and R² are not both OR¹⁰ or NR^{11a}R^{11b} at the same time; or

ii) R² and R³ together with the carbon atoms on which they are substituted form ring b, where ring b is a 4 to 6 membered optionally substituted carbocyclic, heterocyclic or heteroaryl ring; and R¹ and R⁵ are each independently H and C₁₋₆ alkyl;

R¹⁰, R^{11a} and R^{11b} are each independently H or C₁₋₆ alkyl; and

X is straight C₁₋₂alkylene, where the compound is selected such that

i) when one of R¹ or R² is OH and the other is H, and one of R³ and R⁵ is hydroxymethyl, then the other of R³ or R⁵ is H;

ii) when one of R³ or R⁵ is CH₃ and the other is hydrogen, and one of R¹ and R² is OH, then the other of R¹ or R² is alkyl;

iii) when at least one of R¹ or R² is OH, and one of R³ and R⁵ is aminoalkyl, then the other of R³ or R⁵ is H;

iv) when R¹ and R² are both H, then at least one of R³ and R⁵ is other than methyl; and

v) when R² and R³ together with the carbon atoms on which they are substituted form a 4 membered heterocyclic ring having one oxygen atom; then R¹ is not H.

In one embodiment, the compounds are of formula X

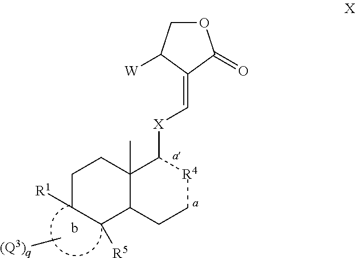

or a single stereoisomer, a mixture of stereoisomers, a racemic mixture of stereoisomers, a solvate, a hydrate, or a pharmaceutically acceptable salt thereof, wherein:

R¹ is H or C₁₋₆alkyl;

R⁵ is H or C₁₋₆ alkyl;

ring b is a carbocyclic or heterocyclic 4-6 membered ring;

Q³ is oxo;

q is 0-1;

R⁴, bond a and bond a′ are as follows:

i) R⁴ is CR⁶, bond a is a double bond, and bond a′ is a single bond; or

ii) R⁴ is CR⁶, bond a is a single bond, and bond a′ is a double bond; or

iii) R⁴ is C═CH₂ or CR⁶R⁷, and bonds a and a′ are single bonds;

R⁶ and R⁷ are each independently H or C₁₋₆alkyl; or R⁶ and R⁷ together with the carbon atom on which they are substituted form a 3-6 membered optionally substituted carbocyclic ring;

W is OH or NHR⁹;

R⁹ is C(═O)R¹² or SO₂R^{12a};

R¹² is H, C₁₋₆alkyl or OR^{12a};

R^{12a} is C₁₋₆alkyl; and

X is straight or branched C₁₋₆alkylene, optionally with one or two oxygen atoms in the chain;

where the substituents on the carbocyclic and heterocyclic rings and on the alkyl groups for R¹, R⁵, R⁹, R¹⁰, R¹¹, R^{11a}, R¹² and R^{12a}, when present are one to three groups Q¹, where Q¹ is C₁₋₆alkyl, hydroxy, amino, halo, C₁₋₆alkoxy, hydroxy C₁₋₆alkyl, haloC₁₋₆alkyl, aminoC₁₋₆alkyl, C₁₋₆a1koxy C₁₋₆alkyl, and C₃₋₆cycloalkyl, where

i) when W is OH, R⁴ is C—CH₃, and bond ring b is a five membered ring containing two heteroatoms, then at least one heteroatom in ring b is other than nitrogen; and

ii) when W is OH and ring b is a six membered heterocyclic ring containing two oxygen atoms, then ring b contains at least one additional heteroatom.

In one embodiment, the compounds are of formula XI

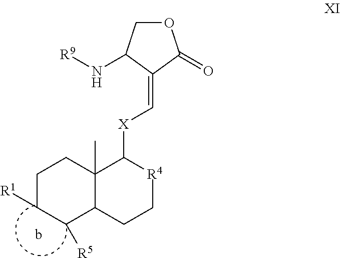

or a single stereoisomer, a mixture of stereoisomers, a racemic mixture of stereoisomers, a solvate, a hydrate, or a pharmaceutically acceptable salt thereof, wherein:

R¹ is H or C₁₋₆alkyl;

R⁵ is H or C₁₋₆ alkyl;

ring b is a carbocyclic or heterocyclic 4-6 membered ring;

R⁴ is C═CH₂ or CR⁶R⁷,

R⁶ and R⁷ together with the carbon atom on which they are substituted form a 3-6 membered carbocyclic ring;

R⁹ is C(═O)R¹² or SO₂R^{12a};

R¹² is H, C₁₋₆alkyl or OR^{12a};

R^{12a} is C₁₋₆alkyl; and

X is straight or branched C₁₋₆alkylene.

In one embodiment, the compounds are of formula XII

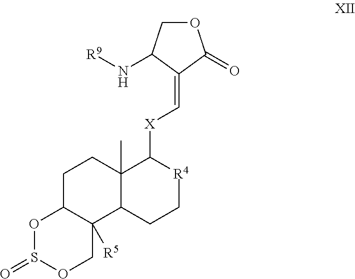

or a single stereoisomer, a mixture of stereoisomers, a racemic mixture of stereoisomers, a solvate, a hydrate, or a pharmaceutically acceptable salt thereof, wherein:

R⁵ is H or C₁₋₆ alkyl;

R⁴ is C═CH₂ or CR⁶R⁷,

R⁶ and R⁷ together with the carbon atom on which they are substituted form a 3-6 membered carbocyclic ring;

R⁹ is C(═O)R¹² or SO₂R^{12a};

R¹² is H, C₁₋₆alkyl or OR^{12a};

R^{12a} is C₁₋₆alkyl; and

X is straight or branched C₁₋₆alkylene.

In one embodiment, the compounds are of formula XIII

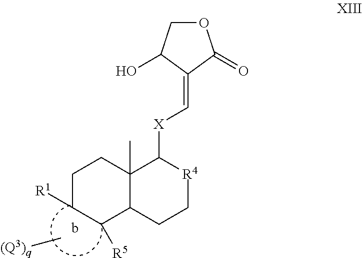

or a single stereoisomer, a mixture of stereoisomers, a racemic mixture of stereoisomers, a solvate, a hydrate, or a pharmaceutically acceptable salt thereof, wherein:

R¹ is H or C₁₋₆alkyl;

R⁵ is H or C₁₋₆ alkyl;

ring b is a carbocyclic or heterocyclic 4-6 membered ring;

Q³ is oxo;

q is 0-1;

R⁴ is C═CH₂ or CR⁶R⁷;

R⁶ and R⁷ together with the carbon atom on which they are substituted form a 3-6 membered optionally substituted carbocyclic ring; and

X is straight or branched C₁₋₆alkylene, where i) when R⁴ is C—CH₃, and bond ring b is a five membered ring containing two heteroatoms, then at least one heteroatom in ring b is other than nitrogen; and

ii) when and ring b is a six membered heterocyclic ring containing two oxygen atoms, then ring b contains at least one additional heteroatom.

In one embodiment, the compounds provided herein are of formula XIV:

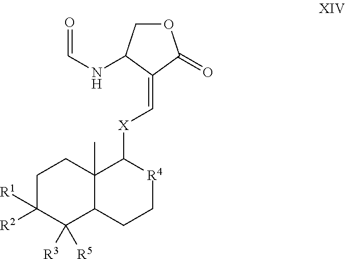

or a single stereoisomer, a mixture of stereoisomers, a racemic mixture of stereoisomers, a solvate, a hydrate, or a pharmaceutically acceptable salt thereof, wherein:

R¹, R², R³ and R⁵ are as follows:

i) R¹ and R² are each independently H, C₁₋₆alkyl, OR¹⁰, or NR^{11a}R^{11b}; and R³ and R⁵ are each independently H, hydroxyC₁₋₆alkyl or C₁₋₆alkyl; provided that R¹ and R² are not both OR¹⁰ or NR^{11a}R^{11b} at the same time; or

ii) R² and R³ together with the carbon atoms on which they are substituted form ring b, where ring b is a 4 to 6 membered carbocyclic, heterocyclic or heteroaryl ring; ring b is optionally substituted with an oxo group, and R¹ and R⁵ are each independently H and C₁₋₆ alkyl;

R¹⁰, R^{11a} and R^{11b} are each independently H, C₁₋₆ alkyl, or 4 to 6 membered carbocyclic or heterocyclyc ring;

R⁴ is C═CH₂ or CR⁶R⁷;

R⁶ and R⁷ together with the carbon atom on which they are substituted form a 3-6 membered carbocyclic ring; and

X is straight or branched C₁₋₆alkylene.

In one embodiment, the compounds are of formula XV

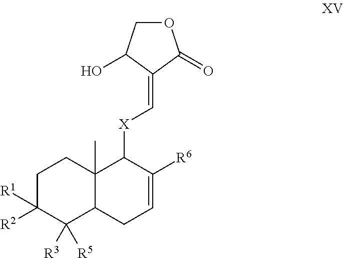

or a single stereoisomer, a mixture of stereoisomers, a racemic mixture of stereoisomers, a solvate, a hydrate, or a pharmaceutically acceptable salt thereof, wherein:

R¹, R², R³ and R⁵ are selected as follows:

i) R¹ and R² are each independently H, C₁₋₆alkyl, OR¹⁰, or NR^{11a}R^{11b}; and R³ and R⁵ are each independently H or C₁₋₆alkyl; provided that R¹ and R² are not both OR¹° or NR^{11a}R^{11b} at the same time; or

ii) R² and R³ together with the carbon atoms on which they are substituted form ring b, where ring b is a 4 to 6 membered optionally substituted carbocyclic, heterocyclic or heteroaryl ring; and R¹ and R⁵ are each independently H or C₁₋₆ alkyl;

R¹⁰, R^{11a} and R^{11b} are each independently H, C₁₋₆ alkyl, 4 to 6 membered optionally substituted carbocyclic or heterocyclyc ring;

R⁶ is H or C₁₋₆alkyl; and

X is straight or branched C₁₋₆alkylene, optionally with one or two oxygen atoms in the chain;

where the substituents on the carbocyclic and heterocyclic rings and on the alkyl groups for R¹, R², R³, R⁵, R¹⁰, R¹¹, and R^{11a}, when present are one to three groups Q¹, where Q¹ is C₁₋₆alkyl, hydroxy, amino, halo, C₁₋₆alkoxy, hydroxy C₁₋₆alkyl, haloC₁₋₆alkyl, aminoC₁₋₆alkyl, C₁₋₆alkoxy C₁₋₆alkyl, or C₃₋₆cycloalkyl.

In one embodiment, the compounds are of any of formula I-IX and XIV-XV, wherein:

R¹ is hydrogen;

R² is OR¹⁰;

R³ is C₁₋₆alkyl;

R⁵ is hydrogen;

R¹⁰ is hydrogen or C₁₋₆alkyl; and

X is straight or branched C₁₋₂alkylene.

In one embodiment, the compounds are of any of formula I-X and XII-XV, wherein:

R¹ and R⁵ are is hydrogen or C₁₋₆alkyl;

R² and R³ together with the carbon atoms on which they are substituted form ring b, where ring b is a 4 membered heterocyclic ring; and

X is straight or branched C₁₋₂alkylene.

In one embodiment, the compounds are of formula XXI

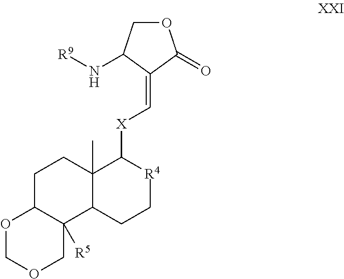

or a single stereoisomer, a mixture of stereoisomers, a racemic mixture of stereoisomers, a solvate, a hydrate, or a pharmaceutically acceptable salt thereof, wherein:

R⁵ is H or C₁₋₆ alkyl;

R⁴ is C═CH₂ or CR⁶R⁷,

R⁶ and R⁷ together with the carbon atom on which they are substituted form a 3-6 membered carbocyclic ring;

R⁹ is C(═O)R¹² or SO₂R^{12a};

R¹² is H, C₁₋₆alkyl, C₁₋₆alkoxy C₁₋₆alkyl or OR^{12a};

R^{12a} is C₁₋₆alkyl; and

X is straight or branched C₁₋₆alkylene.

In one embodiment, the compound is selected from the following:

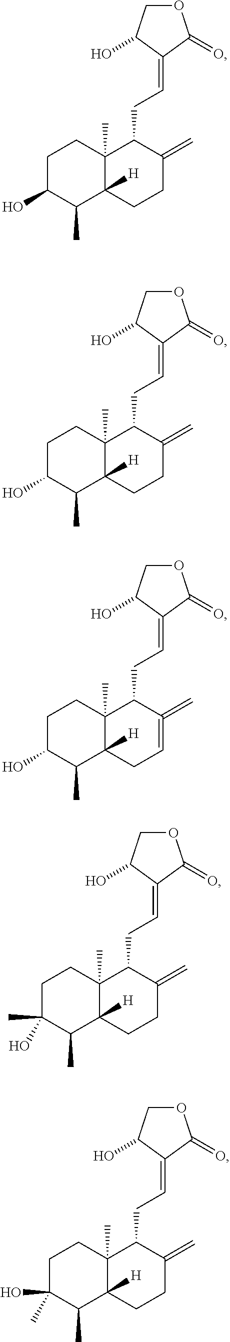

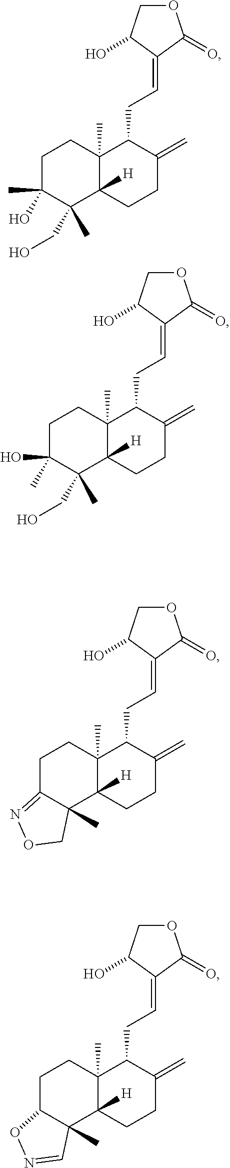

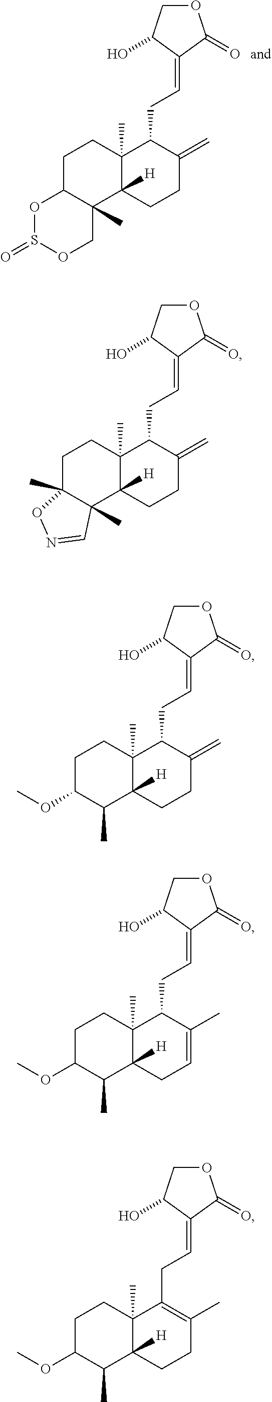

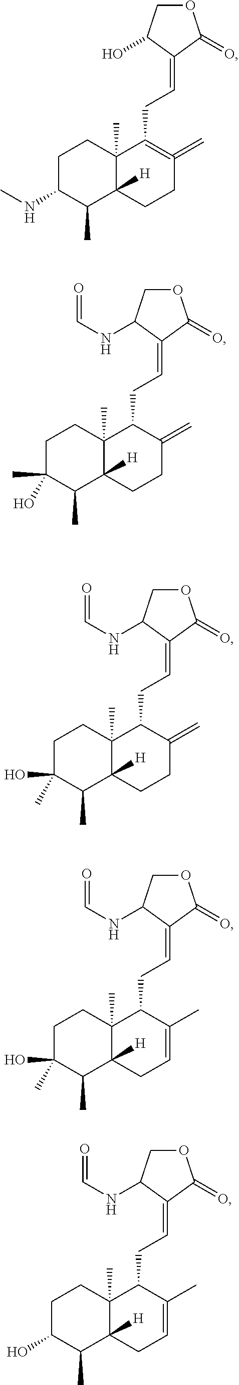

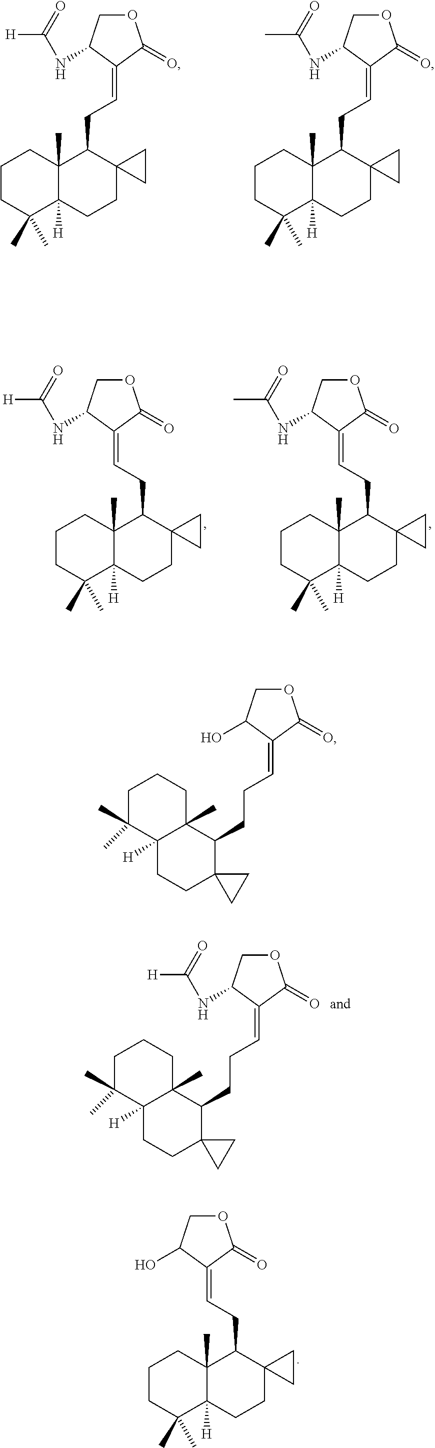

In one embodiment, the compound is selected from the following:

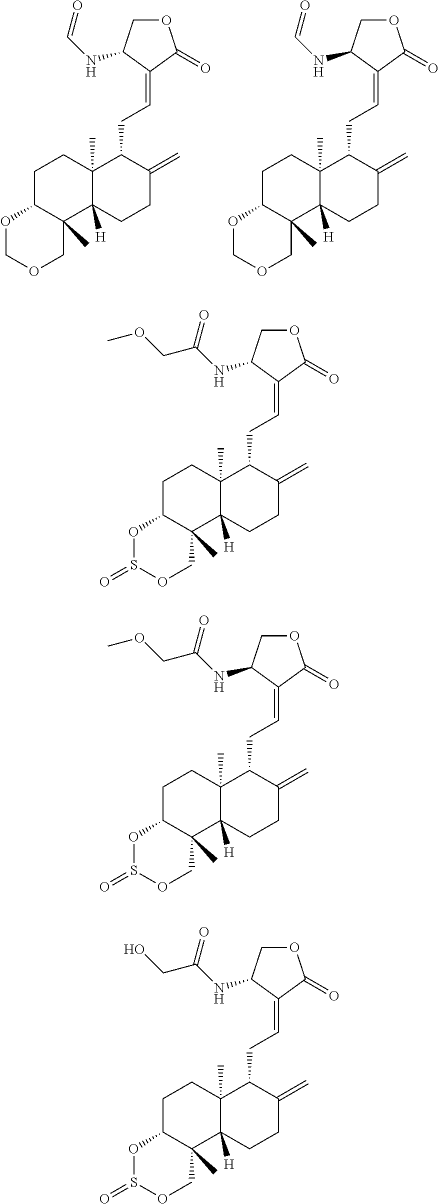

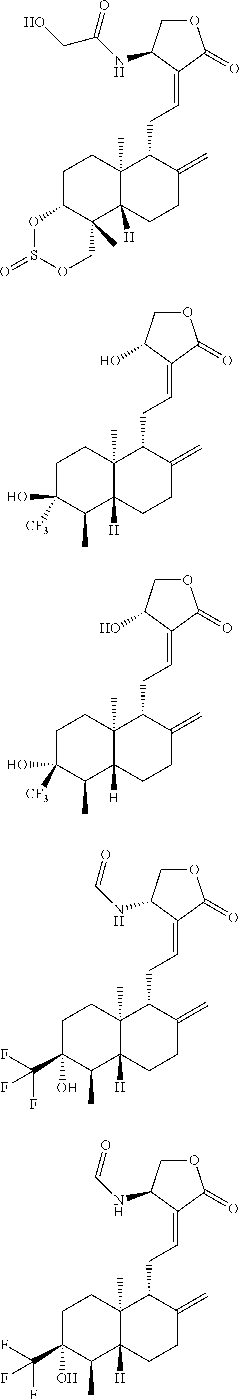

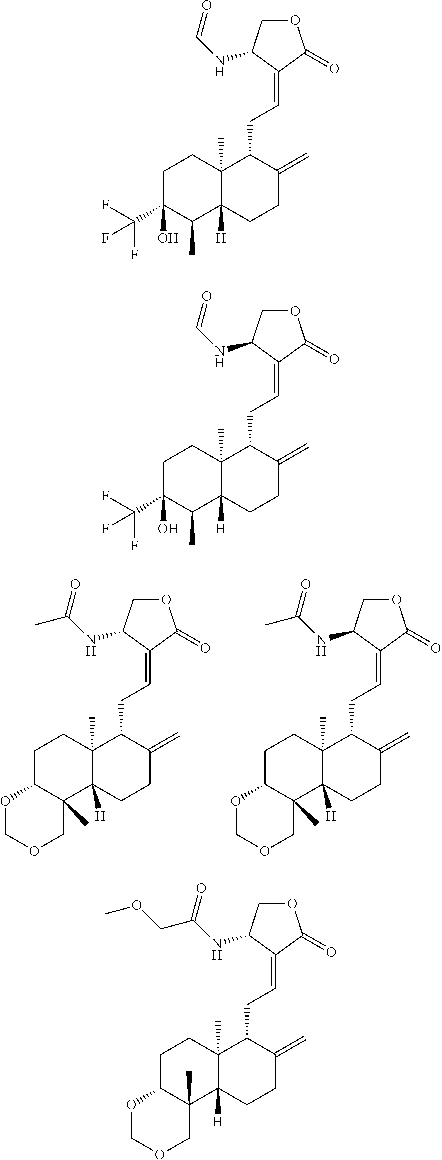

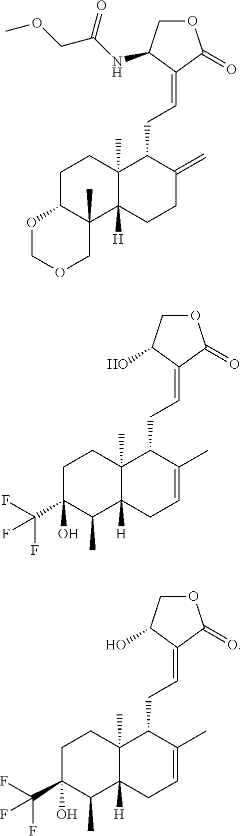

Preparation of Compounds

The compounds provided herein can be prepared by methods known to one of skill in the art and following procedures similar to those described in the Examples section herein and routine modifications thereof.

Certain exemplary reaction schemes for the preparation of compounds are illustrated below.

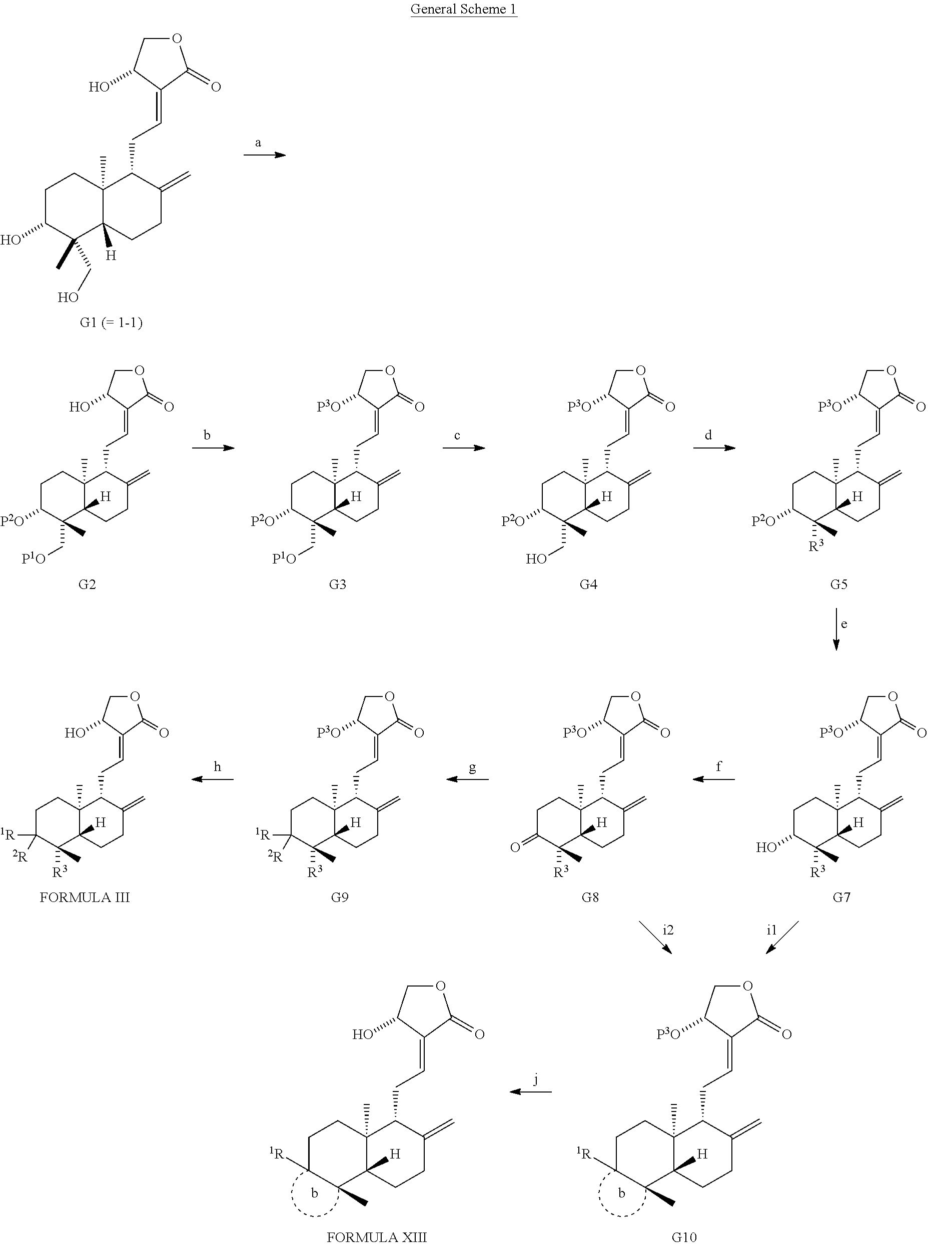

Some of the compounds in this invention can be prepared following General Scheme 1 starting with the commercially available Angrographolide (G1 or 1-1). The three hydroxyl groups of G1 can be differentially protected to provide intermediates G2 and G3. Specific examples of the protecting groups can be found in the experimental section including the silyl protecting groups and benzyl ketal group. Selective de-protection of P1 group and subsequent transformation of the primary alcohol in G4 would install suitable R3 group to give G5. Removal of P2 gives G7 and further oxidation using common oxidant such as Dess-Martin Periodinate would lead to G8.

The ketone functional group in G8 can be further transformed to afford desired R1 and R2 substituent groups via organolithium addition, or reduction and alkylation, and or reductive amination amongst others. Final removal of P3 group would generate the target compounds of Formula III.

From G7 and G8 intermediates, common functional group manipulations including cyliczations may generate ring b to provide intermediate G10. Final removal of protection group P3 can generate compounds of Formula XIII.

In many expamples, the protection schemes of the three hydroxyl groups can be varied or simplified depending on the specific substrates. Illustrative examples can be found in the preparation of Examples 1 to 27.

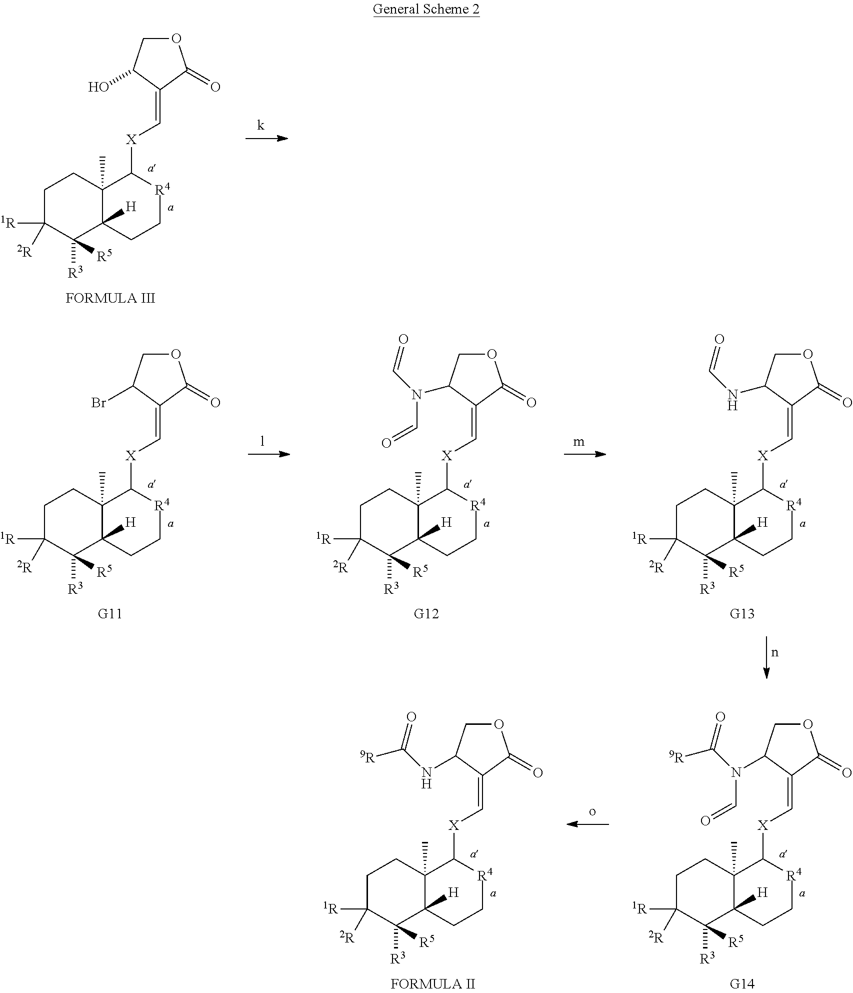

Compounds of Formula II can be prepared from compounds of Formula III via the synthetic approach described in the General Scheme 2. The hydroxyl group in Formula III can be converted to the bromide under the conditions of carbon tetrabromide and triphenylphosphine. Displacement of the bromide with sodium diformamide followed by mild hydrolysis of one of the formyl group will provide key intermediates G13 (which itself is part of Formula II). Under basic condition such as LiHMDS, G13 can be converted to G14 using a variety of electrophiles R⁹X in step n. Removal of the final formyl group in G14 affords compounds of Formula II.

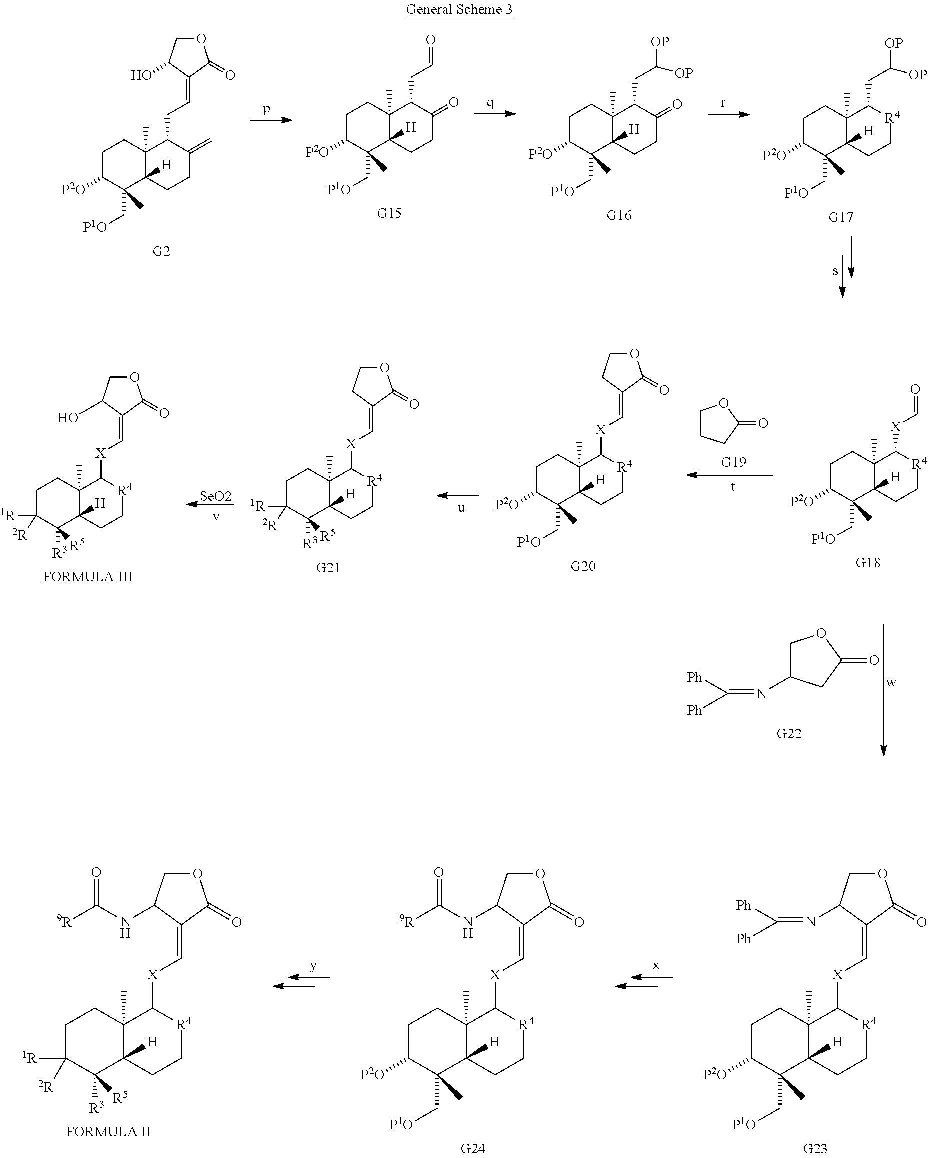

General Scheme 3 outlines an alternative transformation pathway to some compounds of Formula I, specifically Formula III and Formula II. Under the conditions of ozonolysis, intermediate G2 or similar compounds can be converted to G15. Selective protection of the aldehyde would provide G16, which subsequently undergoes transformations to G17. Removal of the aldehyde protecting group and further functional group manipulation would produce G18 with the desirable X linker. Aldol codensation of G18 with G19 provides G20. Following the methods used in general schemes 1 and 2, the OP2 and OP1 groups can be converted in a few steps to suitable R¹, R², and R³ groups in G21. Upon oxidative treatment of G21 with selenium dioxide generates the target compounds of Formula III.

Aldol condensation of G18 with G22 will generate the imine intermediate G23. Cleavage of the benzoimine protecting group to reveal a free amino functional group. This amino group can be further elaborated for example via acylation and alkylation to afford intermediate G24. Similar chemistry in general scheme 1 and 2 can be applied to convert G24 to compounds of formula II.

Formulation of Pharmaceutical Compositions

The pharmaceutical compositions provided herein contain therapeutically effective amounts of one or more of compounds provided herein and a pharmaceutically acceptable carrier, diluent or excipient.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the compounds in the compositions are effective for delivery of an amount, upon administration, that treats, prevents, or ameliorates one or more of the symptoms and/or progression of a disease associated with Nrf2/ NF-κB pathways.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the 7flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of a disease associated with Nrf2/ NF-κB pathways.

In certain embodiments, a therapeutically effective dosage should produce a serum concentration of active ingredient of from about 0.1 ng/ml to about 50-100 μg/ml. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing . The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to orally, parenterally, rectally, topically and locally. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S--S bond formation through thio-disulfide interchange, stabilization may be achieved by modifying sulfhydryl residues, lyophilizing from acidic solutions, controlling moisture content, using appropriate additives, and developing specific polymer matrix compositions

Dosage forms or compositions containing active ingredient in the range of 0.005% to 100% with the balance made up from non toxic carrier may be prepared. For oral administration, a pharmaceutically acceptable non toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1 85% or about 75-95%.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

Lactose-free compositions provided herein can contain excipients that are well known in the art and are listed, for example, in the U.S. Pharmocopia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable salt thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non effervescent granules and effervescent preparations reconstituted from effervescent granules. Aqueous solutions include, for example, elixirs and syrups. Emulsions are either oil in-water or water in oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non aqueous liquids, emulsifying agents and preservatives. Suspensions use pharmaceutically acceptable suspending agents and preservatives. Pharmaceutically acceptable substances used in non effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461,6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

Targeted Formulations

The compounds provided herein, or pharmaceutically acceptable salts thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

Articles of Manufacture

The compounds or pharmaceutically acceptable salts can be packaged as articles of manufacture containing packaging material, a compound or pharmaceutically acceptable salt thereof provided herein, which is used for treatment, prevention or amelioration of one or more symptoms or progression of disease associated with Nrf2/NF-κB pathways, and a label that indicates that the compound or pharmaceutically acceptable salt thereof is used for treatment, prevention or amelioration of one or more symptoms or progression of such disease.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

Methods of Use of the Compounds and Compositions

In one aspect, provided herein are methods of treating, prophylaxis, or amelioration of a disease by administering to a subject in need thereof one or more compounds of formula I. Examples of such diseases include neurodegenerative diseases including multiple sclerosis (MS) (e.g., relapsing-remitting MS, secondary progressive MS, primary progressive MS, progressive relapsing MS), amyotrophic lateral sclerosis (ALS), Alzheimer's disease (AD), Parkinson's disease (PD) and Huntington's disease (HD).

Other examples of neurodegenerative diseases include acute haemorrhagic leucoencephalomyelitis, Hurst's disease, encephalomyelitis (e.g., acute disseminated encephalomyelitis), optic neuritis, spinal cord lesions, acute necrotizing myelitis, transverse myelitis, chronic progressive myelopathy, progressive multifocal leukoencephalopathy (PML), radiation myelopathy, HTLV-1 associated myelopathy, monophasic isolated demyelination, central pontine myelinolysis, leucodystrophy (e.g., adrenoleucodystrophy, metachromatic leucodystrophy, Krabbe's disease, Canavan's disease, Alexander's disease, Pelizaeus-Merbacher disease, vanishing white matter disease, oculodentodigital syndrome), inflammatory demyelinising polyneuropathy (e.g., chronic inflammatory demyelinating polyneuritis (CIDP), and acute inflammatory demyelinating polyneuropathy (AIDP)).

Additional examples of diseases suitable for the methods provided herein include Guillain-Barre syndrome (GBS), polyneuritis, myasthenia gravis (MG), Eaton Lambert Syndrome (ELS), and encephalomyelitis. These disorders may be co-presented with, and possibly aggravated by diabetes, e.g., insulin-dependent diabetes mellitus (IDDM; type I diabetes), or other diseases.

Other examples of diseases suitable for the methods provided herein are diseases associated with fibrosis including Idiopathic Pulmonary Fibrosis (IPF), Scleroderma lung disease, Acute Lung Injury (ALI)/Acute respiratory Distress (ARDS), Chronic Asthma, Radiation-Induced Fibrosis Sarcoidosis, Pulmonary Hypertension, Bronchopulmonary Dysplasia (BPD), Lung Transplant Rejection, Pulmonary GVHD Complications, Interstitial pneumonia Syndrome (IPS) in transplant recipients, COPD, Silicosis, Asbestosis, Sarcoidosis (lung), Primary sclerosing cholangitis (PSC), Alcohol-induced hepatic fibrosis, Autoimmune hepatitis, Chronic viral hepatitis (HepB,C), Primary biliary cirrhosis (PBC), Non-alcohol Steatohepatitis (NASH), Liver transplant rejection, Hepatic complications of GVHD, Veno-occlusive disease in transplant recipients, Focal Segmental Glomerular Sclerosis (FSGS), Diabetic nephropathy, IgA nephropathy, Scleroderma, Renal complications of GVHD (AKI delayed graft function), Acute renal failure post CABG (AKI post CABG), Lupus nephritis, Hypertension-induced Renal Fibrosis, HIV-associated nephropathy, Peritoneal dialysis-induced peritoneal fibrosis, Retroperitoneal fibrosis, Idiopathic Glomerulosclerosis, Kidney transplant rejection, Alport syndrome, Restenosis, Subarachnoid hemorrhage (SAH), Heart transplant rejection, Stroke, Cosmetic surgery, Chronic wounds, Burns, Surgical adhesions, Keloids, Donor graft re-epithelialization, Myelofibrosis, Corneal transplant, LASIX, Trabeculectomy, Systemic sclerosis, Radiation induced fibrosis, Peripatellar Fibrosis, and Dupuytren's Contractures. In one aspect, the fibrosis disease is sclederma.

Other diseases for which compounds of formula I may be therapeutically effective include inflammatory bowel disease, Crohn's disease, lupus (e.g., Neuropsychiatric lupus), systemic Lupus erythematodes (SLE), asthma, Leber's disease, Devic's disease (NMO), Friedrich's Ataxia, mitochondrial Central Nervous System diseases, scleroderma, uveitis, anti-phospholipid antibody syndrome, polyarthritis (e.g., rheumatoid arthritis), polyarticular juvenile idiopathic arthritis, sickle cell disease, ankylosing spondylitis, myositis, atherosclerosis, diabetic peripheral neuropathy, head injury, stroke, HIV-dementia, myocardial infarction, angina pectoris, cardiac insufficiency, psoriasis, psoriatic arthritis, Sjogren's syndrome, diabetes (e.g., type 1 diabetes, diabetes mellitus type II, juvenile-onset diabetes), blistering skin diseases, sarcoidosis, osteoarthritis, ulcerative colitis, vasculitis, lung fibrosis, idiopathic pulmonary fibrosis (IPF), liver fibrosis, kidney fibrosis, acute kidney injury, chronic kidney disease—diabetic nephrophathy, graft-versus-host reactions, Hashimoto's thyroiditis, Grave's disease, pernicious anaemia, hepatitis (e.g., chronic acid (=lupoid) hepatitis, acute hepatitis, toxic hepatitis, alcohol-induced hepatitis, viral hepatitis, jaundice, liver insufficiency, and cytomegaloviral hepatitis), neurodermatitis, retinopathia pigmentosa, forms of mitochondrial encephalomyopathy, osteochondritis syphilitica (Wegener's disease), cutis marmorata (livedo reticularis), Behcet disease, panarteriitis, osteoarthritis, gout, artenosclerosis, Reiter's disease, pulmonary granulomatosis, types of encephalitis, endotoxic shock (septic-toxic shock), sepsis, pneumonia, anorexia nervosa, Rennert T-lymphomatosis, mesangial nephritis, post-angioplastic restenosis, reperfusion syndrome, cytomegaloviral retinopathy, adenoviral diseases (e.g., adenoviral colds, adenoviral pharyngoconjunctival fever and adenoviral ophthalmia), AIDS, post-herpetic or post-zoster neuralgia, mononeuropathia multiplex, mucoviscidosis, Bechterew's disease, Barett oesophagus, Epstein-Barr virus (EVB) infection, cardiac remodeling, interstitial cystitis, human tumour radiosensitisation, multi-resistance of malignant cells to chemotherapeutic agents (multidrug resistance in chemotherapy), granuloma annulare and cancers (e.g., mamma carcinoma, colon carcinoma, melanoma, primary liver cell carcinoma, adenocarcinoma, kaposi's sarcoma, prostate carcinoma, leukaemia (e.g., acute myeloid leukaemia, multiple myeloma (plasmocytoma), Burkitt lymphoma and Castleman tumour)), chronic obstructive pulmonary diseases, PDGF induced thymidine uptake of bronchial smooth muscle cells, bronchial smooth muscle cell proliferation, Adrenal Leukodystrophy (ALD), Alcoholism, Alper's disease, Ataxia telangiectasia, Batten disease (also known as Spielmeyer-Vogt-Sjögren-Batten disease), Bovine spongiform encephalopathy (BSE), Cerebral palsy, Cockayne syndrome, Corticobasal degeneration, Creutzfeldt-Jakob disease, Familial Fatal Insomnia, Frontotemporal lobar degeneration, Kennedy's disease, Lewy body dementia, Neuroborreliosis, Machado-Joseph disease (Spinocerebellar ataxia type 3), Multiple System Atrophy, Narcolepsy, Niemann Pick disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spinocerebellar ataxia, Spinal muscular atrophy, Steele-Richardson-Olszewski disease, Tabes dorsalis, Toxic encephalopathy, MELAS (Mitochondrial Encephalomyopathy; Lactic Acidosis; Stroke), MERRF (Myoclonic Epilepsy; Ragged Red Fibers), PEO (Progressive External Opthalmoplegia), Leigh's Syndrome, MNGIE (Myopathy and external ophthalmoplegia; Neuropathy; Gastro-Intestinal; Encephalopathy), Kearns-Sayre Syndrome (KSS), NARP, Hereditary Spastic Paraparesis, Mitochondrial myopathy,optic neuritis, progressive multifocal leucoencephalopathy (PML), or other hereditary disorders (e.g., leukodystrophies, Charcot-Marie-Tooth disease), Pyoderma Gangrenosum, Erosive Pustular Dermatosis of the Scalp, Sweet's Syndrome, Bowel-associated Dermatosis-arthritis Syndrome, Pustular Psoriasis, Acute Generalized Exanthematous Pustulosis, Keratoderma Blenorrhagicum, Sneddon-Wilkinson Disease, Amicrobial Pustulosis of the Folds, Infantile Acropustulosis, Transient Neonatal Pustulosis, Neutrophilic Eccrine Hidradenitis, Rheumatoid Neutrophilic Dermatitis, Neutrophilic Urticaria, Still's Disease, Erythema Marginatum, Unclassified Periodic Fever Syndromes/Autoinflammatory Syndromes, Bullous Systemic Lupus Erythematosus, Neutrophilic Dermatosis of the Dorsal Hands (Pustular Vasculitis), anaphylaxis, allergic rhinitis, allergic asthma, lung cancer, severe asphyxic episodes of asthma, acute lung injury, Acute Respiratory Distress Syndrome, ischemia reperfusion injury, septicemia with multiorgan failure, inderteminate colitis, sickle cell crisis, or acute chest syndrome.

In one embodiment, the disease is lung fibrosis, IPF, kidney fibrosis, acute kidney injury, chronic kidney injury, and scleroderma.

In one embodiment, the disease is Sickle Cell Disease (SCD).

In one aspect, provideded herein are methods of treating, prophylaxis, or amelioration of a neurological disease by administering (e.g., orally) to a subject in need thereof one or more compounds of formula I. In one aspect, the neurological disease is MS (e.g., relapsing-remitting MS, secondary progressive MS, primary progressive MS, progressive relapsing MS), amyotrophic lateral sclerosis (ALS), Alzheimer's disease, Parkinson's disease or Huntington's disease. In one aspect, the neurological disease is MS (e.g., relapsing-remitting MS, secondary progressive MS, primary progressive MS, progressive relapsing MS). In one aspect, the neurological disease is relapsing-remitting MS.

Combination therapy

In certain embodiment, ptovided herein are methods for treating, prophylaxis, or amelioration of a subject having a neurodegenerative disease by combination therapy. For example, the methods include administering to a subject having or at risk of developing a neurodegenerative disease with a compound of formula I and one or more other compounds of formula I or one or more other therapeutic agents.

In one embodiment, the one or more other therapeutic agents is a disease modifying agent. In one embodiment, the one or more other therapeutic agents alleviate the side effects of the compound of formula I. For example, if a compound of formula I causes side effects such as flushing or GI disturbance (e.g., diarrhea), the one or more other therapeutic agent can be a therapeutic agent that can reduce the flushing (e.g., aspirin) or GI disturbance (e.g., loperamide).

In one embodiment, the first compound and the second compound may be administered concurrently (as separate compositions or together in a single dosage form) or consecutively over overlapping or non-overlapping intervals. In the sequential administration, the first compound and the second compound can be administered in any order. In some embodiments, the length of an overlapping interval is more than 1, 2, 4, 6, 12, 24, 48 weeks or longer.

In one embodiment, the compound of formula I and the one or more other therapeutic agents can be used to treat MS. The one or more other therapeutic agents can be, e.g., interferon beta-1a (Avonex®, Rebif®), glatiramer (Copaxone®), modafinil, azathioprine, predisolone, mycophenolate, mofetil, mitoxantrone, natalizumab (Tysabri®), sphinogosie-1 phosphate modulator e.g., fingolimod (Gilenya®), and other drugs useful for MS treatment such as teriflunomide (Aubagio®), piroxicam, and phenidone.

In one embodiment, the compound of formula I and the one or more other therapeutic agents can be used to treat ALS. The one or more other therapeutic agents is an agent or agents known or believe to be effective for ALS treatment, e.g., riluzole and dexpramipexole.

In one embodiment, the compound of formula I and the one or more other therapeutic agents can be used to treat AD. The one or more other therapeutic agents is an agent or agents known or believe to be effective for Alzheimer's disease treatment, e.g., rosiglitazone, roloxifene, vitamin E, donepezil, tacrine, rivastigmine, galantamine, and memantine.

In one embodiment, the compound of formula I and the one or more other therapeutic agents can be used to treat Parkinson's disease. The one or more other therapeutic agents is an agent or agents known or believe to be effective for Parkinson's disease treatment include, but are not limited to, dopamine precursors such levodopa, dopamine agonists such as bromocriptine, pergolide, pramipexole, and ropinirole, MAO-B inhibitors such as selegiline, anticholinergic drugs such as benztropine, trihexyphenidyl, tricyclic antidepressants such as amitriptyline, amoxapine, clomipramine, desipramine, doxepin, imipramine, maprotiline, nortriptyline, protriptyline, amantadine, and trimipramine, some antihistamines such as diphenhydramine; antiviral drugs such as amantadine.

In one embodiment, the compound of formula I and the one or more other therapeutic agents can be used to treat Huntington's disease. Useful drugs for treating, prophylaxis, or amelioration of symptoms of Huntington's disease further include, but are not limited to, selective serotonin reuptake inhibitors (SSRI) such as fluoxetine, paroxetine, sertraline, escitalopram, citalopram, fluvosamine; norepinephrine and serotoiun reuptake inhibitors (NSRI) such as venlafaxine and duloxetine.

Methods of Evaluating Nrf2 Activators

In certain embodiments, the neuroprotective effects of compounds of formula I can be investigated in the malonate striatal lesion model of excitotoxicity. Malonate is a succinate dehydrogenase inhibitor, which is a mitochondrial enzyme that plays a central role in neuronal energy metabolism. Injection of malonate into the striatal region of the brain produces a lesion that is excitotoxic in character, as it can be blocked by systemic administration of N-methyl-D-aspartate (NMDA) receptor antagonists and has little inflammatory involvement. Intrastriatal malonate injection has been used as a model for acute neurodegeneration, and the potential therapeutic effects of test compounds of formula I can be explored in this setting. See, e.g., Scannevin R. H. et al., poster P02.121, 64^{th} Annual Meeting of the American Academy of Neurology, Apr. 21-28, 2012, New Orleans, La., USA.

In certain embodiments, the mouse cuprizone/rapamycin model of demyelination and neurodegeneration can be used to evaluate the neuroprotective effects of compounds of formula I. Specifically, cuprizone is a neurotoxicant that when administered chronically to mice results in demyelination in the central nervous system, and has been used as a model to investigate modulation of remyelination. Administering rapamycin in addition to cuprizone results in more robust and consistent demyelination, presumably due to the anti-proliferative effect of stimulating the mammalian target of rapamycin (mTOR) receptor and pathway. The cuprizone plus rapamycin injury paradigm models prevalent pathologies (e.g., axonal transection, formation of ovoids and neuronal degeneration) associated with the human disease, and observation using this model provides unique insights into the mechanism of action of test compounds.

In certain embodiment, compounds of formula I can be assayed in MS animal model, such as Experimental Autoimmune Encephalomyelitis (EAE) (Tuohy et al., J. Immunol., 1988, 141:1126-1130, Sobel et al. J. Immunol., 1984, 132:2393-2401, and Traugott, Cell Immunol., 1989 119:114-129). Chronic relapsing EAE provides a well established experimental model for testing agents that would be useful for the treatment of MS. The mouse EAE is an induced autoimmune demyelinating disease with many similarities to human MS in its clinical manifestations. Other animal models that can be used include Thieler's murine encephalomyelitis virus (TMEV)-induced demyelinating disease, murine hepatitis virus (MHV), Semliki Forest Virus, and Sindbis virus as described in, e.g., Ercoli et al., J. Immunol., 2006, 175:3293-3298.

In certain embodiment, compounds of formula I can be assayed in an ALS animal model, such as the mouse model with ALS-linked SOD1 G93A mutation. In certain embodiment, compounds of formula I can be assayed in an hSOD1 G93A animal model. See Vargas M. R., et al., J. Neurosci., 2008, 28(50):13574-13581.

In certain embodiment, compounds of formula I can be assayed in Alzheimer's disease animal models such as spontaneous models in various species, including senescence-accelerated mice, chemical and lesion-induced rodent models, and genetically modified models developed in Drosophila melanogaster, Caenorhabditis elegans, Danio rerio and rodents. For review, see, e.g., Van Dam et al., Br. J. Pharmacol. 2011, 164(4):1285-1300 and Gotz et al., Nat. Rev. Neurosci.2008, 9:532-544.

In certain embodiment, compounds of formula I can be assayed in Parkinson's disease animal models, including toxin-based (those produced by 6-hydroxydopamine (6-OHDA), 1-methyl-1,2,3,6-tetrahydropiridine (MPTP) rotenone, and paraquat) or genetic models such as those utilizing the in vivo expression of Parkinson's disease-related mutations (e.g., those related to alpha-synuclein, PINK1, Parkin and LRRK2). For review, see, e.g., Blesa et al., J Biomed. Biotech.2012, Article ID 845618, pages 1-10.

In certain embodiment, compounds of formula I can be assayed in HD animal model, such as, toxin-induced models or genetic models. Toxin-induced models (e.g., those based on 3-nitropropionic acid and quinolinic acid) are used to study mitochondrial impairment and excitotoxicity-induced cell death, which are both mechanisms of degeneration seen in the HD brain. The discovery of the HD genetic mutation that led to HD in 1993 has led HD animal models that are genetic-based. These models include transgenic and knock-out mice, as well as a model that uses a viral vector to encode the gene mutation in certain areas of the brain. For review, see, e.g., Ramaswamy et al., ILAR J2007; 48(9):356-373.

The compounds provided herein may be optionally tested in at least one additional animal model (see, generally, Immunologic Defects in Laboratory Animals, eds. Gershwin et al., Plenum Press, 1981), for example, such as the following: the SWR X NZB (SNF1) mouse model (Uner et al., J. Autoimmune Disease, 1998, 11(3):233-240), the KRN transgenic mouse (K/BxN) model (Ji et al., Immunol. Rev., 1999, 69:139); NZB X NZW (B/W) mice, a model for SLE (Riemekasten et al., Arthritis Rheum., 2001,) 44(10):2435-2445); the NOD mouse model of diabetes (Baxter et al., Autoimmunity, 1991, 9(1):61-67), etc.). Exemplary assays and results are described in the Examples section below.

It will be appreciated that every suitable combination of the compounds provided herein with one or more of the aforementioned compounds and optionally one or more further pharmacologically active substances is contemplated herein.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

The compounds provided herein are prepared by the synthetic procedures known in the art and described herein. Synthetic procedures for exemplary compounds are described in Examples 1-38.

### Examples 1 and 2

### Synthesis of (S,E)-4-hydroxy-3-(2-((1R,4aR,5R,6S,8aR)-6-hydroxy-5,8a-dimethyl-2-methylenedecahydronaphthalen-1-yl)ethylidene)dihydrofuran-2(3H)-one (Ex.1)

### and (S,E)-4-hydroxy-3-(2-((1R,4aR,5R,6R,8aR)-6-hydroxy-5,8a-dimethyl-2-methylenedecahydronaphthalen-1-yl)ethylidene)dihydrofuran-2(3H)-one (Ex.2)

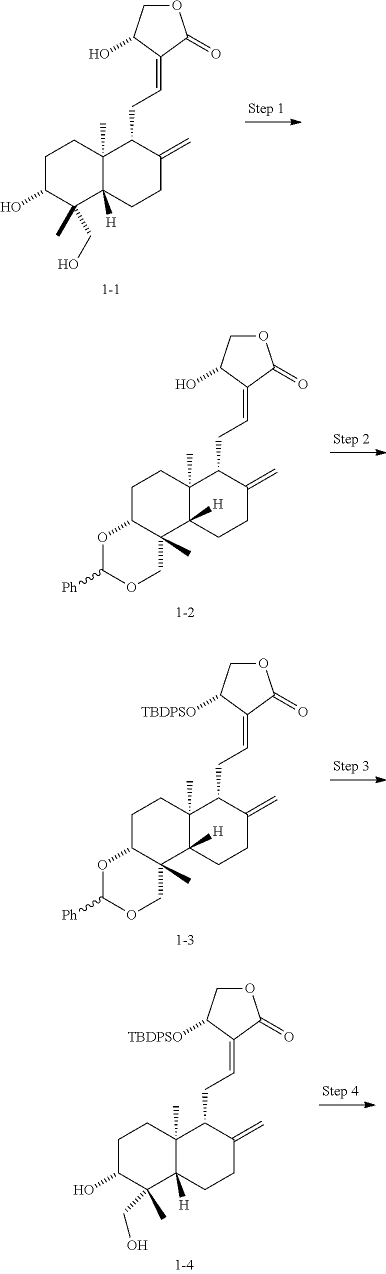

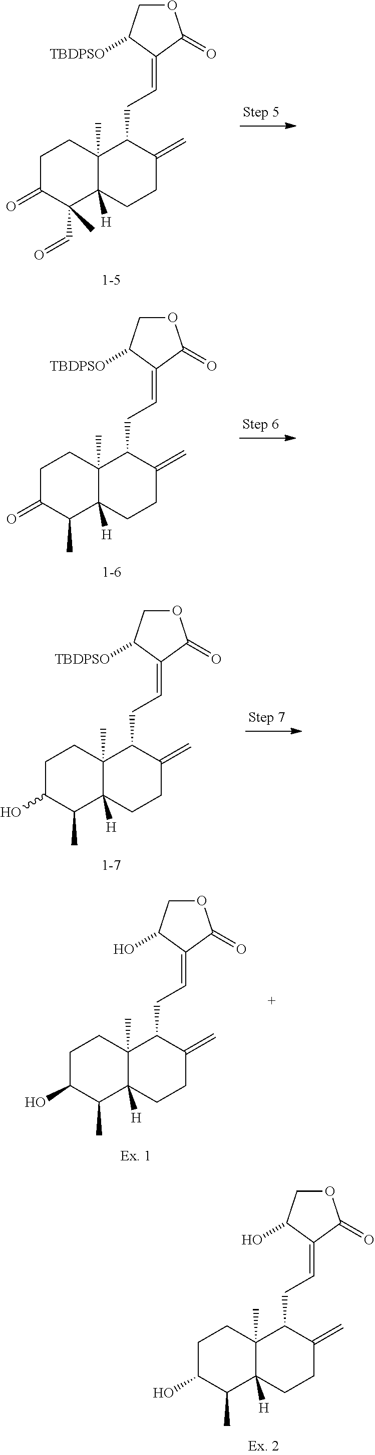

Step 1. To a mixture of commercially available compound 1-1 (17.5 g, 50 mmol, 1.0 eq.) and benzaldehyde dimethyl acetal (8.4 g, 55 mmol, 1.1 eq) in DCM (300 mL) at 5˜10° C., p-TsOH (1.0 g, 5 mmol, 10% eq.) was added and the mixture was stirred for 2 hat room temperature. TLC (PE: EtOAc=1:2) indicated that most of compound 1-1 was consumed. The mixture was washed with saturated NaHCO₃ solution (300 mL) and brine (200 mL), the organic layer was separated and concentrated in vacuo to afford compound 1-2 (21.0 g, crude) as a pale yellow solid, which was used for next step directly.

Step 2. To a solution of compound 1-2 (17.6 g, 40 mmol, 1.0 eq.) in dry DMF (500 mL) was added imidazole (27.6 g, 400 mmol, 10.0 eq) and followed by TBDPSCl (65.3 g, 240 mmol, 6.0 eq) dropwise at room temperature. The resulting mixture was stirred for 1 h at r.t. TLC (PE: EtOAc=2:1) indicated that about half of compound 1-2 was consumed. Water (1 L) and ethyl acetate (500 mL) were added to the mixture to quench the reaction, and the organic phase was separated and concentrated. The residue was purified by column chromatography on silica gel (PE: EtOAc=8:1) to afford compound 1-3 (10.1 g, 40%) as a white solid.

¹HNMR (400 MHz, CDCl₃) δ: 7.70 (s, 1H), 6.77-6.71 (m, 1H), 6.11 (d, J=15.6 Hz, 1H), 5.02 (d, J=11.6 Hz, 1H), 4.96 (d, J=1.2 Hz, 2H), 4.80 (d, J=1.2 Hz, 1H), 4.49 (d, J=1.2 Hz, 1H), 3.96 (dd, J=4.4Hz, J=12.8 Hz, 1H), 3.60 (d, J=10.4 Hz, 1H), 2.59˜2.51 (m, 1H), 2.45˜2.41 (m, 2H), 2.11˜2.02 (m, 2H), 1.79˜1.76 (m, 1H), 1.43˜1.32 (m, 6H),1.11˜1.04 (m, 1H), 0.87 (s, 3H).

Step 3. A suspension of compound 1-3 (10.1 g, 15 mmol, 1.0 eq.) in a mixture of solvents of HOAc (100 mL), H₂O (25 mL) and THF (25 mL) was heated to 70° C. for 5 h. TLC (PE: EtOAc=2:1) indicated that compound 1-3 was consumed. 100 mL of ice water was added to the mixture which was then extracted with EtOAc (2×50 mL). The combined organic phases were washed with saturated NaHCO₃ solution (50 mL), water (50 mL) and brine (100 mL). The organic phase was concentrated and the residue was purified by column chromatography (PE: EtOAc=1:2) to afford compound 1-4 (6.7 g, 80%) as a white solid.

¹HNMR (400 MHz, DMSO) δ: 7.63 (s, 1H), 6.76˜6.69 (m, 1H), 6.11 (d, J=16 Hz, 1H), 5.02 (d, J=4.8 Hz, 1H), 4.88 (s, 2H), 4.72 (s, 1H), 4.40 (s, 1H), 4.12 (dd, J=2.8 Hz, J=7.2 Hz, 1H), 3.82 (dd, J=2.8 Hz, J=10.8 Hz, 1H), 3.27˜3.18 (m, 2H), 2.34 (d, J=10.4 Hz, 2H), 1.99˜1.92 (m, 1H), 1.73˜1.60 (m, 2H), 1.57˜1.54 (m, 2H), 1.41˜1.33 (m, 2H), 1.20˜1.13 (m, 2H), 1.08 (s, 3H), 0.74 (s, 3H).

Step 4. To a solution of compound 1-4 (6.7 g, 11.37 mmol, 1.0 eq.) in DCM (70 mL) cooled in an ice bath was added Dess-Martin reagent (5.78 g, 13.65 mmol, 1.2 eq) in portions. The mixture was allowed to warm to r.t. and stirred for 2 h. TLC (PE : EA=1:1) indicated that compound 1-4 was consumed completely. The mixture was poured into a cooled saturated Na₂SO₃ aqueous solution (100 mL) and extracted with DCM (150 mL×3). The organic layer was concentrated in vacuum and the residue was purified by column chromatography (PE: EtOAC=7:1) to afford compound 1-5 (3.6 g, 53.7%) as a white solid.

¹H NMR (300 MHz, CDCl₃) δ: 9.58 (s, 1H), 7.70-7.55 (m, 4H), 7.48-7.29 (m, 6H), 6.75 (t, J=5.9 Hz, 1H), 4.91 (s, 2H), 4.50 (s, 1H), 4.14 (dd, J=2.0, 10.1 Hz, 1H), 3.95 (dd, J=5.7, 10.2 Hz, 1H), 2.55-2.37 (m, 2H), 2.34-2.14 (m, 2H), 2.13-1.99 (m, 1H), 1.95-1.80 (m, 2H), 1.75-1.38 (m, 5H), 1.18 (s, 3H), 0.97 (s, 9H), 0.61 (s, 3H).

Step 5. To a suspension of chloro(methoxymethyl)triphenylphosphorane (290 mg, 0.5 mmol) in THF (20 mL) at 0° C. was added LHMDS (1 mL, 1M in THF) in portions. The mixture was stirred for 15 min at 0° C. Compound 1-5 (290 mg, 0.5 mmol) was added and the mixture was warmed to room temperature and reaction was continued for 40 min. TLC (PE:EA=3:1) indicated that most of compound 1-5 was consumed. The mixture was poured into a saturated NH₄Cl solution (20 mL) slowly, and extracted with ethyl acetate (3×20 mL). The combined organic extracts were washed with brine (20 mL) and concentrated. The residue was purified by column chromatography on silica gel (PE: EtOAc=6:1-2:1) to afford the compound 1-6 (60 mg, 21.5%) as a white solid. ¹H NMR (400 MHz, CDCl₃) δ: 7.76-7.64 (m, 4H), 7.53-7.35 (m,6H), 6.94-6.76 (m, 1H), 5.05-4.88 (m, 2H), 4.62-4.51 (s, 1H), 4.22-4.16 (m, 1H), 4.04-3.97 (m, 1H), 2.51-2.10 (m, 5H), 1.99-1.87 (m, 1H), 1.80-1.60 (m, 2H), 1.57-1.43 (m, 1H), 1.37-1.15 (m, 5H), 1.10-1.02 (m, 9H), 0.98 (d, J=6.5 Hz, 2H), 0.83-0.64 (m, 3H)

Step 6. To a solution of compound 1-6 (660 mg, 1.18 mmol, 1.0 eq.) in MeOH (10 mL) was added NaBH₃CN (594 g, 9.42 mmol, 9.4 eq) and AcOH (0.2 mL). The mixture was stirred for 12 hours. TLC (PE:EA=3:1) indicated that compound 1-6 was consumed completely. The mixture was poured into water (30 mL) and extracted with EA (50 mL×3). The organic layer was removed in vacuum and the residue was purified by column chromatography on silica gel (PE: EtOAC=6:1) to afford compound 1-7 (434 mg, 65.7% yield) as a white solid.

¹H NMR (300 MHz, CDCl₃) δ: 7.75-7.64 (m, 4H), 7.50-7.34 (m, 6H), 6.95-6.78 (m, 1H), 5.05-4.94 (m, 1H), 4.89 (s, 1H), 4.50 (s, 1H), 4.23-4.12 (m, 1H), 4.00-3.92 (m, 1H), 3.22-3.00 (m, 1H), 2.44-2.15 (m, 5H), 2.00-1.83 (m, 1H), 1.81-1.58 (m, 2H), 1.47-1.35 (m, 1H), 1.33-1.16 (m, 3H), 1.05 (s, 9H), 0.97-0.94 (d, J=8 Hz, 3H), 0.54 (s, 3H).

Step 7. A solution of compound 1-7 (400 mg, 0.71 mmol, 1.0 eq.) in a complex of HF/Pyridine (10mL) and THF (40 mL) was stirred for 14 h at r.t. TLC indicated that most of compound 1-7 was consumed. The mixture was poured into a saturated NaHCO₃ aqueous solution (100 mL) slowly, stirred overnight and extracted with ethyl acetate (4×150 mL). The combined organic extracts were washed with brine (300 mL) and concentrated. The residue was purified by prep-HPLC (Mobile phase A: water with 0.1% formic acid, Mobile phase B: acetonitrile; Column: Gemini 250×20 mm×5 um; Detection wavelength: 220 nm) to afford Ex. 1 (17 mg, 7% yield) and Ex. 2 (73.1 mg, 32.2% yield) as colorless solid. Ex.1: ¹H NMR (400 MHz, CDCl₃) δ: 7.07-6.97 (m, 1H), 5.09-4.99 (m, 1H), 4.92 (s, 1H), 4.60 (s, 1H), 4.48-4.37 (m, 1H), 4.26 (dd, J=1.5, 10.5 Hz, 1H), 3.78 (s., 1H), 2.71-2.61 (m, 1H), 2.58-2.35 (m, 3H), 2.08-1.94 (m, 2H), 1.80-1.56 (m, 6H), 1.17-1.05 (m, 1H), 0.94 (d, J=6.0 Hz, 3H), 0.70 (s, 3H); HPLC Purity:99%;

LCMS: M+1=321.2. Ex.2: ¹H NMR (400 MHz, CDCl₃) δ: 7.08-6.97 (m, 1H), 5.05 (t, J=6.1 Hz, 1H), 4.93 (s, 1H), 4.61 (s, 1H), 4.46 (dd, J=6.0, 10.5 Hz, 1H), 4.27 (dd, J=2.0, 10.5 Hz, 1H), 3.15 (br. s., 1H), 2.69-2.49 (m, 2H), 2.44-2.36 (m, 1H), 2.04-1.75 (m, 6H), 1.54-1.44 (m, 2H), 1.35-1.01 (m, 4H), 0.99 (d, J=6.3 Hz, 3H), 0.74 (s, 3H); HPLC purity: 98.1%; LCMS: M+1=321.3.

### Example 3

### Synthesis of (S,E)-4-hydroxy-3-(2-((1R,4aR,5R,6R,8aR)-6-hydroxy-2,5,8a-trimethyl-1,4,4a,5,6,7,8,8a-octahydronaphthalen-1-yl)ethylidene)dihydrofuran-2(3H)-one (Ex.3)

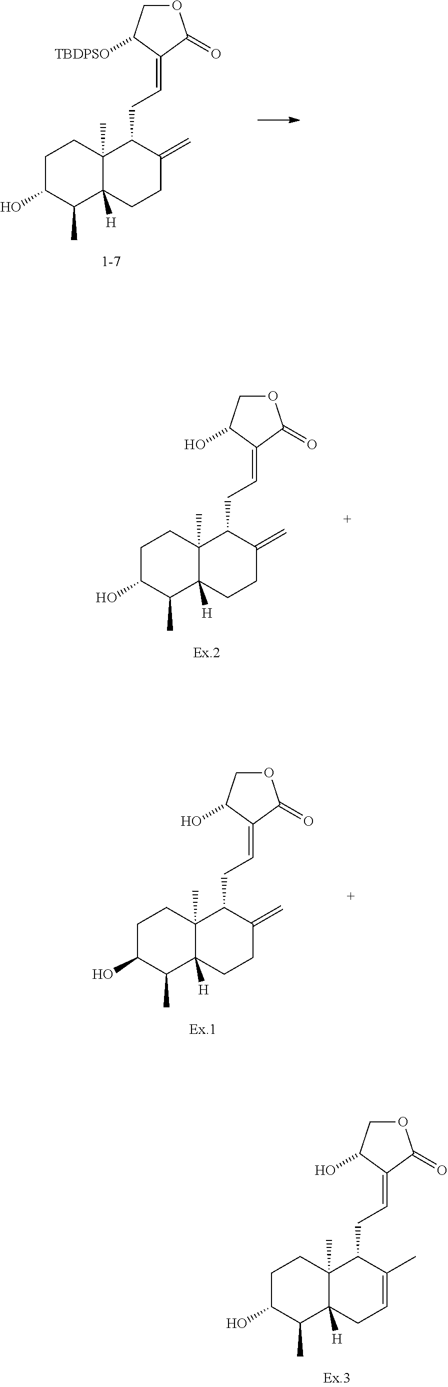

Example 3 can be prepared as a minor product from intermediate 1-7 available from Preparative Examples 1 and 2: a solution of 1-7 (1.98 g, 3.55 mmol, 1.0 eq.) in THF (20 mL) was added a complex solution of HF/Pyr (20 mL) dropwise at 0° C. The mixture was stirred at room temperature for 14 h. The reaction mixture was poured into cooled water carefully then extracted with EA (50 mL×3). The combined organic phase was washed with brine (30 mL×3) and dried over anhydrous sodium sulfate, filtered and concentrated. The residue was purified by column chromatography on silica gel eluted with PE: EA (1:1-3:1) to give a product mixture of Ex. 2, Ex.1 and Ex. 3 (0.9 g) as a white solid. Combining this material with a previously obtained batch, totaling 1.1 g product mixture was further purified by pre-HPLC (Mobile phase A: water with 0.1% formic acid, Mobile phase B: acetonitrile; Column: Gemini 250×20 mm×5 um; Detection wavelength: 220 nm) and then by SFC separation to afford Ex.2 (253.1 mg, 22.3%), Ex.3 (53 mg, 4.7%) and Ex.1 (40 mg, 3.5%) as white solids.

Ex.3: ¹H NMR (400 MHz, CDCl₃) δ: 7.13 (t, J=6.3 Hz, 1H), 5.49 (br. s., 1H), 5.06 (d, J=4.8 Hz, 1H), 4.48 (dd, J=6.1, 10.4 Hz, 1H), 4.27 (dd, J=1.6, 10.4 Hz, 1H), 3.14 (m, 1H), 2.65-2.57 (m, 2H), 2.19-2.01 (m, 2H), 1.90-1.80 (m, 2H), 1.68 (s, 3H), 1.64-1.41 (m, 4H), 1.34-1.22 (m, 1H), 1.20-1.04 (m, 2H), 0.99 (d, J=6.3 Hz, 3H), 0.79 (s, 3H); HPLC Purity: 99.0%; LCMS: M+H=321.0.

### Examples 4 and 5

### Synthesis of (S,E)-4-hydroxy-3-(2-((1R,4aR,5R,6R,8aR)-6-hydroxy-5,6,8a-trimethyl-2-methylenedecahydronaphthalen-1-yl)ethylidene)dihydrofuran-2(3H)-one (Ex.4)

### and (S,E)-4-hydroxy-3-(2-((1R,4aR,5R,6S,8aR)-6-hydroxy-5,6,8a-trimethyl-2-methylenedecahydronaphthalen-1-yl)ethylidene)dihydrofuran-2(3H)-one (Ex.5)

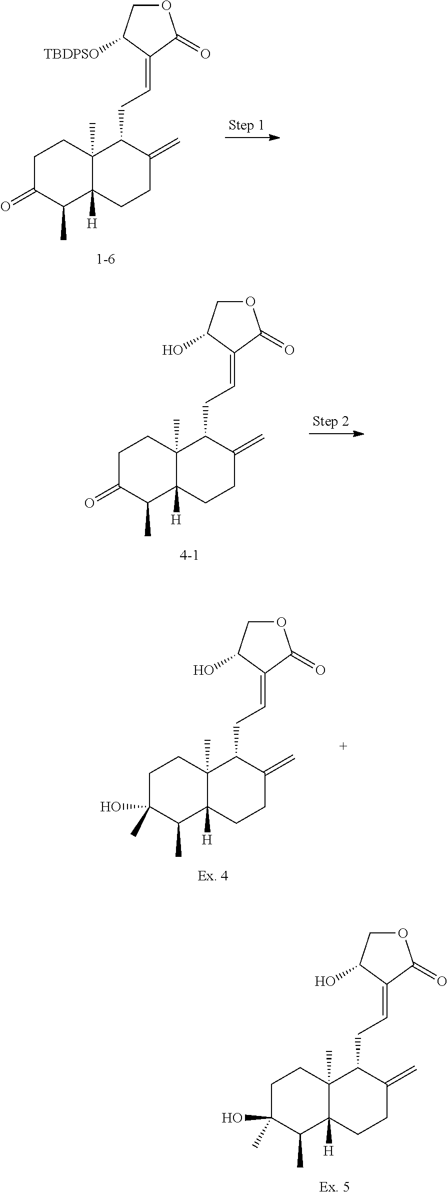

Step 1. To a solution of intermediate 1-6 available from Preparative Examples 1 and 2 (0.7 g, 1.25 mmol, 1.0 eq.) in THF (7 mL) was added HF/Pyr complex solution (7 mL) dropwise at 0° C. The mixture was stirred at room temperature for 16 h. The reaction mixture was poured into cooled water carefully and then extracted with EA (10 mL×3). The combined organic phase was washed with brine (20 mL×3) and dried over anhydrous sodium sulfate, filtered and concentrated. The residue was purified by column chromatography on silica gel with elution PE: EA (1: 1) to give intermediate 4-1 (394 mg, 99%) as white solid.

¹HNMR (400 MHz, CDCl₃) δ: 6.98 (t, J=6.7 Hz, 1H), 5.04 (br. s., 1H), 5.00 (s, 1H), 4.69 (s, 1H), 4.48 (dd, J=6.1, 10.4 Hz, 1H), 4.27 (dd, J=1.5, 10.5 Hz, 1H), 2.66 (t, J=7.0 Hz, 2H), 2.59-2.36 (m, 4H), 2.29 (m, 1H), 2.14-2.05 (m, 1H), 2.04-1.93 (m, 2H), 1.84 (d, J=13.1 Hz, 1H), 1.58 (m, 1H), 1.45 (m, 1H), 1.36-1.21 (m, 1H), 1.01 (d, J=6.3 Hz, 3H), 0.98 (s, 3H).

Step 2. To a solution of intermediate 4-1 (0.4 g, 1.26 mmol, 1.0 eq.) in THF (6 mL) was added a 1M solution of MeLi in THF (4.4 mL, 4.4 mmol, 3.5 eq.) at −78° C. dropwise. The resulting suspension was stirred at this temperature for 10 min and then quenched with a saturated NH₄Cl aqueous solution (10 mL). The aqueous layer was extracted with EA (15 mL×3). The combined organic extracts were washed with water (30 mL×3), brine (30 mL×3), dried over anhydrous sodium sulfate, filtered and concentrated.

The residue was purified by preparative TLC with PE: EA=1:2 to give the crude products of Ex.4 (40 mg) and Ex. 5 (194 mg) respectively.

Ex.4 was further purified by preparative HPLC and SFC to afford pure Ex.4 (10.8 mg, 2.5%) as a white solid. ¹HNMR (400 MHz, CDCl3) δ: 7.03 (t, J=6.7 Hz, 1H), 5.06 (d, J=5.8 Hz, 1H), 4.94 (s, 1H), 4.62 (s, 1H), 4.48 (dd, J=6.0, 10.5 Hz, 1H), 4.28 (dd, J=1.9, 10.4 Hz, 1H), 2.69-2.51 (m, 2H), 2.41 (d, J=12.0 Hz, 1H), 2.06-1.89 (m, 2H), 1.78-1.65 (m, 6H), 1.55-1.45 (m, 1H), 1.32-1.24 (m, 1H), 1.18-1.09 (m, 5H), 0.89 (d, J=6.5 Hz, 3H), 0.76 (s, 3H); HPLC purity: 100%; LCMS: M−OH=317.0.

Ex.5 was further purified by preparative HPLC (Mobile phase A: water with 0.1% formic acid, Mobile phase B: acetonitrile; Column: Gemini 250×20 mm×5 um; Detection wavelength: 220 nm) to give the final product (79.3 mg, 18.8%) as a white solid. ¹HNMR (400 MHz, CDCl₃) δ: 7.09-7.00 (m, 1H), 5.05 (d, J=5.5 Hz, 1H), 4.93 (s, 1H), 4.60 (s, 1H), 4.46 (dd, J=5.9, 10.4 Hz, 1H), 4.27 (d, J=10.5 Hz, 1H), 2.72-2.61 (m, 1H), 2.57-2.46 (m, 1H), 2.40 (d, J=12.8 Hz, 1H), 2.09-1.93 (m, 2H), 1.79 (d, J=13.3 Hz, 1H), 1.67-1.50 (m, 6H), 1.45 (m, 1H), 1.38-1.28 (m, 1H), 1.22 (s, 3H), 1.09 (dq, J=4.3, 12.9 Hz, 1H), 0.91 (d, J=6.8 Hz, 3H), 0.70 (s, 3H); HPLC purity: 100%; LCMS: M−OH=317.0.

### Examples 6 and 7

### Synthesis of (S,E)-4-hydroxy-3-(2-((1R,4aS,5R,6S,8a5)-6-hydroxy-5-(hydroxymethyl)-5,6,8a-trimethyl-2-methylenedecahydronaphthalen-1-yl)ethylidene)dihydrofuran-2(3H)-one (Ex. 6)

### and (S,E)-4-hydroxy-3-(2-((1R,4aS,5R,6R,8a5)-6-hydroxy-5-(hydroxymethyl)-5,6,8a-trimethyl-2-methylenedecahydronaphthalen-1-yl)ethylidene)dihydrofuran-2(3H)-one (Ex.7)

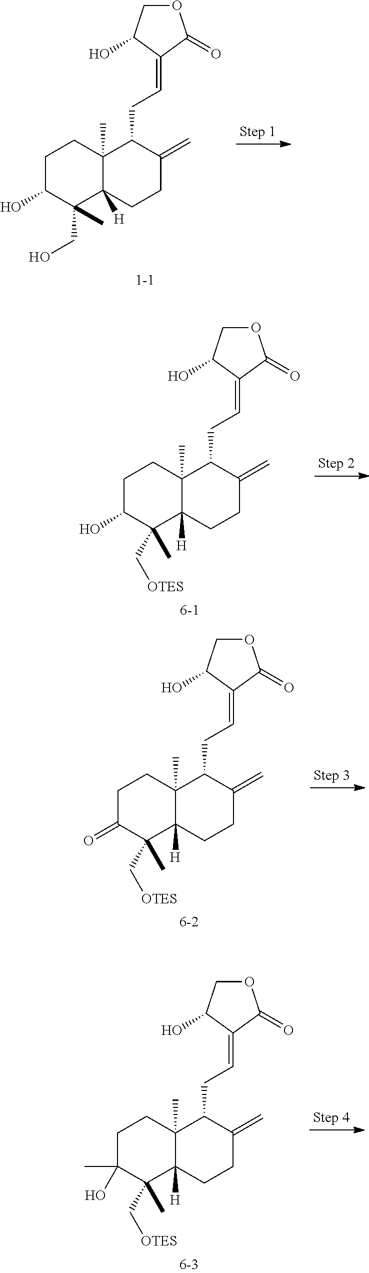

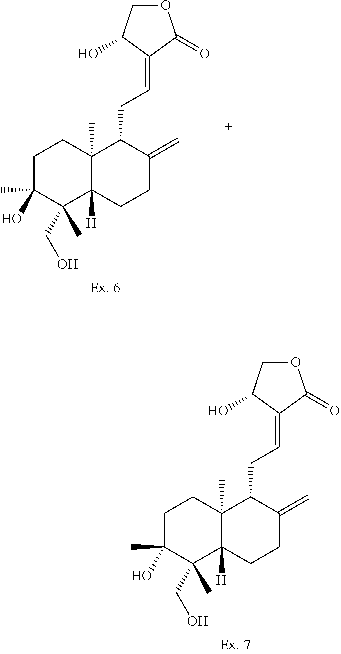 [SMILES: C=C1CC[C@H]2[C@@](C)(CC[C@](C)(O)[C@@]2(C)CO)[C@@H]1C/C=C1/C(=O)OC[C@H]1O ; C=C1CC[C@H]2[C@@](C)(CC[C@@](C)(O)[C@@]2(C)CO)[C@@H]1C/C=C1/C(=O)OC[C@H]1O]

Step 1. To a solution of compound 1-1 (50 g, 143 mmol, 1.0 eq.) in anhydrous DCM (500 mL) cooled in an ice bath was added TEA (28.9 g, 286 mmol, 2.0 eq.) and followed by dropwise addition of TESCl (22.5 g, 150 mmol, 1.05 eq.). The reaction mixture was stirred at room temperature for 10-20 min. TLC showed there was still about 5% starting material remaining in the reaction mixture. The mixture was poured into 500 mL of water and extracted with DCM (300 mL×3). The combined organic extracts were washed with brine, dried over anhydrous Na₂SO₄ and concentrated to give the crude product of 6-2 (50 g), which was used in the next step without further purification.

Step 2. To a solution of compound 6-2 (50 g, 143 mmol, 1.0 eq.) in anhydrous DCM (600 mL) cooled in an ice bath was added Dess-Martin reagent (72.7 g, 170 mmol, 1.2 eq.) in small portions. After addition, the reaction mixture was stirred at room temperature for 2 h. TLC showed there was still about 20% starting material in the mixture. Additional portion of Dess-Martin reagent (18 g, 40 mmol, 0.3 eq.) was added and reaction was continued at room temperature for 1 h. The mixture was poured into 400 mL of a saturated Na₂SO₃ aqueous solution and extracted with DCM (500 mL×3). The combined organic layers were washed with brine, dried over anhydrous Na₂SO₄ and concentrated to give the crude product, which was purified by column chromatography on silica gel eluted with PE: EtOAc=10:1 to DCM: EtOAc=2:1 to give the compound 6-2 (25 g, 38% yield for two steps). ¹HNMR (400 MHz, CDCl₃) δ: 6.95 (t, J=6.4 Hz, 1H), 5.02 (d, J=4.6 Hz, 1H), 4.98 (s, 1H), 4.65 (s, 1H), 4.45 (dd, J=6.2, J=10.2 Hz, 1H), 4.25 (d, J=10.4 Hz, 1H), 3.82 (d, J=9.8 Hz, 1H), 3.49 (d, J=9.8 Hz, 1H), 2.73-2.52 (m, 3H), 2.50-2.29 (m, 2H), 1.91 (d, J=6.2 Hz, 1H), 1.75 (d, J=10.6 Hz, 1H), 1.60-1.46 (m, 2H), 1.08 (s, 3H), 0.99 (s, 3H), 0.89 (t, J=7.8 Hz, 9H), 0.59-0.43 (m, 6H).

Step 3. To a solution of compound 6-2 (0.9 g, 1.95 mmol, 1.0 eq.) in anhydrous THF (45.0 mL) at −78° C. under N₂ was added dropwise a 1.0 M solution of MeLi in 2-methyltetrahydrofuran (11.7 mL, 11.7 mmol). The reaction mixture was stirred for 0.5 h. A saturated NH₄Cl aqueous solution was added to quench the reaction at −78° C. The two phases were separated, and the aqueous phase was extracted with EtOAc (50 mL×3). The combined organic phase was dried over Na₂SO₄, filtered, concentrated in vacuo to give a crude product, which was purified by column chromatography to give a crude compound 6-3 (200 mg, 21.5%) as a white solid, which was used directly in the next step.

Step 4. To a solution of compound 6-3 (500 mg, 1.02 mmol, 1.0 eq.) in 10 mL of a mixed solvent system of THF: H₂O (4:1, v/v) was added a 2 M aqueous HCl solution (1.0 mL, 2.0 mmol, 2.0 eq.) at room temperature. The reaction mixture was stirred for 10 min at r.t. TLC (PE/EA=1:1) showed the starting material was consumed. A saturated NaHCO₃ aqueous solution was added to quench the reaction. The two phases were separated, and the aqueous phase was extracted with EtOAc (10 mL×3). The combined organic pahse was washed with brine, dried over Na₂SO₄, filtered, concentrated in vacuo to give a crude product, which was purified by column chromatography to give a mixture of the two titled products (300 mg, 80.9%, about 85% purity). 100 mg of this mixture of products was further purified by prep-HPLC (Mobile phase A: water with 0.1% formic acid, Mobile phase B: acetonitrile; Column: DIKMA Diamonsil C18 200×25×5 um wavelength: 220 nm) to provide Ex.6 (15.0 mg) and Ex.7 (22.1 mg) as colorless solids.

Ex.6: ¹HNMR (400 MHz, CD3OD) δ: 6.90-6.86 (m, 1H), 5.03-5.01 (m, 1H), 4.93 (s, 1H), 4.65 (s, 1H), 4.48-4.46 (m, 1H), 4.18-4.15 (m, 1H), 3.59-3.49 (m, 2H), 2.64-2.40 (m, 3H), 2.00-1.83 (m, 5H), 1.54-1.47 (m, 4H), 1.22 (s, 3H), 1.02 (s, 3H), 0.73 (s, 3H); HPLC Purity: 99.8%; LCMS: M+Na=387.0.

Ex.7: ¹HNMR (400 MHz, CD3OD) δ: 6.88-6.84 (m, 1H), 5.02 (m, 1H), 4.99 (s, 1H), 4.67 (s, 1H), 4.49-4.45 (s, 1H), 4.23-4.15 (m, 2H), 3.35 (m, 1H), 2.62-2.41 (m, 3H), 2.00-1.86 (m, 3H), 1.73-1.40 (m, 6H), 1.27 (s, 3H), 1.08 (s, 3H), 0.75 (s, 3H); HPLC Purity: 99.8%; LCMS: M+Na=387.1.

### Examples 8 and 9

Synthesis of (S,E)-3-(2-((3S, 4aR, 6aS, 7R, 10aS, 10bR)-6a,10b-dimethyl-8-methylene-3-oxidodecahydro-1H-naphtho[2,1-d][1,3,2]dioxathiin-7-yl)ethylidene)-4-hydroxydihydrofuran-2(3H)-one (Ex.8) and (S,E)-3-(2-((3R,4aR,6aS,7R,10aS,10bR)-6a,10b-dimethyl-8-methylene-3-oxidodecahydro-1H-naphtho[2,1-d][1,3,2]dioxathiin-7-yl)ethylidene)-4-hydroxydihydrofuran-2(3H)-one (Ex.9).

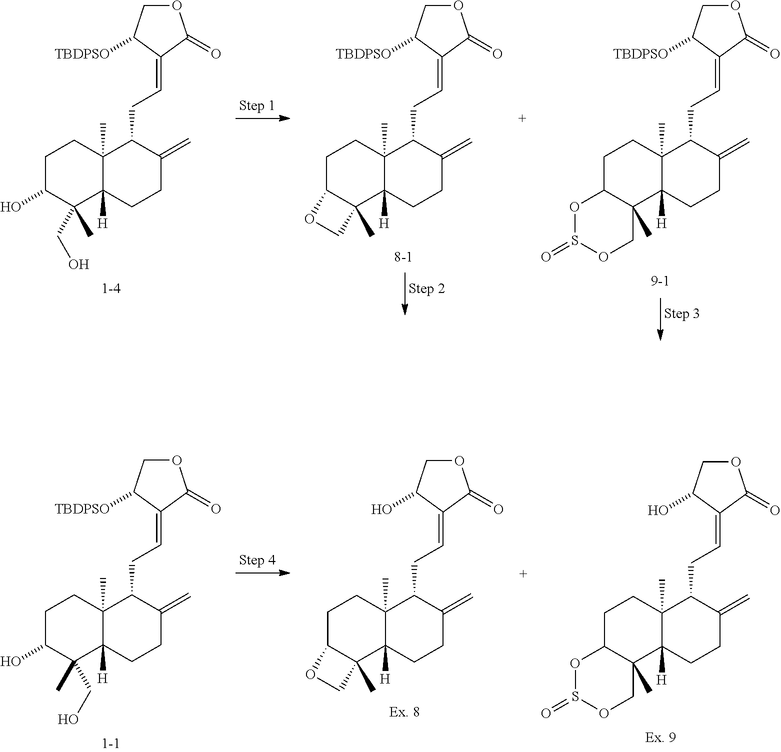

Step 1. To a solution of intermediate 1-4 (1.2 g, 2 mmol, 1.0 eq.), available from Preparative Example 1, in 12 mL of THF in an ice bath, was added dropwise a solution of SOCl₂ (0.5 g, 4 mmol, 2.0 eq) in THF (3 mL). The mixture was allowed to warm to room temperature and stirred for 1 h. TLC (PE: EtOAC=2:1) indicated that compound 1-4 was consumed completely. Solvent was removed in vacuum and the residue was purified by column chromatography on silica gel (PE: EtOAC=5:1) to afford compound 8-1 (0.4 g, 35%) and compound 9-1 (0.57 g, 35%) as colorless solids.

¹HNMR for compound 8-1(400 MHz, CDCl₃) δ: 7.70˜7.68 (m, 4H), 7.49˜7.39 (m, 6H), 6.83˜6.79 (m, 4H), 5.13˜5.10 (d, J=11 Hz, 1H), 4.97˜4.96 (d, J=11 Hz, 1H), 4.90 (s, 1H), 4.51 (s, 1H), 4.21˜4.18 (dd, J=10.3, 2 Hz, 1H), 4.02˜3.98 (d, J=10.7 Hz, 1H), 3.75˜3.70 (dd, J=12.7, 4.4 Hz, 1H), 3.40˜3.37 (d, J=11.5 Hz, 1H), 2.77˜2.65 (m, 1H),2.44˜2.41 (d, J=12.3Hz, 1H), 2.30˜2.22 (m, 1H), 2.14˜2.08 (m, 2H),1.96˜1.90 (m, 1H), 1.79˜1.77 (m, 1H), 1.40 (s, 3H), 1.34˜1.16 (m, 3H), 1.05 (s, 9H), 0.80˜0.73 (m, 1H), 0.65 (s, 3H).

¹HNMR for compound 9-1(400 MHz, CDCl₃) δ: 7.64-7.60 (m, 4H), 7.44-7.32 (m, 6H), 6.76-6.72 (t, J=5.9 x 2, 1H), 4.92 (d, J=5.3Hz, 1H), 4.84 (s, 1H), 4.54-4.46 (m, 2H), 4.12 (m, 1H), 3.95-3.90 (dd, J=10.2, 5.7, 2H), 3.78-3.74 (d, J=12.2, 1H), 2.37-1.99 (m, 5H), 1.95-1.81 (m, 2H), 1.72-1.69 (m, 1H), 1.59 (s, 1H), 1.35 (s, 3H), 1.30-1.27 (m, 2H), 0.98 (m, 9H), 0.92-0.77 (m, 1H), 0.57 (s, 3H).

Step 2. A solution of compound 8-1 (571 mg, 1 mmol, 1.0 eq.) in a mixture of HF/Py (2 mL) and CH₃CN (10 mL) was stirred at room temperature for 8 h. TLC (PE: EtOAC=1: 1) indicated that most of starting material was consumed. The mixture was poured into a saturated NaHCO₃ aqueous solution (10 mL) slowly, and extracted with ethyl acetate (3 x 10 mL). The combined organic phase was washed with brine and then concentrated. The residue was purified by pre-HPLC to afford the titled compound Ex.8 (111 mg, 33%) as a colorless solid. Ex.8: ¹HNMR (400 MHz, CDCl₃) δ: 6.95˜6.92 (m, 1H), 5.17˜5.15 (d, J=11.3 Hz, 1H), 5.05˜5.04 (d, J=5.5 Hz, 1H), 4.94 (s, 1H), 4.64 (s, 1H), 4.49˜4.45 (dd, J=10.5, 6.3 Hz, 1H), 4.28˜4.25 (m, 1H), 3.83˜3.79 (dd, J=13.1, 4.0 Hz, 1H), 3.43˜3.40 (d, J=11.5 Hz, 1H), 2.88˜2.77 (m, 1H), 2.58˜2.46 (m, 3H), 2.26˜2.21 (m, 1H), 2.04˜1.98 (m, 1H), 1.87˜1.84 (m, 3H), 1.44 (s, 3H), 1.37˜1.25 (m, 3H), 1.10˜1.04 (m, 1H), 0.87 (s, 3H); HPLC purity 100%; LCMS: [M−OH]+=379.3. The structure was confirmed by a single crystal x-ray structure.

Step 3. A solution of compound 9-1 (634 mg, 1 mmol, 1.0 eq.) in a mixture of HF/Py (10 mL) and THF (40 mL) was stirred for 6 h at r.t. TLC (PE: EtOAC=1:1) indicated that most of the starting material was consumed. The mixture was poured into a saturated NaHCO₃ aqueous solution (100 mL) slowly, and extracted with ethyl acetate (3×20 mL). The combined organic extracts were washed with brine (200 mL) and then concentrated. The residue was purified by silicon column (PE: EtOAC=6:1-2:1) to afford the titled product Ex.9 (300 mg, 75%) as a colorless solid. Ex.9: ¹HNMR (400 MHz, DMSO-d6) δ: 7.63 (s, 1H), 6.76˜6.69 (m, 1H), 6.11 (d, J=16 Hz, 1H), 5.02 (d, J=4.8 Hz, 1H), 4.88 (s, 2H), 4.72 (s, 1H), 4.40 (s, 1H), 4.12 (dd, J=2.8 Hz, J=7.2 Hz, 1H), 3.82 (dd, J=2.8 Hz, J=10.8 Hz, 1H), 3.27˜3.18 (m, 2H), 2.34 (d, J=10.4 Hz, 2H), 1.99-1.92 (m, 1H), 1.73˜1.60 (m, 2H), 1.57˜1.54 (m, 2H), 1.41˜1.33 (m, 2H), 1.20˜1.13 (m, 2H), 1.08 (s, 3H), 0.74 (s, 3H); HPLC Purity: 98.4%; LC-MS: M+1: 315.1; MS: M+H=397. The structure was confirmed by a single crystal x-ray structure

Step 4. Alternatively the titled compounds Ex.8 and Ex.9 can be prepared directly from compound 1-1. To a suspension of compound 1-1 (5 g, 14.3 mmol, 1.0 eq.) in anhydrous THF (50 mL) was added SOCl₂(2.04 g, 17.1 mmol, 1.2 eq.) in 10 mL anhydrous THF dropwise at −20° C. The reaction mixture was stirred at -20° C. for 20 min. TLC (PE: EA=1: 2) showed it was still staring material. Reaction was continued in an ice bath for additional 20 min. TLC (PE: EA=1: 2) showed most of the starting material was consumed. A saturated aqueous NaHCO₃ solution was added slowly to the reaction mixture at 0° C. and the aqueous mixture was extracted with DCM/MeOH (100 mL×4). The combined organic layers were washed with brine, dried over anhydrous Na₂SO₄ and concentrated in vacuum. The residue was purified by silica gel chromatography eluting with PE: EtOAc=5:1 to DCM: MeOH=20: 1 to afford a mixture of the titled products Ex.8 and Ex.9 (4.05 g, semi crude). The two products were re-purified by preparative HPLC (Mobile phase A: water with 0.1% formic acid, Mobile phase B: acetonitrile; Column: Agela DuraShell 200×25 mm×5 um; Detection wavelength: 220 nm) to give desired pure compound Ex.8 (280 mg) as a white solid.

### Example 10

### Synthesis of (R,E)-3-(2-((3S,4aR,6aS,7 R,10aS,10bR)-6a,10b-dimethyl-8-methylene-3-oxidodecahydro-1H-naphtho[2,1-d][1,3,2]dioxathiin-7-yl)ethylidene)-4-hydroxydihydrofuran-2(3H)-one

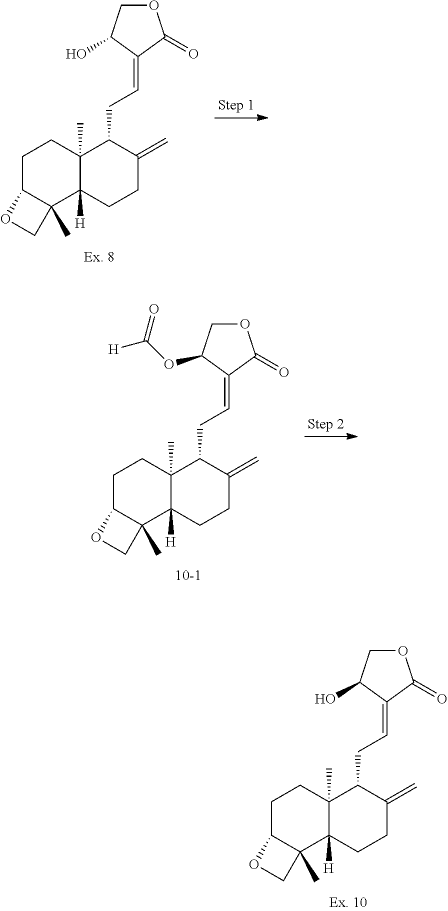 [SMILES: C=C1CC[C@@H]2[C@]3(C)CO[C@@H]3CC[C@@]2(C)[C@@H]1C/C=C1/C(=O)OC[C@H]1O ; C=C1CC[C@@H]2[C@]3(C)CO[C@@H]3CC[C@@]2(C)[C@@H]1C/C=C1/C(=O)OC[C@@H]1OC=O ; C=C1CC[C@@H]2[C@]3(C)CO[C@@H]3CC[C@@]2(C)[C@@H]1C/C=C1/C(=O)OC[C@@H]1O]

Step 1. To a mixture of Ex.8 (contaminated by Ex.9 in a ratio of 1 to 4, mol/mol, total 1.6 g, 4 mmol, 1.0 eq.), PPh₃ (1.57 g, 6 mmol, 1.5 eq.) and formic acid (0.368 g, 8 mmol, 2 eq.) in dry THF (30 mL) was added DIAD (1.2 g, 6 mmol, 1.5 eq.) dropwise under a N₂ atmosphere in an ice bath. Then the reaction mixture was stirred at room temperature for 3 hour. TLC (PE: EA=2:1) showed the staring material was consumed. The reaction mixture was directly purified by silica gel chromatography eluted with PE: EtOAc=8:1 to PE: EtOAc=3:1 to give a crude product of compound 10-1 (2.7 g, white solid, mixed with PPO and diisopropyl hydrazine-1,2-dicarboxylate). The crude product was used in the next step directly. ¹HNMR (400 MHz, CDCl₃) δ: 8.15 (s, 1H), 7.02 (t, J=6.3 Hz, 1H), 6.08 (d, J=6.3 Hz, 1H), 5.20-5.14 (d, J=10.3 Hz, 1H), 4.92 (s, 1H), 4.62 (dd, J=6.1, 11.4 Hz, 1H), 4.42 (s, 1H), 4.30 (d, J=10.3 Hz, 1H), 3.83 (dd, J=4.5, 12.5 Hz, 1H), 3.43 (d, J=11.3 Hz, 1H), 2.84 (dq, J=3.5, 13.6 Hz, 1H), 2.53-2.36 (m, 2H), 2.29-2.20 (m, 2H), 1.94-1.78 (m, 4H), 1.46 (s, 3H), 1.38 (d, J=6.3 Hz, 2H), 1.11-1.02 (m, 1H), 0.87 (s, 3H).

Step 2. To a solution of the crude compound 10-1 (2.7 g, ˜1 mmol, 1.0 eq.) in MeOH (10 mL) and THF (10 mL) was added NaHCO₃ (0.84 g, 10 mmol, 10 eq.) and the reaction mixture was stirred at room temperature for 3 h. TLC (PE: EA=1:1) indicated that half of the started material was consumed. Additional NaHCO₃ (0.84 g, 10 mmol, 10 eq) was added and stirring was continued for another 3 hour. The reaction mixture was filtered, and the filtrate was concentrated and then purified by column chromatography on silica gel eluted with PE: EA=5:1 to 2:1 to give the titled product (650 mg) which was still mixed with triphenylphosphino oxide (PPO). Further purification was done by prep-TLC (PE: EA=2:3) to afford the product Ex.10 (96 mg, 29% yield over 2 steps) as a white solid. ¹HNMR (400 MHz, CDCl₃) δ: 6.97-6.90 (m, 1H), 5.16 (d, J=11.3 Hz, 1H), 5.08 (d, J=5.3 Hz, 1H), 4.90 (s, 1H), 4.48 (dd, J=6.3, 10.5 Hz, 1H), 4.45 (s, 1H), 4.25 (dd, J=2.0, 10.5 Hz, 1H), 3.82 (dd, J=4.3, 13.6 Hz, 1H), 3.42 (d, J=11.3 Hz, 1H), 2.90-2.76 (m, 1H), 2.66 (dd, J=5.4, 14.9 Hz, 1H), 2.50-2.38 (m, 2H), 2.28-2.19 (m, 1H), 2.05-1.96 (m, 1H), 1.94-1.81 (m, 4H), 1.44 (s, 3H), 1.38-1.25 (m, 2H), 1.18-1.07 (m, 1H), 0.88 (s, 3H); HPLC Purity: 97.35%; LCMS: [M−OH]+=379.1.

### Example 11

### Synthesis of (S,E)-3-(2-((5aS,6R,9aS,9bS)-5a,9b-dimethyl-7-methylene-1,4,5,5a,6,7,8,9,9a,9b-decahydronaphtho[2,1-c]isoxazol-6-yl)ethylidene)-4-hydroxydihydrofuran-2(3H)-one

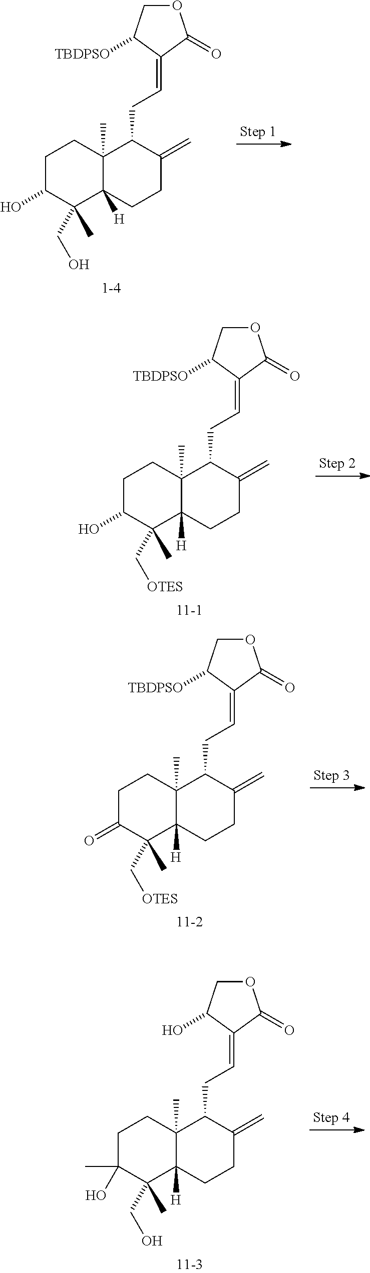

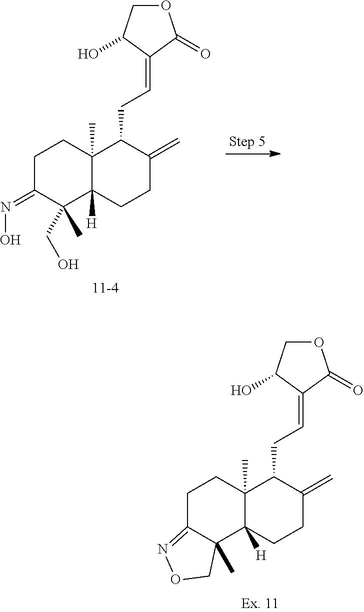 [SMILES: C=C1CC[C@H]2[C@@](C)(CC/C(=N/O)[C@@]2(C)CO)[C@@H]1C/C=C1/C(=O)OC[C@H]1O ; C=C1CC[C@H]2[C@@](C)(CCC3=NOC[C@]32C)[C@@H]1C/C=C1/C(=O)OC[C@H]1O]

Step 1. To a solution of intermediate 1-4 (20 g, 34 mmol, 1.0 eq.), available from Preparative Example 1, in anhydrous DCM (167 mL) and MeCN (167 mL) was added TEA (6.9 g, 68 mmol, 2.0 eq.) and TESCl (6.6 g, 44.2 mmol, 1.3 eq.) dropwise in an ice bath. Cooling bath was removed; the reaction was continued at 30° C. for 1.5 h. TLC showed there was no starting material remaining in the mixture. The reaction mixture was poured into 500 mL water, slowly adjusted pH to 7 using a 1N HCl aq. solution, and then extracted with DCM (3×300 mL). The combined organic layers were washed with brine, dried over anhydrous Na₂SO₄ and concentrated to give a crude product, which was purified by column chromatography on silica gel with PE: EtOAc (20:1) to PE: EtOAc (5:1) to provide compound 11-1 (15 g, 63% yield) as a yellow solid. ¹HNMR (400 MHz, CDCl₃) δ: 7.69 (d, J=7.6 Hz, 4H), 7.51-7.38 (m, 6H), 6.89-6.80 (m, 1H), 4.97 (d, J=4.0 Hz, 1H), 4.85 (s, 1H), 4.53-4.42 (m, 2H), 4.22-4.13 (m, 2H), 3.98 (dd, J=5.6, 10.0 Hz, 1H), 3.36 (d, J=12 Hz, 1H), 3.28-3.16 (m, 1H), 2.37 (d, J=12 Hz, 1H), 2.26-2.10 (m, 2H), 1.95-1.82 (m, 1H), 1.79-1.75 (m, 2H), 1.66-1.59 (m, 2H), 1.28-1.10 (m, 6H), 1.07 (s, 9H), 1.00-0.87 (m, 10H), 0.61 (q, J=8.0 Hz, 6H), 0.50 (s, 3H).

Step 2. To a solution of compound 11-1 (15 g, 21.4 mmol, 1.0 eq.) in dried DCM (250 mL) cooled in an ice bath was added Dess-Martin reagent (11.8 g, 27.8 mmol, 1.3 eq.) in small portions. The reaction mixture was stirred at 30° C. for 2 h. TLC showed there was no starting material in the mixture. The mixture was poured into 100 mL of a saturated Na₂SO₃ aqueous solution and extracted with DCM (3×200 mL). The combined organic layers were washed with brine, dried over anhydrous Na₂SO₄ and concentrated to give the crude product, which was recrystallized using PE: EtOAc (10:1) to give compound 11-2 (11.5 g, 77% yield) as a white solid. ¹HNMR (400 MHz, CDCl₃) δ: 7.73-7.64 (m, 4H), 7.51-7.38 (m, 6H), 6.90-6.78 (m, 1H), 4.98 (d, J=4.8 Hz, 1H), 4.93 (s, 1H), 4.55 (s, 1H), 4.19 (m, 1H), 4.00 (dd, J=5.6, J=10.6 Hz, 1H), 3.80 (d, J=12Hz, 1H), 3.47 (d, J=12Hz, 1H), 2.52 (dt, J=5.8, J=14.8 Hz, 1H), 2.41 (d, J=12.8 Hz, 1H), 2.30-2.13 (m, 3H), 1.92 (m, 1H), 1.77-1.64 (m, 2H), 1.55-1.38 (m, 3H), 1.32-1.21 (m, 1H), 1.05 (m, 12H), 0.92 (t, J=8.0 Hz, 9H), 0.79 (s, 3H), 0.59-0.48 (q, J=8.0, J=16 Hz, 6H).

Step 3. To a solution of compound 11-2 (2.5 g, 3.57 mmol, 1.0 eq.) in dried THF (20 mL) was added HF/Pyr complex solution (15 mL) dropwise in an ice bath. The reaction mixture was stirred at 25° C. for 3.5 h. TLC (PE: EtOAc=1:3) showed there was about 20% of a partially deprotected compound in the mixture. The reaction mixture was poured into 100 mL of water and extracted with DCM (4×50 mL). The combined organic layers were washed with brine, dried with anhydrous Na₂SO₄ and concentrated to give the crude product, which was recrystallized from PE: EtOAc=5:1 to provide compound 11-3 (1.7 g, 68% yield) as a brown solid. ¹HNMR (400 MHz, CD₃OD) δ: 6.88 (t, J=4.0 Hz, 1H), 5.04 (d, J=4.0 Hz, 1H), 4.97 (s, 1H), 4.77 (s, 1H), 4.49 (dd, J=6.4, J=10.6 Hz, 1H), 4.18 (m, 1H), 4.04 (d, J=12.0 Hz, 1H), 3.50 (d, J=12.0 Hz Hz, 1H), 2.92-2.78 (m, 1H), 2.68 (t, J=6.8 Hz, 2H), 2.49 (d, J=12.0 Hz, 1H), 2.34 (td, J=4.0, J=16.0 Hz, 1H), 2.22-2.01 (m, 3H), 1.91-1.81 (m, 1H), 1.80-1.73 (m, 1H), 1.72-1.52 (m, 2H), 1.17 (s, 3H), 1.06 (s, 3H).

Step 4. A mixture of NH₂OH-HCl (0.32 g, 4.6 mmol, 1.6 eq.) and pyridine (2.3 mL, 28.7 mmol, 10 eq.) in anhydrous MeOH (8 mL) was added to a solution of compound 11-3 (1.0 g, 2.87 mmol, 1.0 eq.) in dry MeOH (7 mL) dropwise at room temperature. The mixture was stirred at room temperature for 10 min, and poured into 220 mL of water. 150 mL DCM was added to the mixture to dissolve the staring material. The solid was collected by filtration to give compound 11-4 (0.6 g, 58% yield) as a white solid. ¹HNMR (400 MHz, CDCl₃) δ: 8.60-8.50 (m, 1H), 7.50-7.41 (m, 1H), 6.87 ((t, J=4.0 Hz, 1H), 5.04 (d, J=8.0 Hz, 1H), 4.94 (s, 1H), 4.73 (s, 1H), 4.49 (dd, J=8.0, J=12.0 Hz, 1H), 4.18 (d, J=8.0 Hz, 1H), 3.87 (d, J=12.0 Hz, 1H), 3.43 (d, J=12.0 Hz, 1H), 3.26 (m, 1H), 2.70-2.59 (m, 2H), 2.48 (d, J=12.0 Hz, 1H), 2.20-2.02 (m, 2H), 2.01-1.87 (m, 3H), 1.66-1.57 (m, 1H), 1.55-1.44 (m, 1H), 1.42-1.31 (m, 1H), 1.24 (s, 3H), 0.91 (s, 3H).

Step 5. To a solution of compound 11-4 (0.2 g, 0.67 mmol, 1.0 eq.) in dry THF (10 mL) was added SOC1₂(0.2 g, 0.67 mmol, 1.0 eq.) in dry THF (10 mL) dropwise in an ice bath. The mixture was stirred for 5 min at 0° C. TLC showed there was trace staring material remained. A saturated NaHCO₃ (6 mL) aqueous solution was added, the ice bath was removed and the aqueous mixture was extracted with DCM: MeOH=15:1 (4×20 mL). The combined organic layers were washed with brine, dried over anhydrous Na₂SO₄ and concentrated to give the crude product, which was purified by preparative TLC using PE: EtOAc=1:3 to the titled compound with 95% purity. The crude product was recrystallized from PE: EtOAc=10:1 to give Ex.11 (0.1 g, 26% yield) as a white solid. Ex.11: ¹HNMR (400 MHz, CD₃OD) δ: 6.93-6.79 (t, J=8.0 Hz, 1H), 5.05 (d, J=4.0 Hz, 1H), 4.99 (s, 1H), 4.76 (s, 1H), 4.50 (dd, J=8.0, J=12.0 Hz, 1H), 4.19 (dd, J=4.0, J=12.0 Hz, 1H), 4.02 (q, J=8.0, J=20.0 Hz, 2H), 2.72-2.58 (m, 4H), 2.51-2.41 (m, 1H), 2.19-2.00 (m, 3H), 1.86-1.75 (m, 2H), 1.72 (d, J=12.0 Hz, 1H), 1.44 (dq, J=4.0, J=12.0 Hz, 1H), 1.27 (s, 3H), 0.78 (s, 3H); HPLC Purity: 97.4%; LCMS: M+H=346.2.

### Example 12

### Synthesis of (S,E)-3-(2-((3aR,5aS,6R,9aS,9bR)-5a,9b-dimethyl-7-methylene-3a,4,5,5a,6,7,8,9,9a,9b-decahydronaphtho[1,2-d]isoxazol-6-yl)ethylidene)-4-hydroxydihydrofuran-2(3H)-one

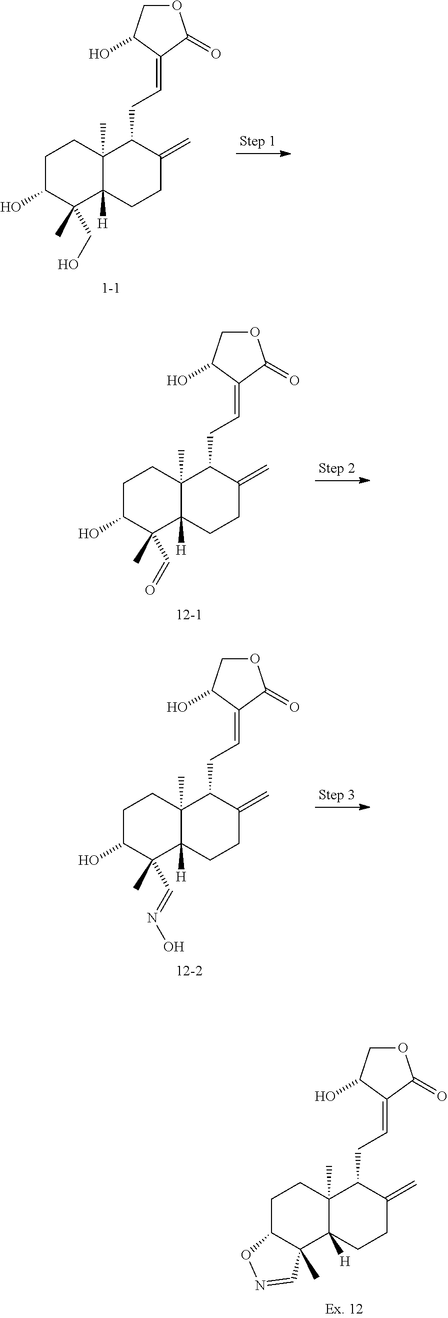 [SMILES: C=C1CC[C@@H]2[C@](C)(CO)[C@H](O)CC[C@@]2(C)[C@@H]1C/C=C1/C(=O)OC[C@H]1O ; C=C1CC[C@@H]2[C@](C)(C=O)[C@H](O)CC[C@@]2(C)[C@@H]1C/C=C1/C(=O)OC[C@H]1O ; C=C1CC[C@@H]2[C@](C)(/C=N/O)[C@H](O)CC[C@@]2(C)[C@@H]1C/C=C1/C(=O)OC[C@H]1O ; C=C1CC[C@@H]2[C@]3(C)C=NO[C@@H]3CC[C@@]2(C)[C@@H]1C/C=C1/C(=O)OC[C@H]1O]

Step 1. To a suspension of compound 1-1 (3.5 g, 10 mmol, 1.0 eq.) in dry dichloromethane (60 mL) cooled in an ice bath was added Dess-Martin reagent (2.1 g, 5 mmol) in small portions. The reaction mixture was stirred for 2 hours at 0° C. TLC indicated that about 50% of the starting material 1-1 still remained and two new spots were detected. A sat. Na₂SO₃ aqueous solution (100 mL) was added to the reaction mixture and stirring was continued for 10 min. The organic phase was separated and washed with brine. After concentration, the residue was purified by silica gel column chromatography (DCM: MeOH=100:1) to afford compound 12-1 (0.54 g, 17% yield) as a white solid. ¹H NMR (400 MHz, DMSO-d₆) δ: 9.97 (s, 1H), 6.69-6.57 (m, 1H), 5.82-5.71 (m, 1H), 5.19-5.15 (m, 1H), 4.96-4.91 (m, 1H), 4.86-4.82 (m, 1H), 4.67-4.63 (m, 1H), 4.44-4.38 (m, 1H), 4.09-4.02 (m, 1H), 3.37 (br. s., 1H), 2.62-2.42 (m, 3H), 2.32 (d, J=12.3 Hz, 1H), 1.97 (d, J=7.5 Hz, 3H), 1.90-1.74 (m, 3H), 1.45 (d, J=12.5 Hz, 1H), 1.39-1.29 (m, 1H), 1.18-1.05 (m, 4H), 0.57 (s, 3H).

Step 2. To a solution of compound 12-1 (348 mg, 1 mmol, 1.0 eq.) in dry CH₃OH (15 mL) was added a solution of NH₂OH.HCl (210 mg, 3 mmol, 3.0 eq.) and pyridine (1.2 g, 15 mmol, 15 eq.) in CH₃OH (5 mL) dropwise at 0° C.-5° C. The reaction mixture was stirred at 0° C.-5° C. for 10 min. TLC (EtOAc) indicated that the starting material 12-1 was consumed completely. The mixture was poured into water (150 mL), and extracted with EtOAc (30 mL×2). The combined organic phase was washed with brine and concentrated to afford compound 12-2 (350 mg, 98% yield) as a white solid, which was used directly in the next step. ¹HNMR (400 MHz, DMSO) δ: 10.39 (s, 1H), 8.62-8.53 (m, 1H), 7.84-7.75 (m, 1H), 7.44-7.35 (m, 3H), 6.68-6.60 (m, 1H), 5.74 (d, J=6.3 Hz, 1H), 4.93 (t, J=5.9 Hz, 1H), 4.84 (s, 1H), 4.74 (d, J=5.3 Hz, 1H), 4.63 (s, 1H), 4.45-4.37 (m, 1H), 4.08-4.01 (m, 2H), 3.20 (td, J=5.3, 10.7 Hz, 1H), 2.33 (d, J=13.1 Hz, 1H), 1.94 (d, J=10.5 Hz, 2H), 1.81-1.58 (m, 5H), 1.39-1.09 (m, 9H), 0.57 (s, 3H).

Step 3. To a solution of compound 12-2 (0.11 g, 0.3 mmol) in dry THF (4 mL) at 0° C. was added a solution of SOCl₂ (60 mg, 0.5 mmol, 1.7 eq) in THF (1 mL) dropwise. The resulting mixture was stirred for 2 min at 0° C. TLC indicated that compound 12-2 was consumed completely. A 10% NaHCO₃ aqueous solution (10 mL) was added to the mixture and stirred for 5 min. EtOAc (30×2 mL) was added to the mixture. The combined organic phases were washed with brine and concentrated. The residue was purified by pre-TLC (PE: EA=1:2) to afford the titled product Ex.12 (18.3 mg, 15%) as a white solid. Ex.12: ¹H NMR (400 MHz, Methanol-d₄) δ: 7.41 (s, 1H), 6.91-6.84 (m, 1H), 5.06-4.98 (m, 2H), 4.77 (s, 1H), 4.49 (dd, J=6.1, 10.2 Hz, 1H), 4.24-4.14 (m, 2H), 2.74-2.50 (m, 3H), 2.16 (dt, J=4.9, 12.9 Hz, 1H), 2.07-1.91 (m, 3H), 1.81 (td, J=3.5, 13.0 Hz, 1H), 1.71-1.64 (m, 1H), 1.60-1.20 (m, 4H), 1.17 (s, 3H), 0.76 (s, 3H); HPLC Purity: 99.2%; LCMS (ESI): M+H=346.2.

### Example 13

### Synthesis of (S,E)-4-hydroxy-3-(2-((3S,4aR,6aS,7R,10aS,10bR)-4a,6a,10b-trimethyl-8-methylene-3-oxidodecahydro-1H-naphtho[2,1-d][1,3,2]dioxathiin-7-yl)ethylidene)dihydrofuran-2(3H)-one

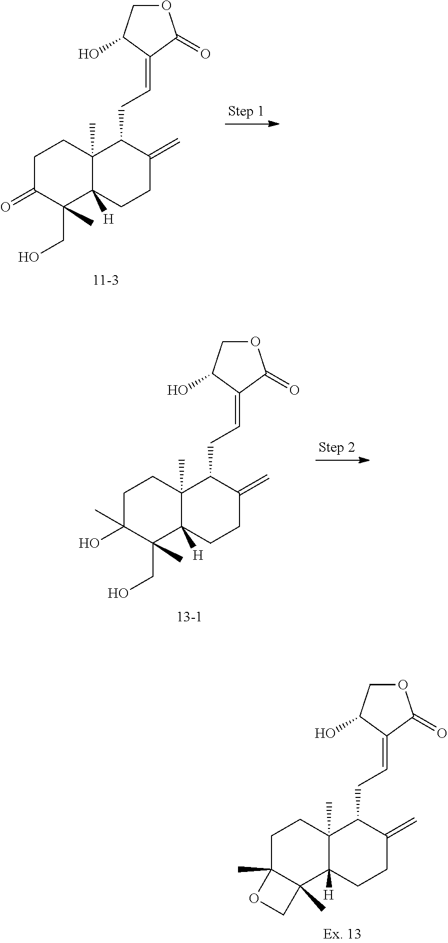 [SMILES: C=C1CC[C@H]2[C@@](C)(CCC(=O)[C@@]2(C)CO)[C@@H]1C/C=C1/C(=O)OC[C@H]1O ; C=C1CC[C@H]2[C@@](C)(CCC(C)(O)[C@@]2(C)CO)[C@@H]1C/C=C1/C(=O)OC[C@H]1O ; C=C1CC[C@H]2[C@@](C)(CC[C@@]3(C)OC[C@@]23C)[C@@H]1C/C=C1/C(=O)OC[C@H]1O]

Step 1. To a solution of compound 11-3 (250 mg, 1.0 mmol, 1.0 eq.), available from Preparative Example 11, in anhydrous THF (10.0 mL) was added MeLi (1M in THF, 3.6 mL, 3.6 mmol) dropwise at −78° C. under a N₂ atmosphere. The reaction mixture was stirred at −78° C. for 1.5 h. A saturated NH₄Cl aquesous solution was added to quench the reaction at −78° C. The two phases were separated, and the aqueous layer was extracted with EtOAc (5 mL×3). The combined organic extracts were dried over Na₂SO₄, filtered, and concentrated in vacuo to give a crude product, which was purified first by pre-TLC (PE: EA=1:1) and re-purified by prep. HPLC (Column: Phenomenex Gemini C18 200×25 mm×10 um, from 26% MeCN in water (0.05% FA) to 42% CH₃CN in Water (0.05% FA), Detection wavelength: 220 nm) to give compound 13-1 (35 mg, 52%) as a white solid. ¹HNMR (400 MHz, CDC13) δ: 7.00-6.96 (m, 1H), 5.05-5.03 (m, 1H), 4.91 (s, 1H), 4.60 (s, 1H), 4.49-4.45 (m, 1H), 4.28-4.22 (m ,1H), 3.33-3.10 (m, 2H), 2.56-2.42 (m, 2H), 2.16-1.75 (m, 5H), 1.55-1.30 (m, 4H), 1.29 (s, 3H), 1.29-1.11 (m, 1H), 1.10 (s, 3H), 0.71 (s, 3H).

Step 2. To a solution of compound 13-1 (500 mg, 1.37 mmol, 1.0 eq.) in THF (20 mL) was added SOCl₂ (326 mg, 2.74 mmol, 2.0 eq.) dropwise at 0° C. The reaction mixture was stirred at 0° C. for 0.5 h. TLC (PE/EA=1:1) showed the starting material was consumed. A saturated NaHCO₃ aqueous solution was added to quench the reaction. The two phases were separated, and the aqueous phase was extracted with EtOAc (20 mL×3). The combined organic phase was washed with brine, dried over Na₂SO₄, filtered, and concentrated to give a crude product, which was purified by prep. HPLC (Column: DIKMA Diamonsil C18 200×25×5 um, from 55% to 75% CH₃CN in water (0.1% FA)) to give Ex.13 (76.5 mg, 17.1%) as a white solid.

Ex.13: ¹HNMR (400 MHz, CDC13) δ: 6.96-6.93 (m, 1H), 5.29 (d, J=11.6H, 1H), 5.05 (br, s, 1H), 4.93 (s, 1H), 4.64 (s, 1H), 4.49-4.46 (m, 1H), 4.29-4.26 (m, 1H), 3.44-3.41 (m, 1H), 3.22-3.19 (m, 1H), 2.59-2.50 (m, 3H), 2.05-1.60 (m, 4H), 1.55-1.38 (m, 4H), 1.36 (s, 3H), 1.31 (s, 3H), 0.88 (s, 3H); HPLC Purity: 99.17%; HRMS: M+Na⁺=433.16.

### Example 14

### Synthesis of (S,E)-4-hydroxy-3-(2-((3aR,5aS,6R,9aS,9bR)-3a,5a,9b-trimethyl-7-methylene-3a,4,5,5a,6,7,8,9,9a,9b-decahydronaphtho[1,2-d]isoxazol-6-yl)ethylidene)dihydrofuran-2(3H)-one

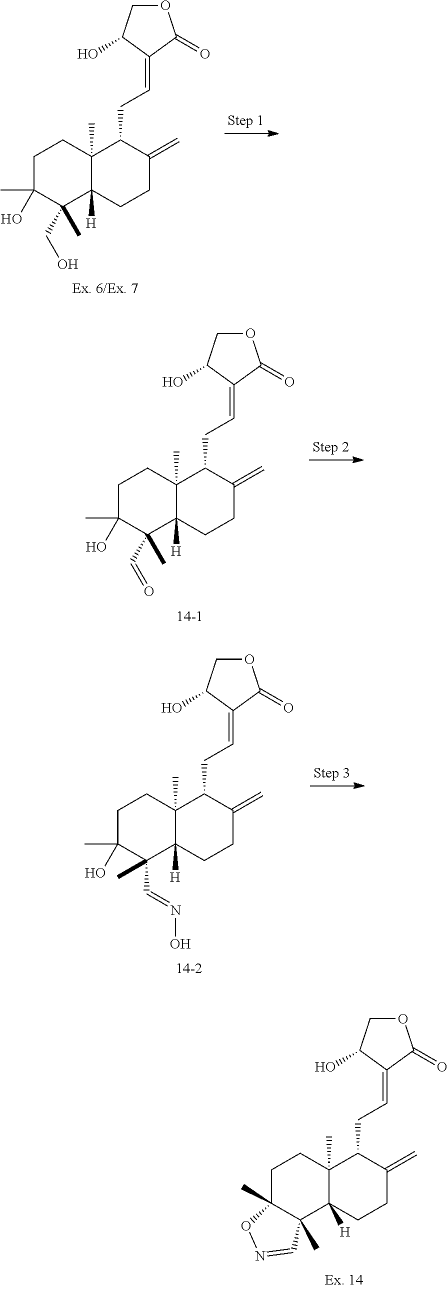

Step 1. To a mixture of Ex.6 and Ex.7 (300 mg, 0.824 mmol, 1.0 eq.), available from Preparative Examples 6 and 7, in dry DCM (30 mL) was added Dess-Martin reagent (367 mg, 0.865 mmol, 1.05 eq.) in small portions at 0° C. The mixture was stirred at 0° C. for 0.5 h. TLC (PE: EtOAc=1:1) showed the reaction was completed. A saturated aqueous Na₂SO₃ solution was added to quench the reaction. The two phases were separated, and the aqueous phase was extracted with DCM (20×3 mL). The combined organic phase was washed with brine, dried and concentrated to give the crude product, which was purified by column chromatography to give compound 14-1 (225 mg, 75%) as a white solid. ¹HNMR (400 MHz, CDCl₃) δ: 9.76 (s, 1H), 6.99-6.95 (m, 1H), 5.04 (br s, 1H), 4.96 (s, 1H), 4.64 (s, 1H), 4.51-4.46 (m, 1H), 4.29-4.26 (m, 1H), 2.59-2.57 (m, 3H), 2.05-1.59 (m, 7H), 1.22 (s, 3H), 1.17 (s, 3H), 0.89-0.86 (m, 2H), 0.71 (s, 3H).

Step 2. To a solution of compound 14-1 (30 mg, 0.08 mmol, 1.0 eq.) in MeOH (5 mL) was added pyridine (0.06 mL) and NH₂OH.HCl (8.9 mg, 0.13 mmol, 1.6 eq.) at 0° C. The reaction mixture was stirred at 0° C. for 10 min. Water was added, and the aqueous phase was extracted with EtOAc (5 mL×3). The combined organic phases were dried over Na₂SO₄, filtered, concentrated in vacuo to give a crude product, which was purified by prep. TLC (PE/EA=1:2) to give oxime 14-2 (4 mg, 12.9%). ¹HNMR (400 MHz, CDC13) δ: 7.46 (s, 1H), 7.00-6.98 (m, 1H), 5.05 (brs, 1H), 4.95 (s, 1H), 4.62 (s, 1H), 4.49-4.46 (m, 1H), 4.29-4.26 (m, 1H), 2.58-2.46 (m, 3H), 2.12-2.02 (m, 3H), 1.69-1.40 (m, 6H), 1.26 (s, 3H), 1.15 (s, 1H), 0.73 (s, 1H).

Step 3. To a solution of oxime 14-2 (135 mg, 0.36 mmol, 1.0 eq.) in THF (5.0 mL) was added SOCl₂ (85.7 mg, 0.72 mmol, 2.0 eq.) at 0° C. The reaction mixture was stirred at 0° C. for 5 min. A saturated NaHCO₃ aqueous solution was added to quench the reaction. The two phases were separated, and the aqueous phase was extracted with EtOAc (10 mL×3). The combined organic phase was washed with brine, dried over Na₂SO₄, filtered, and concentrated in vacuo to give a crude product, which was purified by prep. HPLC (Mobile phase A: water with 0.1% formic acid, Mobile phase B: acetonitrile; Column: DIKMA Diamonsil C18 200×25×5 um wavelength: 220 nm) to afford the titled compound Ex.14 (29.4 mg, 11.4%) as a white solid. Ex.14: ¹HNMR (400 MHz, CDCl₃) δ: 7.20 (s, 1H), 7.00-6.96 (m, 1H), 5.05 (brs, 1H), 4.99 (s, 1H), 4.67 (s, 1H), 4.50-4.46 (m, 1H), 4.29-4.26 (m, 1H), 2.59-2.54 (m, 2H), 1.95-1.65 (m, 8H), 1.43 (s, 3H), 1.37-1.12 (m, 2H), 0.99 (s, 3H), 0.80 (s, 3H); HPLC Purity: 99.3%; LCMS: M+H=360.0.

### Example 15

Synthesis of (4S,E)-3-(2-((1R,4aR,5R,8aR)-5,8a-dimethyl-6-(methylamino)-2-methylenedecahydronaphthalen-1-yl)ethylidene)-4-hydroxydihydrofuran-2(3H)-one

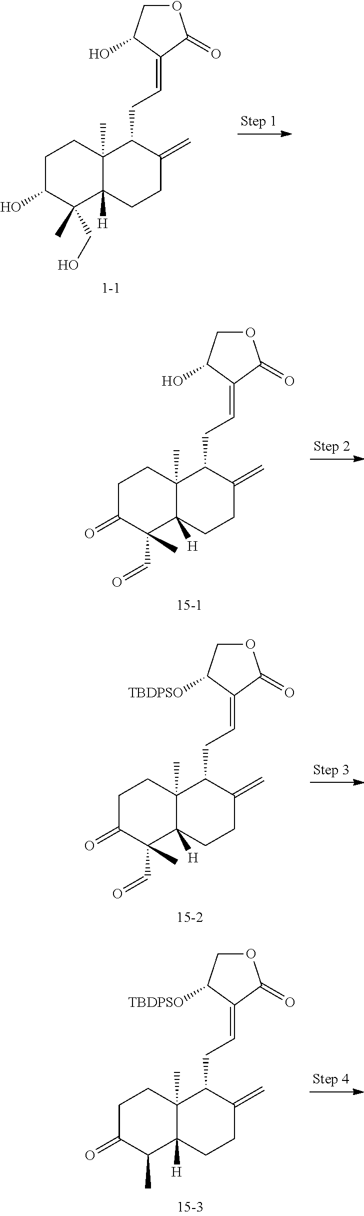

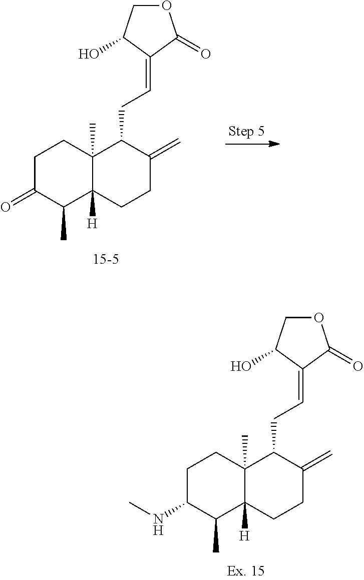 [SMILES: C=C1CC[C@@H]2[C@@H](C)C(=O)CC[C@@]2(C)[C@@H]1C/C=C1/C(=O)OC[C@H]1O ; C=C1CC[C@@H]2[C@@H](C)[C@H](NC)CC[C@@]2(C)[C@@H]1C/C=C1/C(=O)OC[C@H]1O]

Step 1. To a solution of compound 1-1 (100 g, 0.29 mol, 1.0 eq.) in DCM (1 L) cooled in an ice bath, was added Dess-Martin reagent (252 g, 0.59 mol, 2.05 eq) in small portions. The mixture was allowed to warm to r.t. and stirred for 18 h. TLC (PE: EA=1:1) indicated that compound 1-1 was consumed completely. The reaction mixture was poured into a cooled saturated aqueous Na₂SO₃ (2 L) solution and extracted with DCM (500 mL×3). The combined organic layers were washed with water (500 mL×3), brine (500 mL×3), dried over anhydrous sodium sulfate, and filtered. The filtrate was concentrated in vacuum, the residue was recrystallized from EA to afford compound 15-1 (100 g, crude) as a white solid. ¹H NMR (400 MHz, CDCl₃) δ: 9.70 (s, 1H), 6.95-6.92 (t, J=12 Hz, 1H), 5.04-5.02 (m, 2H), 4.72 (s, 1H), 4.50-4.46 (t, J=16 Hz ,1H), 4.27-4.24 (m, 1H), 2.70-2.43 (m, 6H), 2.18-1.90 (m, 4H), 1.86-1.79 (m, 1H), 1.62-1.61 (m, 2H), 1.55-1.50 (m, 2H), 1.29 (s, 3H), 0.93 (s, 3H).

Step 2. To a solution of compound 15-1 (11 g, 31.8 mmol, 1.0 eq.) in dry THF (160 mL) was added imidazole (21.6 g, 0.32 mol, 10.0 eq) followed by TBDPSCl (69.7 g, 0.25 mol, 8.0 eq) dropwise at 0-5° C. The resulting mixture was stirred for 3 h at r.t. Water (500 mL) was added to the mixture to quench the reaction, and the organic phase was separated and concentrated. The water layer was extracted with EA (100 mL×3); the combined organic layers were washed with water (100 mL×3), brine (100 mL×3), dried over anhydrous sodium sulfate and filtered. The filtrate was concentrated to a residue which was purified by silica gel column chromatography (PE: EtOAc=5: 1-3: 1) to afford compound 15-2 (6.6 g, 35.5%) as a light yellow gum. ¹HNMR (400 MHz, CDCl₃) (the spectra is from the pilot run batch) δ: 9.72 (s, 1H), 7.74-7.64 (m, 4H), 7.52-7.38 (m, 6H), 6.82 (t, J=6.1 Hz, 1H), 4.99 (s, 1H), 4.97 (s, 1H), 4.58 (s, 1H), 4.22 (dd, J=1.6, 10.2 Hz, 1H), 4.03 (dd, J=5.5, 10.2 Hz, 1H), 2.60-2.46 (m, 1H), 2.40-2.22 (m, 2H), 2.18-2.09 (m, 1H), 2.02-1.90 (m, 2H), 1.81-1.63 (m, 3H), 1.51 (td, J=3.2, 10.1 Hz, 1H), 1.32-1.23 (m, 5H), 1.04 (s, 9H), 0.47 (s, 3H).

Step 3. To a suspension of chloro(methoxymethyl)triphenylphosphorane (4.65 g, 13.6 mmol, 1.2 eq.) in THF (60 mL) at 0° C. was added LHMDS (13.6 mL, 1M in THF, 1.2 eq.) in portions. The mixture was stirred for 40 min at 0° C. Then a solution of compound 15-2 (6.6 g, 11.3 mmol, 1.0 eq.) in THF (100 mL) was added. The mixture was warmed to room temperature and stirred for 2 h, then poured into a saturated NH₄Cl aqueous solution (150 mL) slowly. The aqueous mixture was extracted with ethyl acetate (50 mL×3). The combined organic extracts were washed with brine (50 mL×3) and concentrated. The residue was purified by silica gel column chromatography (PE: EtOAc=5:1) to afford compound 15-3 (2.4 g, 38%) as a light yellow oil.

¹H NMR (400 MHz, CDCl₃) δ: 7.76-7.64 (m, 4H), 7.53-7.35 (m, 6H), 6.94-6.76 (m, 1H), 5.05-4.97 (m, 2H), 4.57 (s, 1H), 4.22-4.16 (m, 1H), 4.04-3.97 (m, 1H), 2.37-2.21 (m, 6H), 1.99-1.87 (m, 1H), 1.80-1.60 (m, 2H), 1.37-1.15 (m, 4H), 1.10-1.02 (m, 9H), 0.99-0.97 (d, J=6.4 Hz, 3H), 0.76 (s, 3H).

Step 4. A solution of compound 15-3 (1.4 g, 2.5 mmol, 1.0 eq.) in HF/Pyridine complex (14 mL) and THF (12 mL) was stirred for 3 h at 20° C. The mixture was poured into water (20 mL) and extracted with ethyl acetate (15 mL×3). The combined organic extracts were washed with water (20 mL×3), brine (20 mL×3), dried over anhydrous sodium sulfate and then concentrated. The residue was purified by silica gel column chromatography (PE: EtOAc=3: 1) to afford compound 15-4 (0.7 g, 88%) as a white solid. ¹H NMR (400 MHz, CDCl₃) δ: 6.98 (t, J=6.1 Hz, 1H), 5.05 (br. s., 1H), 5.00 (s, 1H), 4.69 (s, 1H), 4.48 (dd, J=6.1, 10.4 Hz, 1H), 4.27 (dd, J=1.6, 10.6 Hz, 1H), 2.66 (t, J=6.8 Hz, 2H), 2.53-2.29 (m, 5H), 2.15-1.94 (m, 3H), 1.84 (d, J=12.9 Hz, 1H), 1.58 (m, 1H), 1.45 (dt, J=2.9, 12.0 Hz, 1H), 1.36-1.22 (m, 1H), 1.01 (d, J=6.3 Hz, 3H), 0.98 (s, 3H).

Step 5. To a solution of compound 15-4 (500 mg, 1.57 mmol) in MeOH (16 mL) was added methylamine hydrochloride (530 mg, 7.85 mmol, 5.0 eq.) and pyridine (0.63 mL, 7.85 mmol, 5.0 eq.). The mixture was stirred for 10 min at 25° C. NaBH₃CN (970 mg, 15.7 mmol, 10.0 eq.) was added in one portion. Stirring was continued at 25° C. for 2 h. The reaction mixture was filtered, the filtrate was acidified by a 4N HCl aqueous solution to pH 2-3. The solution was concentrated at ambient temperature to about 10 mL and the residue was purified by pre-HPLC (Mobile phase A: water with 0.05% hydrochloric acid, Mobile phase B: acetonitrile; Column: Gemini 250×20 mm×5 um; Detection wavelength: 220 nm) to afford the titled product Ex.15 (38.1 mg, 7.3%) as a white solid. Ex.15: ¹H NMR (400 MHz, MeOD) δ: 6.84 (t, J=6.1 Hz, 1H), 5.01 (d, J=5.5 Hz, 1H), 4.94 (s, 1H), 4.72 (s, 1H), 4.46 (dd, J=6.1, 10.0 Hz, 1H), 4.15 (d, J=10.2 Hz, 1H), 2.88-2.78 (m, 1H), 2.72-2.61 (m, 6H), 2.43 (d, J=12.9 Hz, 1H), 2.14-1.88 (m, 5H), 1.71-1.55 (m, 2H), 1.41-1.10 (m, 3H), 1.02 (d, J=6.3 Hz, 3H), 0.77 (s, 3H); HPLC Purity: 93%; LCMS: M+H=333.9.

### Examples 16, 17, and 18

### Synthesis of (S,E)-4-hydroxy-3-(2-((1R,4aR,5R,6R,8aR)-6-methoxy-5,8a-dimethyl-2-methylenedecahydronaphthalen-1-yl)ethylidene)dihydrofuran-2(3H)-one (Ex.16)

### and (S,E)-4-hydroxy-3-(2-((1R,4aR,5R,6R,8aR)-6-methoxy-2,5,8a-trimethyl-1,4,4a,5,6,7,8,8a-octahydronaphthalen-1-yl)ethylidene)dihydrofuran-2(3H)-one (Ex.17)

### and (S,E)-4-hydroxy-3-(2-((4aR,5R,6R,8aR)-6-methoxy-2,5,8a-trimethyl-3,4,4a,5,6,7,8,8a-octahydronaphthalen-1-yl)ethylidene)dihydrofuran-2(3H)-one (Ex.18)

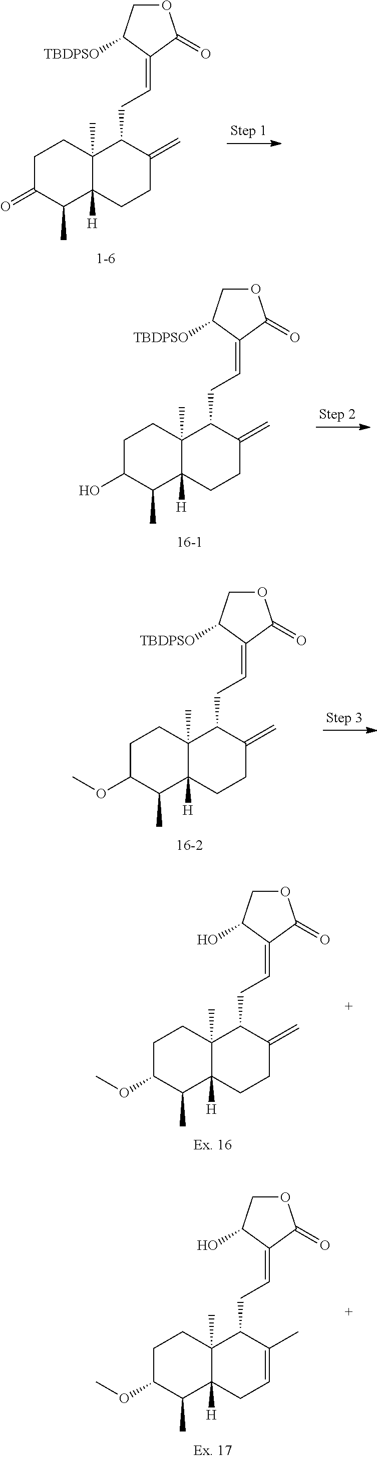

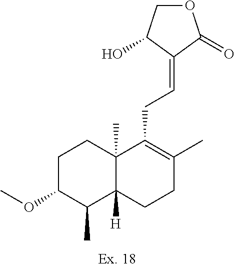

Step 1. To a suspension of compound 1-6 (2.0 g, 3.6 mmol, 1.0 eq.), available from Preparative Example 1, in MeOH (36 mL) was added HOAc (0.6 mL), NaBH₃CN (2.23 g, 36 mmol, 10.0 eq.) at ambient temperature. The mixture was stirred at 25° C. for 1.5 h. The reaction mixture was poured into water (100 mL) and extracted with ethyl acetate (60 mL×3). The combined organic phase was washed with water (20 mL×3), brine (20 mL×3), dried over anhydrous sodium sulfate and then concentrated. The residue was purified by silica column (PE: EtOAc=3:1) to afford the compound 16-1 (0.9 g, 45%) as a white solid. ¹H NMR (400 MHz, CDCl₃) δ: 7.76-7.64 (m, 4H), 7.53-7.35 (m, 6H), 6.94-6.76 (m, 1H), 5.05-4.99 (m, 1H), 4.89 (s, 1H), 4.50 (s, 1H), 4.22-4.16 (m, 1H), 4.04-3.97 (m, 1H), 3.09-3.04 (m, 1H), 2.36-2.26 (m, 3H), 1.99-1.85 (m, 1H), 1.80-1.60 (m, 3H), 1.45-1.15 (m, 3H), 1.10-1.02 (m, 10H), 0.99-0.97 (m, 5H), 0.53 (s, 3H).

Step 2. To a solution of compound 16-1 (280 mg, 0.5 mmol) in DCM (10 mL) was added 2,6-di-tert-butyl-4-methylpyridine methyl (1.03, 5 mmol, 10.0 eq.), trifluoromethanesulfonate (656 mg, 4 mmol, 8.0 eq.) at 25° C. via syringe. The mixture was stirred at 25° C. for 16 h, quenched with water (10 mL), and separated. The organic layer was washed with brine (50 mL×3), dried over anhydrous sodium sulfate and then concentrated. The residue was purified by silicon column chromatography (PE: EtOAc=8:1) to afford the compound 16-2 (0.12 g, 42%) as a white solid. ¹H NMR (400 MHz, CDCl₃) δ: 7.70 (d, J=6.7 Hz, 4H), 7.51-7.38 (m, 6H), 6.89 (t, J=6.1 Hz, 1H), 5.00 (br. s., 1H), 4.89 (s, 1H), 4.49 (s, 1H), 4.21-4.14 (m, 1H), 3.97 (dd, J=5.7, 10.0 Hz, 1H), 3.36 (s, 2.5H), 3.30 (s, 0.5H), 2.59 (dt, J=4.7, 10.6 Hz, 1H), 2.41-2.22 (m, 3H), 1.92 (d, J=12.1 Hz, 2H), 1.78 (d, J=11.0 Hz, 1H), 1.68 (d, J=9.4 Hz, 1H), 1.46-1.19 (m, 4H), 1.06 (s, 9H), 0.93 (d, J=6.3 Hz, 5H), 0.53 (s, 3H).

Step 3. To a solution of compound 16-2 (500 mg, 0.874 mmol) in THF (5 mL) was added a HF/Py complex. (8 mL) dropwise via syringe at 0-5° C. The mixture was stirred for 1 h at 25° C., poured into cold water (30 mL), and extracted with EA (20 mL×3). The combined organic layers were washed with brine (30 mL×3), dried over anhydrous sodium sulfate. After filtration and concentration, the residue was purified by pre-TLC (PE: EA=1:3) to afford a mixture of the three titled products (110 mg) as a colorless gum. The isomer mixture was separated by SFC to give Ex.16 (33.4 mg, 11.3%), Ex.17 (15.0 mg, 5.1%), Ex.18 (5.1 mg, 1.7%) as white solids.

Ex.16: ¹H NMR (400 MHz, CDCl₃) δ: 6.98 (br. s, 1H), 5.02 (s., 1H), 4.91 (s., 1H), 4.60 (s., 1H), 4.45 (s., 1H), 4.26 (d, J=9.8 Hz, 1H), 3.35 (s, 3H), 2.80 (s, 1H), 2.71-2.47 (m, 3H), 2.39 (d, J=12.1 Hz, 1H), 2.00 (m, 2H), 1.91-1.73 (m, 3H), 1.43-1.33 (m, 2H), 1.21-1.08 (m, 2H), 1.05-0.90 (m, 4H), 0.71 (br. s, 3H); NOE (400 MHz, CDCl₃): aided the stereoassignment; HPLC Purity: 99.6%; MS: M+Na=357.20.

Ex.17: ¹H NMR δ: 7.13 (t, J=6.7 Hz, 1H), 5.48 (br. s., 1H), 5.07 (br. s., 1H), 4.49 (dd, J=6.1, 10.4 Hz, 1H), 4.28 (d, J=10.6 Hz, 1H), 3.36 (s, 3H), 2.71-2.55 (m, 3H), 2.13 (d, J=17.6 Hz, 2H), 2.07-1.94 (m, 2H), 1.87 (d, J=12.9 Hz, 1H), 1.67 (br. s., 3H), 1.63-1.50 (m, 1H), 1.40-1.23 (m, 2H), 1.15-1.03 (m, 2H), 0.95 (d, J=6.3 Hz, 3H), 0.78 (s, 3H); NOE (400 MHz, CDCl₃): aided the stereoassignment; HPLC Purity: 100%; MS: M+Na=357.20.

Ex.18: ¹H NMR (400 MHz, CDCl₃) δ: 6.91-6.81 (m, 1H), 5.08 (br. s., 1H), 4.49 (dd, J=6.3, 10.2 Hz, 1H), 4.27 (dd, J=1.6, 10.6 Hz, 1H), 3.35 (s, 3H), 3.27 (dd, J=4.7, 17.2 Hz, 1H), 3.05 (dd, J=8.6, 17.2 Hz, 1H), 2.60 (m, 1H), 2.49 (br. s., 1H), 2.10-1.95 (m, 3H), 1.81-1.67 (m, 2H), 1.60 (s, 3H), 1.46-1.19 (m, 4H), 1.12 (m, 1H), 0.99-0.94 (m, 6H) ; NOE (400 MHz, CDCl₃) aided the stereoassignement; HPLC Purity: 100%; MS: M+H=335.22.

### Example 19

### Synthesis of N-((E)-4-(2-((4aR,6aS,7R,10aS,10bR)-6a,10b-dimethyl-8-methylene-3-oxidodecahydro-1H-naphtho[2,1-d][1,3,2]dioxathiin-7-yl)ethylidene)-5-oxotetrahydrofuran-3-yl)formamide

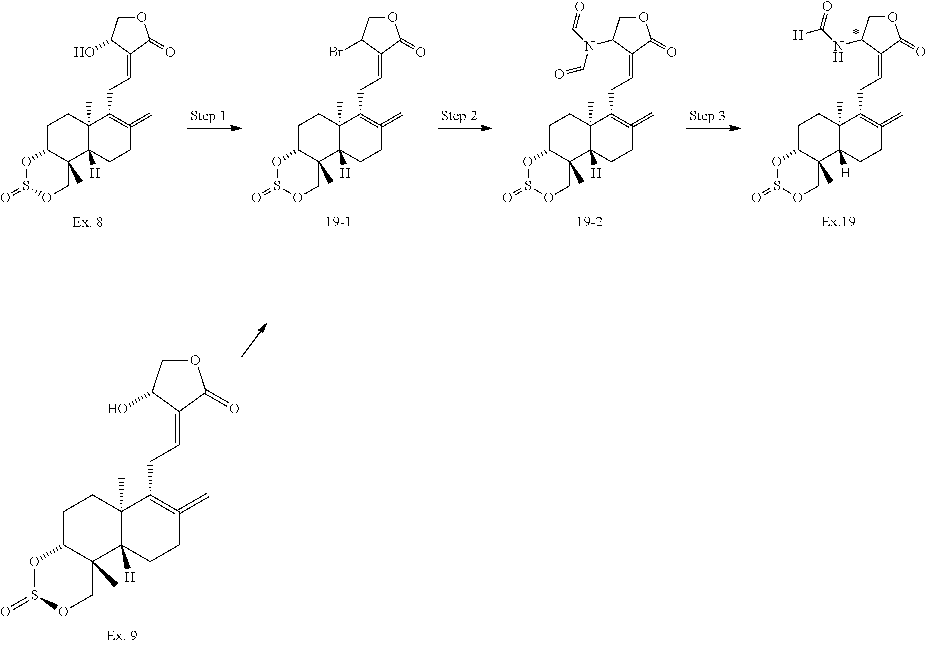

Step 1. To a mixture of compound Ex.8 (0.332 g, 1 mmol, 1.0 eq.), available from Preparative Example 8, and CBr₄ (0.664 g, 2 mmol, 2 eq) in DCM (10 mL) was added PPh₃ (0.524 g, 2 mmol, 2 eq.) in two portions. The reaction mixture was stirred at room temperature for 1 hour. TLC (PE: EA=1:1) showed some staring material Ex.8 was remained. The reaction mixture was concentrated and the residue was purified by column chromatography on silica gel eluted with PE: EtOAc=6:1 to give the bromide 19-1 (206 mg, 50%) as a white solid. Following the same procedure, compound Ex.9 was converted to bromide 19-1 similarily. ¹HNMR (400 MHz, CDCl₃) δ: 7.54-7.39 (m, 1H), 5.22-5.12 (m, 1H), 4.98 (d, J=12.0 Hz, 1H), 4.87 (d, J=8.0 Hz, 2H), 4.85-4.78 (m, 1H), 4.73 (d, J=16.8 Hz, 1H), 3.90-3.74 (m, 1H), 3.42 (t, J=12.3 Hz, 1H), 2.90-2.76 (m, 1H), 2.64 (t, J=12.4 Hz, 1H), 2.55-2.43 (m, 1H), 2.33-2.22 (m, 2H), 2.19-2.06 (m, 2H), 1.98-1.79 (m, 3H), 1.51-1.41 (m, 3H), 1.40-1.30 (m, 2H), 1.25-1.12 (m, 1H), 0.85 (s, 3H).

Step 2. A suspension of compound 19-1 (200 mg, 0.5 mmol, 1 eq) and sodium diformamide (60 mg, 0.6 mmol, 1.2 eq) in MeCN (4 mL) was stirred at room temperature overnight. The reaction mixture was directly purified by prep-TLC (PE: EA=1:1) to give the desired intermediate 19-2 (57 mg, 29%) as a white solid, which was used directly in the next step.

Step 3. A solution of compound 19-2 (39 mg, 0.1 mmol, 1 eq) in MeOH (3 mL) and MeCN (1 mL) was stirred at room temperature overnight. The solvent was removed under reduced pressure and the residue was purified by prep-HPLC (Mobile phase A: water with 0.1% formic acid, Mobile phase B: acetonitrile; Column: Agela DuraShell 200×25 mm×5 um; Detection wavelength: 220 nm) to give the titled product Ex.19 (17 mg, 47%), a diasteromeric mixture, as a white solid. Ex.19: ¹HNMR: 17883-102-1 (400 MHz, CDCl₃) δ: 8.28 (d, J=3.8 Hz, 1H), 7.04-6.86 (m, 1H), 6.03 (br. s., 1H), 5.40 (br. s., 1H), 5.17 (d, J=11.3 Hz, 1H), 4.94 (d, J=15.1 Hz, 1H), 4.59 (td, J=6.9, 10.3 Hz, 1H), 4.52-4.41 (m, 1H), 4.27-4.18 (m, 1H), 3.83 (dd, J=4.5, 13.1 Hz, 1H), 3.43 (d, J=11.3 Hz, 1H), 2.92-2.76 (m, 1H), 2.56-2.32 (m, 3H), 2.30-2.19 (m, 1H), 2.08-1.96 (m, 1H), 1.92-1.76 (m, 3H), 1.46 (s, 3H), 1.41-1.27 (m, 2H), 1.07 (t, J=13.6 Hz, 1H), 0.87 (d, J=2.5 Hz, 3H); HPLC Purity: 97.02%; LCMS: M+H=360.0.

### Examples 20 and 21

### N-((3S,E)-4-(2-(((4aR,6aS,7R,10aS,10bR)-6a,10b-dimethyl-8-methylene-3-oxidodecahydro-1H-naphtho[2,1-d][1,3 ,2]dioxathiin-7-yl)ethylidene)-5-oxotetrahydrofuran-3-yl)formamide (Ex.20)

### and N-((3R,E)-4-(2-((4aR,6aS,7R,10aS,10bR)-6a,10b-dimethyl-8-methylene-3-oxidodecahydro-1H-naphtho[2,1-d][1,3,2]dioxathiin-7-yl)ethylidene)-5-oxotetrahydrofuran-3-yl)formamide (Ex.21)

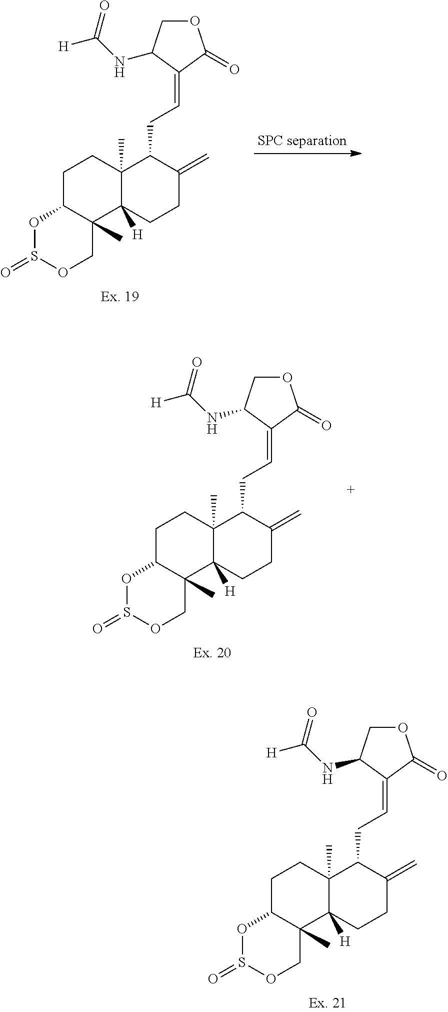

A diastereomeric mixture Ex.19 (160 mg) was separated by SFC separation (neutral) to provide the titled pure diastereomers Ex. 20 (32.2 mg, 20%) and Ex. 21 (30.3 mg, 19%) as white solids. The stereoconfiguration of the amide group is tentatively assigned.

Ex.20: ¹HNMR (400 MHz, CHLOROFORM-d) δ: 8.24 (s, 1H), 6.98-6.85 (m, 1H), 6.43 (d, J=7.5 Hz, 1H), 5.37 (br. s., 1H), 5.14 (d, J=11.3 Hz, 1H), 4.92 (s, 1H), 4.61-4.51 (m, 1H), 4.48 (s, 1H), 4.20 (d, J=8.8 Hz, 1H), 3.80 (dd, J=4.3, 12.8 Hz, 1H), 3.40 (d, J=11.3 Hz, 1H), 2.88-2.70 (m, 1H), 2.47 (d, J=12.8 Hz, 2H), 2.40-2.30 (m, 1H), 2.28-2.17 (m, 1H), 2.07-1.95 (m, 1H), 1.82 (t, J=10.8 Hz, 3H), 1.43 (s, 3H), 1.37-1.25 (m, 2H), 1.10-0.99 (m, 1H), 0.83 (s, 3H); HPLC Purity: 100%; LCMS: [M+Na]=446.0.

Ex.21: ¹HNMR (400 MHz, CHLOROFORM-d) δ: 8.25 (br. s., 1H), 6.86 (br. s., 1H), 6.11 (br. s., 1H), 5.39 (br. s., 1H), 5.15 (d, J=11.3 Hz, 1H), 4.90 (br. s., 1H), 4.64-4.51 (m, 1H), 4.41 (br. s., 1H), 4.21 (d, J=9.8 Hz, 1H), 3.80 (d, J=9.3 Hz, 1H), 3.41 (d, J=11.0 Hz, 1H), 2.90-2.72 (m, 1H), 2.54-2.30 (m, 3H), 2.22 (d, J=11.5 Hz, 1H), 2.01 (d, J=11.5 Hz, 1H), 1.91-1.74 (m, 3H), 1.44 (s, 3H), 1.37-1.21 (m, 2H), 1.04 (t, J=12.5 Hz, 1H), 0.85 (s, 3H); HPLC Purity: 98.65%; LCMS: [M+Na]=446.0.

### Example 22

### Synthesis of N-((E)-5-oxo-4-(2-((4aR,6aS,7R,10aS,10bR)-4a,6a,10b-trimethyl-8-methylene-3-oxidodecahydro-1H-naphtho[2,1-d][1,3,2]dioxathiin-7-yl)ethylidene)tetrahydrofuran-3-yl)formamide

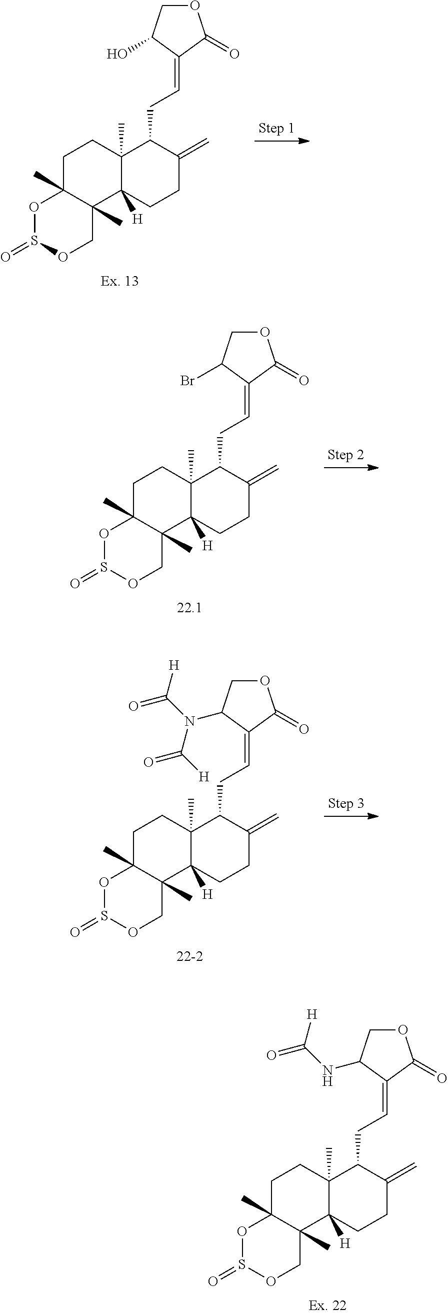 [SMILES: C=C1CC[C@H]2[C@@](C)(CC[C@@]3(C)O[S@](=O)OC[C@@]23C)[C@@H]1C/C=C1/C(=O)OC[C@H]1O ; C=C1CC[C@H]2[C@@](C)(CC[C@@]3(C)OS(=O)OC[C@@]23C)[C@@H]1C/C=C1/C(=O)OCC1Br ; C=C1CC[C@H]2[C@@](C)(CC[C@@]3(C)OS(=O)OC[C@@]23C)[C@@H]1C/C=C1/C(=O)OCC1N(C=O)C=O ; C=C1CC[C@H]2[C@@](C)(CC[C@@]3(C)OS(=O)OC[C@@]23C)[C@@H]1C/C=C1/C(=O)OCC1NC=O]

Step 1. To a mixture of compound Ex.13 (200 mg, 0.58 mmol, 1.0 eq.), available from Preparative Example 13, and CBr₄ (385 mg, 1.16 mmol, 2.0 eq) in DCM (10 mL) was added PPh₃ (304 mg, 1.16 mmol, 2.0 eq.) in two portions. The reaction mixture was stirred at room temperature for 2 hour. TLC (PE: EA=1: 1) showed the staring material still remained. The reaction mixture was concentrated and the residue was purified by column chromatography on silica gel eluted with PE: EtOAc (6:1) to give the desired product 22-1 (41 mg, 17.3%) as a solid.

¹HNMR (400 MHz, CDCl₃) δ: 7.54-7.51 (m, 1H), 5.29-5.26 (m, 1H), 4.86-4.68 (m, 4H), 3.76-3.73 (m, 1H), 3.43-340 (m, 1H), 3.18-3.13 (m, 1H), 2.49-2.46 (m, 1H), 2.02-1.26 (m, 16H), 0.79 (s, 3H).

Step 2. A suspension of compound 22-1 (200 mg, 0.49 mmol, 1 eq) and sodium diformamide (95 mg, 1.0 mmol, 2.0 eq) in MeCN (30 mL) was stirred at room temperature overnight. The solvent was removed under reduced pressure and the residue was purified by prep.TLC (PE: EA=1:1) to give the desired product 22-2 (125 mg, 63.8%) as a solid. ¹HNMR (400 MHz, CDCl₃) δ: 8.83 (br s, 2H), 6.79-6.65 (m, 1H), 5.74 (s, 1H), 5.20-5.17 (m, 1H), 4.83-4.80 (m, 1H), 4.54 (m, 1H), 4.29-4.05 (m, 2H), 3.35-3.32 (m, 1H), 3.09-3.06 (m, 1H), 2.36 (m, 1H), 2.24-1.17 (m, 16H), 0.76 (m, 3H).

Step 3. A solution of compound 22-2 (250 mg, 0.62 mmol, 1 eq) in MeOH (50 mL) was stirred at room temperature overnight. The solvent was removed under reduced pressure and the residue was purified by prep. HPLC (Mobile phase A: water with 0.1% formic acid, Mobile phase B: acetonitrile; Column: Boston C18 150×30 mm×5 um; Detection wavelength: 220 nm) to give the titled product Ex.22 (69.5 mg, 29.9%) as a white solid. Ex.22: ¹HNMR (400 MHz, CDCl₃) δ: 8.29-8.26 (m, 1H), 7.00-6.89 (m, 1H), 5.95-5.90 (m, 1H), 5.39 (br s, 1H), 5.29-5.26 (m, 1H), 4.93-4.89 (m, 1H), 4.60-4.58 (m, 1H), 4.59-4.41 (m, 1H), 4.24-4.20 (m, 1H), 3.43-3.40 (m, 1H), 3.25-3.15 (m, 1H), 2.46-2.44 (m, 3H), 2.06-2.00 (m, 2H), 1.95-1.55 (m, 4H), 1.52-1.38 (m, 2H), 1.38 (s, 3H), 1.31 (s, 3H), 0.85 (s, 3H); HPLC Purity: 98.23%; LCMS: M+H=438.1.

### Example 23

### Synthesis of (Z)-4-hydroxy-3-(2-((1′S,4a′S,8a′S)-5′,5′,8a′-trimethyloctahydro-1′H-spiro[cyclopropane-1,2′-naphthalen]-1′-yl)ethylidene)dihydrofuran-2(3H)-one

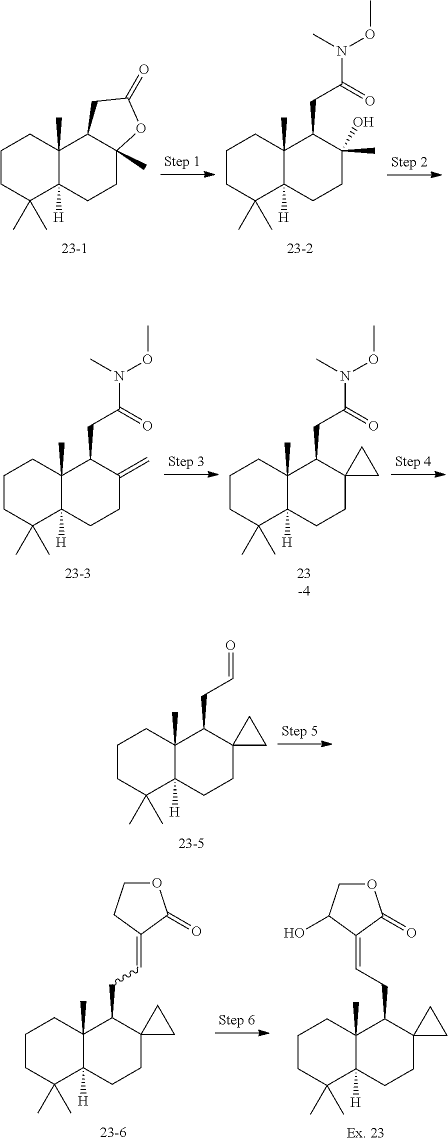

Step 1. To a suspension of MeNHOMe.HCl (70.2 g, 720 mmol, 3.0 eq.) in DCM (500 mL) was added AlMe₃ (360 mL, 2 M in toluene, 240 mmol, 3.0 eq.) dropwise under N₂ at −70° C. The reaction mixture was stirred at room temperature for 3 hours. A solution of commercially available compound 23-1 (60 g, 240 mmol, 1.0 eq.) in DCM (100 mL) was added dropwise to the reaction mixture at 0° C. The reaction mixture was stirred at room temperature for 10 hours and quenched with aq. NH₄Cl solution (500 mL) at 0° C. The reaction mixture was diluted with DCM (800 mL), the insoluble material was removed by filtration, and the aqueous layer was extracted with DCM (500 mL×3). The combined organic layers were washed with water (300 mL×3), brine (300 mL×3), dried over anhydrous sodium sulfate, filtered and concentrated. The residue was purified by column (PE: EA=5: 1˜1: 3) to give compound 23-2 (42 g, 56%) as a light yellow solid. ¹HNMR (400 MHz, CDCl₃) δ: 3.72 (s, 3H), 3.19 (s, 3H), 2.50-2.39 (m, 1H), 2.01-1.91 (m, 2H), 1.85 (br. s., 1H), 1.69-1.13 (m, 10H), 1.03 (dd, J=1.9, 12.2 Hz, 1H), 0.87 (s, 3H), 0.83 (s, 3H), 0.79 (s, 3H).

Step 2. To a solution of compound 23-2 (42 g, 135 mmol, 1.0 eq.) in DCM (500 mL) was added pyridine (106.6 g, 1350 mmol, 10.0 eq.) and SOC1₂ (49 mL, 675 mmol, 5.0 eq.) under N₂ at −70° C. The reaction mixture was stirred at room temperature for 1 h. The reaction was quenched with aq. NH₄Cl solution (200 mL) at 0° C. After separation, the water layer was extracted with DCM (200 mL×3). The combined organic layer was washed with water (300 mL×3), brine (200 mL×3), dried over anhydrous sodium sulfate, filtered and concentrated. The residue was purified by column chromatography on silica gel (petroleum ether: EtOAc=10:1) to give compound 23-3 (8 g, 20%) as a white solid. ¹H NMR (400 MHz, CDCl₃) δ: 4.74 (s, 1H), 4.45 (s, 1H), 3.73 (s, 3H), 3.17 (s, 3H), 2.77-2.64 (m, 1H), 2.51 (d, J=10.5 Hz, 1H), 2.44-2.33 (m, 2H), 2.15 (dt, J=4.8, 12.7 Hz, 1H), 1.74 (m, 1H), 1.64-1.14 (m, 8H), 0.89 (s, 3H), 0.82 (s, 3H), 0.74 (s, 3H).

Step 3. To a stirred solution of compound 23-3 (8 g, 27.3 mmol, 1.0 eq.) in DCM (250 mL) was added Et₂Zn (1 M in THF, 218.4 mL, 218.4 mmol, 8.0 eq.) at 0° C. The mixture was stirred at ambient temperature for 40 min and then added with CH₂ICl (48 g, 273 mmol, 10 eq.) via an additional funnel, the temperature was maintained below 25° C. using cold water. The stirring was continued for another 1 h, and the reaction was quenched by addition of water (100 mL) carefully. After separation, the water layer was extracted with DCM (50 mL×3). The combined organic layer was washed with water (100 mL×3), brine (100 mL×3), dried over anhydrous sodium sulfate. The organic phase was filtered and concentrated to give crude product 23-4 (6 g, 71.5%) as a white solid. ¹HNMR (400 MHz, CDCl₃) δ: 3.64 (s, 3H), 3.14 (s, 3H), 2.31 (dd, J=3.5, 7.0 Hz, 1H), 2.17 (dd, J=3.4, 17.4 Hz, 1H), 1.90 (m, 1H), 1.66-1.33 (m, 8H), 1.18-1.01 (m, 3H), 0.88 (s, 3H), 0.86 (s, 3H), 0.83 (s, 3H), 0.47-0.38 (m, 1H), 0.19 (td, J=4.9, 9.5 Hz, 1H), 0.12-0.05 (m, 1H), −0.07 (td, J=5.4, 9.2 Hz, 1H).

Step 4. To a solution of compound 23-4 (6 g, 19.5 mmol, 1.0 eq.) in THF(100 mL) was added DIBAL (1 M in toluene, 39 mL, 39 mmol, 2.0 eq.) dropwise under N₂ at −78° C. The reaction mixture was stirred at −78° C. for 1 h, and quenched with aq. NH₄Cl solution (150 mL). The mixture was filtered, and the water layer was extracted with DCM (100 mL×3). The combine organic layer was washed with water (100 mL×3), brine (100 mL×3), dried over anhydrous sodium sulfate, filtered and concentrated. The residue was purified by column chromatography on silica gel (petroleum ether: EtOAc=80: 1) to give the aldehyde 23-5 (3.1 g, 64%) as a white solid. ¹H NMR (400 MHz, CDCl₃) δ: 9.67 (s, 1H), 2.28-2.16 (m, 2H), 1.88 (m, 1H), 1.65-1.36 (m, 8H), 1.25-1.14 (m, 1H), 1.10-1.03 (m, 1H), 0.99-0.92 (m, 2H), 0.88 (m, 6H), 0.85 (s, 3H), 0.47-0.38 (m, 1H), 0.30-0.21 (m, 1H), 0.14-0.06 (m, 1H), −0.03 (m, 1H).

Step 5. To a mixture of LHMDS (1M in THF, 14.2 mL, 14.2mmol, 1.3 eq.) was added 2-dihydrofuranone (1.12 g, 13.1 mmol, 1.2 eq.) in THF (3 mL) dropwise at −70° C. The mixture was stirred for 1 hour then a solution of the aldehyde 23-5 (2.7 g, 10.9 mmol, 1.0 eq.) in THF (10 mL) was added via addition funnel. The stirring was continued for another 1 h. TLC indicated the aldehyde 23-5 was consumed completely. TEA (3.0 mL, 21.8 mmol, 2.0 eq.) was added followed by MsCl (2.32 g, 20.4 mmol, 1.8 eq.) at 0° C. The reaction mixture was warmed to ambient temperature and stirred for 2 h. The resulting suspension was diluted with dry THF (20 mL), and DBU (2.57 g, 16.4 mmol 1.5 eq.) was added dropwise. The yellow suspension was stirred at ambient temperature for 12 h, quenched by a sat. NH₄Cl aqueous solution (50 mL). The reaction mixture was extracted with EA (50 mL×3). The combined organic layer was washed with water (100 mL×3), brine (100 mL×3), dried over anhydrous sodium sulfate, filtered and concentrated. The residue was purified by column chromatography on silica gel (PE: EA=20: 1) to give compound 23-6 (2.57 g, 74.7%) as a yellow solid.

¹HNMR (400 MHz, CDCl₃) Isomer 1: δ: 6.77-6.65 (m, 1H), 4.42-4.32 (m, 2H), 2.85-2.76 (m, 2H), 2.10-1.99 (m, 1H), 1.86-1.75 (m, 1H), 1.66-1.59 (m, 3H), 1.57 (d, J=2.5 Hz, 1H), 1.50-1.36 (m, 2H), 1.29 (td, J=7.0, 16.4 Hz, 1H), 1.17 (dt, J=3.9, 13.4 Hz, 1H), 1.01-0.90 (m, 3H), 0.88 (s, 6H), 0.84 (s, 3H), 0.53-0.44 (m, 1H), 0.24 (m, 2H), 0.03-−0.08 (m, 1H).

Isomer 2: δ: 6.21-6.10 (m, 1H), 4.37-4.25 (m, 2H), 2.89 (dt, J=2.4, 7.3 Hz, 2H), 2.04-1.90 (m, 1H), 1.84-1.69 (m, 2H), 1.66-1.52 (m, 3H), 1.50-1.33 (m, 4H), 1.15 (dt, J=3.9, 13.5 Hz, 1H), 0.99-0.93 (m, 2H), 0.93-0.86 (m, 6H), 0.84 (s, 3H), 0.63-0.56 (m, 1H), 0.30 (td, J=4.7, 9.3 Hz, 1H), 0.22 (td, J=4.9, 9.3 Hz, 1H), −0.10 (td, J=5.0, 9.3 Hz, 1H).

Step 6. To a solution of compound 23-6 (0.3 g, 0.95 mmol, 1.0 eq.) in dioxane (5 mL) was added SeO₂ (105 mg, 0.95 mmol, 1.0 eq.) in one portion. The mixture was heated to 60° C. for 18 h. Upon cooling, the solvent was removed under reduced pressure and the residue was purified by column chromatography on silica gel eluted with PE: EA=10:1˜3:1 to give a mixture of isomers (50 mg, 4.0%). Further purification by prep-HPLC (Mobile phase A: water with 0.075% TFA, Mobile phase B: acetonitrile; Column: Synergi Max-RP 150×30 mm×4 um; Detection wavelength: 220 nm) provided the titled product Ex.23 (5.7 mg, 0.45%) as a white solid. Only trace of the E-analog of the titled product was detected, not isolated. Ex.23: ¹HNMR (400 MHz, CDCl₃) δ: 6.55 (t, J=6.7 Hz, 1H), 4.83 (d, J=3.3 Hz, 1H), 4.42 (dd, J=6.0, 10.0 Hz, 1H), 4.16 (dd, J=2.8, 10.0 Hz, 1H), 2.63 (d, J=17.8 Hz, 1H), 2.06 (td, J=7.2, 17.9 Hz, 1H), 1.87-1.69 (m, 2H), 1.63-1.52 (m, 3H), 1.47-1.38 (m, 3H), 1.16 (dt, J=4.0, 13.6 Hz, 1H), 1.01-0.94 (m, 2H), 0.93-0.87 (m, 8H), 0.85 (s, 3H), 0.58 (d, J=6.3 Hz, 1H), 0.30-0.20 (m, 2H), -0.04-0.13 (m, 1H); NOE (400 MHz, CDCl₃): aided the structural assignment; HPLC purity: 99.7%; LCMS: M+H: 333.0.

### Example 24

Synthesis of N—((S,E)-5-oxo-4-(2-((1′S,4a′S,8a′S)-5′,5′,8a′-trimethyloctahydro-1′H-spiro[cyclopropane-1,2′-naphthalen]-1′-yl)ethylidene)tetrahydrofuran-3-yl)formamide

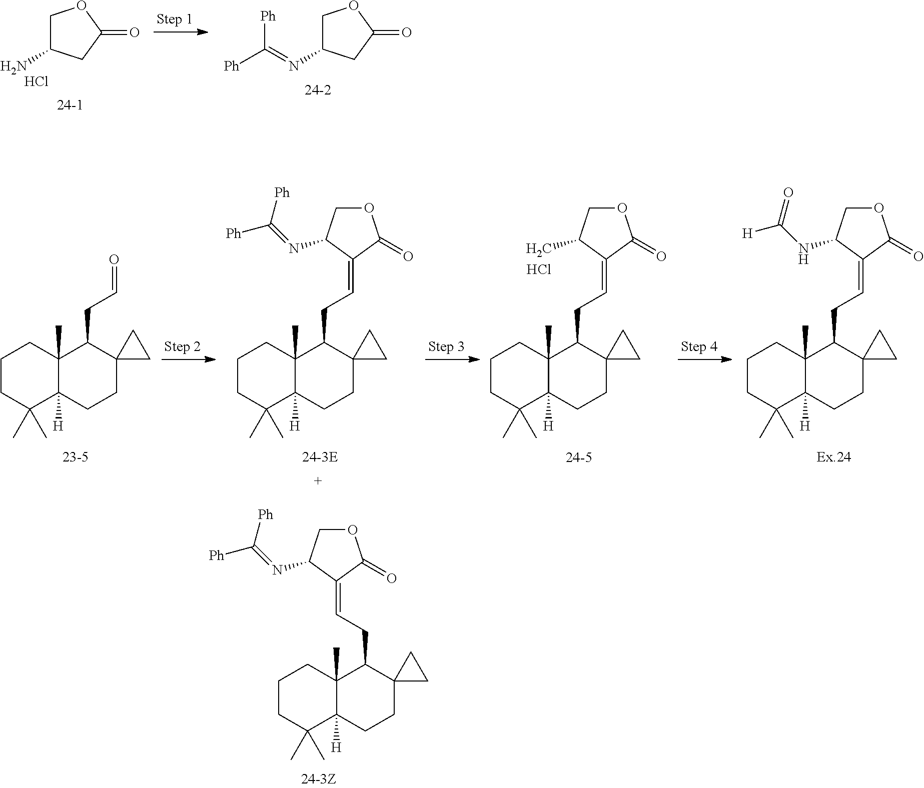

Step 1. Commercially available 3S-amino-2-furanone 24-1 was converted to the benzophenone imine analog 24-2 using a common literature procedure.

Step 2. To a stirred solution of LHMDS (1 M in THF, 14.4 mL, 14.4 mmol, 1.2 eq.) in THF (25 mL) was added imine compound 24-2 (3.82 g, 14.4 mmol, 1.2 eq.) in THF (20 mL) drop wise at −70° C. The mixture was stirred for 1 h, then a solution of aldehyde 23-5 (3 g, 12 mmol, 1 eq.), prepared in Preparative Example 23, in THF (30 mL) was added slowly via an addition funnel. Stirring was continued for another 1 h and reaction progress was monitored by TLC until the aldehyde was consumed completely. TEA (3.3 mL, 24 mmol, 2 eq.) and MsCl (2.1 g, 18 mmol, 1.5 eq.) was added at 0° C. The mixture was warmed to ambient temperature and stirred for 1 h. The resulting suspension was diluted with dry THF (10 mL) and cooled to 0° C. DBU (2.74 g, 18 mmol, 1.5 eq.) was added dropwise. The yellow suspension was stirred at ambient temperature for 2 h and quenched by a saturated NH₄Cl aqueous solution (30 mL). The two layers were separated, the aqueous layer was extracted with EA (30 mL×3), and the combined organic layer was washed with water (30 mL×3), brine (30 mL×3), dried over anhydrous sodium sulfate. Removal of solvents afforded a residue which was purified by column chromatography on silica gel (PE: EA=15:1) to give compound 24-3E (2.5 g, 42%) and its isomer compound 24-3Z (1.0 g, 16.8%) as colorless oils.

24-3E: ¹HNMR (400 MHz, CDCl₃) δ: 7.66 (d, J=7.0 Hz, 2H), 7.54-7.48 (m, 3H), 7.48-7.41 (m, 1H), 7.40-7.34 (m, 2H), 7.19-7.12 (m, 2H), 5.98 (dt, J=1.9, 6.8 Hz, 1H), 4.71-4.62 (m, 1H), 4.33-4.20 (m, 2H), 2.75-2.63 (m, 1H), 2.00-1.89 (m, 1H), 1.85-1.75 (m, 1H), 1.68 (d, J=12.5 Hz, 1H), 1.58 (d, J=3.5 Hz, 1H), 1.51-1.46 (m, 1H), 1.46-1.33 (m, 4H), 1.27 (br. s., 1H), 1.19-1.08 (m, 1H), 0.99-0.94 (m, 2H), 0.87 (m, 6H), 0.84 (s, 3H), 0.63-0.53 (m, 1H), 0.42 (m, 1H), 0.27-0.17 (m, 1H), -0.08 (m, 1H).

24-3Z: ¹HNMR (400 MHz, CDCl₃) δ: 7.61 (d, J=7.3 Hz, 2H), 7.56-7.49 (m, 3H), 7.47-7.40 (m, 1H), 7.39-7.31 (m, 2H), 7.16-7.09 (m, 2H), 6.87-6.79 (m, 1H), 4.69 (d, J=5.5 Hz, 1H), 4.37 (t, J=8.3 Hz, 1H), 4.24-4.10 (m, 1H), 2.16-2.04 (m, 1H), 1.76 (m, 1H), 1.66-1.58 (m, 2H), 1.58-1.51 (m, 1H), 1.50-1.43 (m, 1H), 1.43-1.28 (m, 4H), 1.20-1.05 (m, 2H), 0.99-0.88 (m, 2H), 0.86 (s, 3H), 0.84-0.81 (m, 1H), 0.79 (s, 3H), 0.75 (s, 3H), 0.17-0.05 (m, 2H), 0.00-−0.09 (m, 1H), −0.19 (m, 1H).

Step 3. To a solution of compound 24-3E (2.5 g, 5 mmol, 1.0 eq.) in THF (50 mL) was added a 2 N HCl aqueous solution (5 mL). The mixture was stirred at ambient temperature for 2 h. The mixture was directly dried over anhydrous sodium sulfate, filtered and concentrated to give a crude product as a white solid. PE (20 mL) was added and the suspension was stirred at ambient temperature for 30 min, filtered and the filter cake was collected to provide the HCl salt of amino compound 24-4 (1.9 g, >100%) as a white solid. 30 mg of this material of 24-4 was recrystallized in DCM: MeOH (10:1) to give a pure product 24-4 (15.1 mg, 50%).

¹HNMR (400 MHz, DMSO-d6) δ: 8.53 (br. s., 0.2H), 6.84 (d, J=8.5 Hz, 1H), 4.64 (d, J=6.0 Hz, 1H), 4.54 (dd, J=7.0, 10.5 Hz, 1H), 4.32 (d, J=10.5 Hz, 1H), 2.42 (d, J=18.6 Hz, 1H), 1.84-1.62 (m, 4H), 1.61-1.48 (m, 2H), 1.47-1.31 (m, 3H), 1.24-1.09 (m, 1H), 1.06-0.94 (m, 2H), 0.88 (d, J=8.0 Hz, 7H), 0.83 (s, 3H), 0.64 (br. s., 1H), 0.25-0.15 (m, 1H), 0.13-0.03 (m, 1H), −0.09-−0.18 (m, 1H); HPLC purity: 100%; LCMS: M+H=332.0.

Step 4. To a suspension of compound 24-4 (734 mg, 2.0 mmol, 1.0 eq.) in THF(10 mL) was added formic anhydride (freshly prepared, 2.6 mmol, 1.3 eq.) dropwise. The mixture was stirred at 25° C. for 18 h, then quenched with a NaHCO₃ aqueous solution. The mixture was separated, and the organic layer was washed with brine (20 mL×3), dried over anhydrous sodium sulfate, filtered and concentrated. The residue was purified by column chromatography on silica gel (PE: EA=1:1) to give the titled product Ex.24 (211.6 mg, 29.5%) as a white solid. Ex.24: ¹HNMR (400 MHz, CDCl₃) δ: 8.23 (s, 1H), 6.94 (br. s., 1H), 5.29 (t, J=6.7 Hz, 1H), 4.55 (dd, J=7.2, 10.2 Hz, 1H), 4.20 (d, J=10.3 Hz, 1H), 2.19 (dd, J=2.5, 17.6 Hz, 1H), 1.80 (t, J=12.0 Hz, 1H), 1.68-1.54 (m, 5H), 1.50-1.35 (m, 4H), 1.24-1.13 (m, 1H), 1.02-0.91 (m, 3H), 0.88 (d, J=5.3 Hz, 6H), 0.84 (s, 3H), 0.58-0.50 (m, 1H), 0.30-0.23 (m, 1H), 0.17-0.10 (m, 1H), 0.01-0.07 (m, 1H); HPLC purity:100%; LCMS: M+H=360.1.

### Example 25

Synthesis of N—((S,Z)-5-oxo-4-(2-((1′S,4a′S,8a′S)-5′,5′,8a′-trimethyloctahydro-1′H-spiro[cyclopropane-1,2′-naphthalen]-1′-yl)ethylidene)tetrahydrofuran-3-yl)formamide

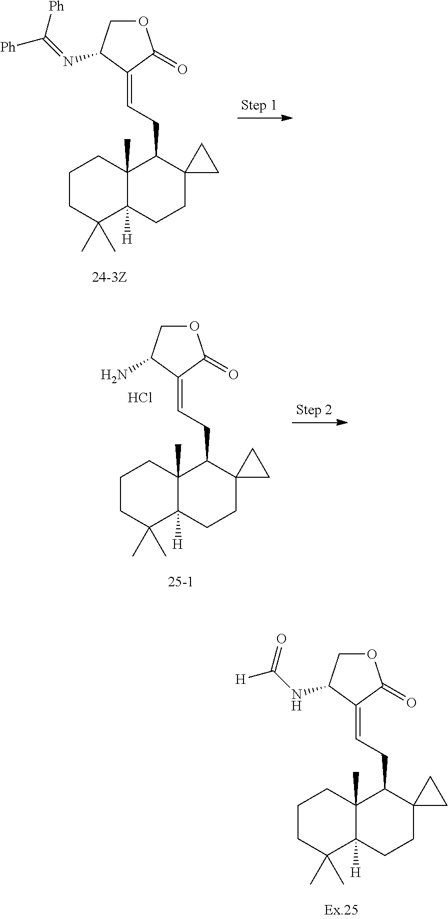 [SMILES: CC1(C)CCC[C@@]2(C)[C@H]1CCC1(CC1)[C@@H]2C/C=C1\C(=O)OC[C@H]1N=C(c1ccccc1)c1ccccc1 ; CC1(C)CCC[C@@]2(C)[C@H]1CCC1(CC1)[C@@H]2C/C=C1\C(=O)OC[C@H]1N ; Cl ; CC1(C)CCC[C@@]2(C)[C@H]1CCC1(CC1)[C@@H]2C/C=C1\C(=O)OC[C@H]1NC=O]

Step 1. To a solution of compound 24-3Z (0.8 g, 1.6 mmol, 1.0 eq.), prepared in Example 24, in THF (8 mL) was added a 2 N HCl aqueous solution (1.6 mL). The mixture was stirred at ambient temperature for 2 h. The mixture was dried over sodium sulfate, filtered, and concentrated to give the crude product as a white solid. PE (10 mL) was added to the residue and the suspension was stirred at ambient temperature for 30 min, filtered, and the filter cake was collected to give the desired product 25-1 (0.38 g, 63.6%) as a white solid. ¹HNMR (400 MHz, DMSO-d6) δ: 8.41 (br. s., 2H), 6.82 (t, J=7.0 Hz, 1H), 4.57-4.48 (m, 2H), 4.19 (d, J=8.0 Hz, 1H), 1.97-1.84 (m, 1H), 1.75 (t, J=11.8 Hz, 1H), 1.65 (d, J=12.5 Hz, 1H), 1.59-1.47 (m, 3H), 1.38 (d, J=11.5 Hz, 3H), 1.19-1.07 (m, 1H), 1.00-0.90 (m, 3H), 0.86 (br. s., 6H), 0.83 (s, 3H), 0.48 (m., 1H), 0.43-0.33 (m, 1H), 0.28-0.20 (m, 1H), −0.03-−0.13 (m, 1H).

Step 2. To a suspension of compound 25-1 (274 mg, 0.74 mmol, 1.0 eq.) in THF (10 mL) was added formic anhydride (1.48 mmol, 2.0 eq.) dropwise. The mixture was stirred at 25° C. for 18 h and quenched with a NaHCO₃ aqueous solution. The two phases were separated, the organic layer was washed with brine (15 mL×3), dried over anhydrous sodium sulfate, filtered and concentrated. The resulting residue was purified by p-TLC (PE: EA=1:3) to give the titled product Ex.25 (58.5 mg, 21.2%) as a white solid. Ex.25: ¹HNMR (400 MHz, CDCl₃) δ: 8.26 (s, 1H), 6.49 (t, J=6.5 Hz, 1H), 6.02 (br. s., 1H), 5.22 (br. s., 1H), 4.61-4.52 (m, 1H), 4.13-4.04 (m, 1H), 2.67 (m, 1H), 2.02-1.90 (m, 1H), 1.80 (t, J=12.3 Hz, 1H), 1.67 (d, J=12.1 Hz, 1H), 1.58 (d, J=13.7 Hz, 2H), 1.54-1.47 (m, 1H), 1.46-1.37 (m, 3H), 1.15 (m, 1H), 1.00-0.93 (m, 2H), 0.89 (d, J=11.0 Hz, 7H), 0.84 (s, 3H), 0.56 (d, J=3.5 Hz, 1H), 0.22 (m, 2H), −0.04-−0.12 (m, 1H); HPLC purity: 98.9%; LCMS: M+H=360.1.

### Example 26

### Synthesis of N—((S,E)-5-oxo-4-(2-((1′S,4a′S,8a′S)-5′,5′,8a′-trimethyloctahydro-1′H-spiro[cyclopropane-1,2′-naphthalen]-1′-yl)ethylidene)tetrahydrofuran-3-yl)acetamide

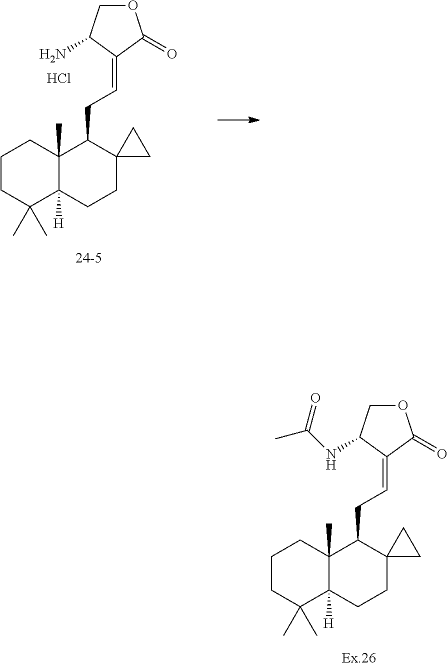 [SMILES: CC1(C)CCC[C@@]2(C)[C@H]1CCC1(CC1)[C@@H]2C/C=C1/C(=O)OC[C@H]1N ; Cl ; CC(=O)N[C@@H]1COC(=O)/C1=C/C[C@H]1C2(CC[C@H]3C(C)(C)CCC[C@@]31C)CC2]

To a suspension of intermediate 24-5 (0.44 g, 1.2 mmol, 1.0 eq.), available from Preparative Example 24, and TEA (0.5 mL 3.6 mmol, 3.0 eq.) in DCM (12 mL) was added AcCl (141.3 mg, 1.8 mmol, 1.5 eq.) dropwise at 0° C. The mixture was stirred at ambient temperature for 30 min. and quenched by water (20 mL). The two phases were separated; the organic layer was washed with water (15 mL×3), brine (15 mL×3), dried over anhydrous sodium sulfate, filtered and concentrated to give a crude product as a light yellow solid. This crude product was recrystallized from PE: DCM (10:1) to provide the titled product Ex.26 (146.9 mg, 32.8%) as a white solid. Ex.26: ¹HNMR (400 MHz, CDCl₃) δ: 6.92 (t, J=6.3 Hz, 1H), 6.01 (br. s., 1H), 5.25-5.14 (m, 1H), 4.51 (dd, J=7.0, 10.0 Hz, 1H), 4.19 (dd, J=1.8, 10.3 Hz, 1H), 2.19 (dd, J=4.8, 16.8 Hz, 1H), 2.02 (s, 3H), 1.87-1.75 (m, 1H), 1.61 (d, J=14.1 Hz, 4H), 1.50-1.39 (m, 4H), 1.24-1.12 (m, 1H), 0.96 (dd, J=13.1, 16.6 Hz, 3H), 0.88 (d, J=4.0 Hz, 6H), 0.85 (s, 3H), 0.53 (d, J=3.5 Hz, 1H), 0.27 (m 1H), 0.17 (m, 1H), −0.02 (m, 1H); HPLC purity: 100%; LCMS: M+H=374.3.

### Example 27

### Synthesis of N—((S,Z)-5-oxo-4-(2-((1′S,4a′S,8a′S)-5′,5′,8a′-trimethyloctahydro-1′H-spiro[cyclopropane-1,2′-naphthalen]-1′-yl)ethylidene)tetrahydrofuran-3-yl)acetamide

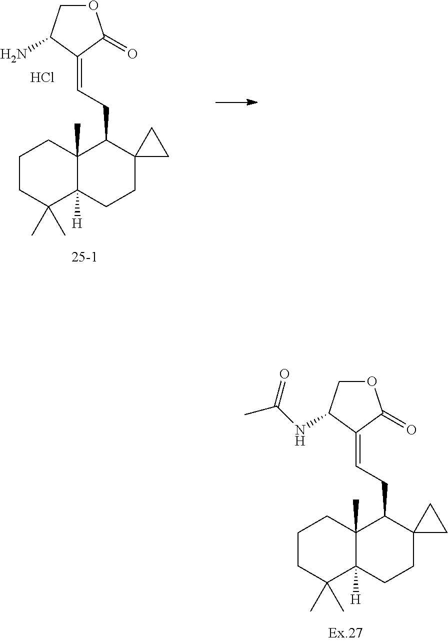 [SMILES: CC1(C)CCC[C@@]2(C)[C@H]1CCC1(CC1)[C@@H]2C/C=C1\C(=O)OC[C@H]1N ; Cl ; CC(=O)N[C@@H]1COC(=O)/C1=C\C[C@H]1C2(CC[C@H]3C(C)(C)CCC[C@@]31C)CC2]

To a suspension of intermediate 25-1 (0.2 g, 0.54 mmol, 1.0 eq.), available from Preparative Example 25, and TEA (0.25 mL 1.62 mmol, 3.0 eq.) in DCM (5 mL) was added AcCl (64 mg, 0.81 mmol, 1.5 eq.) dropwise under ambient temperature. The mixture was stirred for 30 min, quenched by water (10 mL) and separated. The organic layer was washed with water (10 mL×3), brine (10 mL×3), dried over anhydrous sodium sulfate, filtered, and concentrated to give a crude product as a light yellow solid which was purified by p-TLC (PE: EA=1:3) to afford the titled product Ex.27 (64.3 mg, 32.0%) as a white solid. Ex.27: ¹HNMR: 17780-29-1 (400 MHz, CDCl₃) δ: 6.46 (t, J=6.5 Hz, 1H), 5.80 (br. s., 1H), 5.10 (d, J=2.0 Hz, 1H), 4.53 (m, 1H), 4.06 (m, 1H), 2.70 (d, J=16.0 Hz, 1H), 2.05 (s, 3H), 1.97-1.86 (m, 1H), 1.85-1.75 (m, 1H), 1.70-1.63 (m, 1H), 1.61-1.48 (m, 4H), 1.47-1.36 (m, 3H), 1.15 (m, 1H), 1.00-0.93 (m, 2H), 0.90 (s, 3H), 0.87 (s, 3H), 0.84 (s, 3H), 0.56 (d, J=4.3 Hz, 1H), 0.29-0.18 (m, 2H), 0.03-0.11 (m, 1H); HPLC purity: 99.64%; LCMS: M+H=374.0.

### Examples 28 and 29

### Synthesis of N-((3S,Z)-4-(2-((4aR,6aS,7R,10aS,10bR)-6a,10b-dimethyl-8-methylene-3-oxidodecahydro-1H-naphtho[2,1-d][1,3,2]dioxathiin-7-yl)ethylidene)-5-oxotetrahydrofuran-3-yl)formamide (Ex.28) and

### N-((3R,Z)-4-(2-((4aR,6aS,7R,10aS,10bR)-6a,10b-dimethyl-8-methylene-3-oxidodecahydro-1H-naphtho [2,1-d][1,3,2]dioxathiin-7-yl)ethylidene)-5-oxotetrahydrofuran-3-yl)formamide (Ex.29)

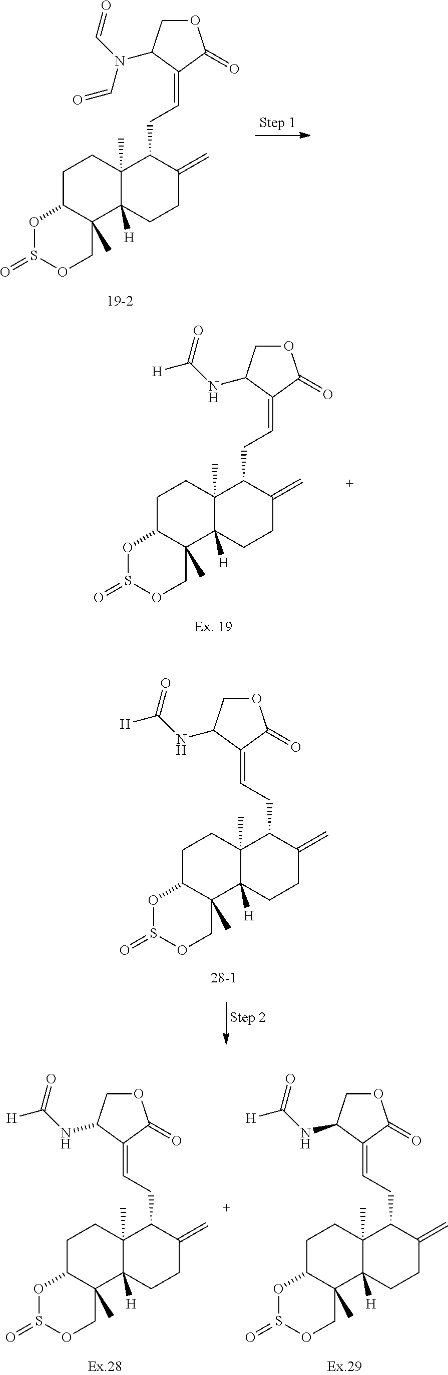

Step 1. A solution of compound 19-2 (2 g, 5.17 mmol, 1.0 eq) in MeOH (200 mL) and MeCN (40 mL) was stirred at room temperature for 3 days. Based on TLC (PE: EA=1:1) assessment of the reaction mixture, the starting material 19-2 was largely remained. The reaction mixture was heated to 50° C. for 2 h. Solvent was removed under reduced pressure and the residue was purified by prep-HPLC (Mobile phase A: water with 0.1% formic acid, Mobile phase B: acetonitrile; Column: Agela DuraShell 200×25 mm×5 um; Detection wavelength: 220 nm) to give the E-isomer Ex.19 (0.62 g, 33%) and the Z-isomer 28-1 (0.14 g, 7.5%) as white solids. The geometry of the alkene bond was assigned based on noe data.

Compound Ex.19 was separated by SFC separation (neutral) to give Ex.20 (330 mg, 55%) and Ex.21(140 mg, 23%) both as white solids. Detailed spectroscopic data is included in Example 20 and Example 21.

Step 2. The diastereomer mixture 28-1 (0.14 g) was separated by SFC separation (neutral) to give the pure isomers Ex.28 (45 mg, 32%) and Ex.29 (60 mg, 43%) both as white solids. The stereoconfiguration for the amido group is tentatively assigned.

Ex.28: ¹HNMR (400 MHz, CHLOROFORM-d) δ: 8.20 (s, 1H), 6.49-6.40 (m, 1H), 6.26 (d, J=7.3 Hz, 1H), 5.19 (br. s., 1H), 5.17-5.09 (m, 1H), 4.87 (s, 1H), 4.59-4.48 (m, 2H), 4.07 (dd, J=4.0, 10.0 Hz, 1H), 3.85-3.75 (m, 1H), 3.40 (d, J=11.5 Hz, 1H), 2.96-2.70 (m, 3H), 2.51-2.37 (m, 1H), 2.27-2.12 (m, 1H), 2.05-1.69 (m, 4H), 1.42 (s, 3H), 1.38-1.19 (m, 2H), 1.08 (dt, J=2.8, 13.6 Hz, 1H), 0.90-0.81 (m, 3H). HPLC Purity: 98.2%. SFC Purity: 100%. LCMS: [M+Na]⁺=446.0.

Ex.29: ¹HNMR (400 MHz, CHLOROFORM-d) δ: 8.18 (s, 1H), 6.51-6.36 (m, 2H), 5.23-5.08 (m, 2H), 4.86 (s, 1H), 4.58-4.47 (m, 2H), 4.12-4.00 (m, 1H), 3.80 (dd, J=4.3, 12.8 Hz, 1H), 3.39 (d, J=11.5 Hz, 1H), 2.96 (dd, J=6.5, 16.1 Hz, 1H), 2.86-2.65 (m, 2H), 2.43 (d, J=13.6 Hz, 1H), 2.27-1.71 (m, 4H), 1.41 (s, 3H), 1.36-1.18 (m, 3H), 1.16-1.04 (m, 1H), 0.84 (s, 3H). HPLC Purity: 99.25%. SFC: Purity: 100%. LCMS: [M+Na]⁺=446.0.

### Examples 30 and 31

### Synthesis of methyl ((3S,E)-4-(2-((4aR,6aS,7R,10aS,10bR)-6a,10b-dimethyl-8-methylene-3-oxidodecahydro-1H-naphtho[2,1-d][1,3,2]dioxathiin-7-yl)ethylidene)-5-oxotetrahydrofuran-3-yl)carbamate (Ex.30) and methyl ((3R,E)-4-(2-((4aR,6aS,7R, 10aS,10bR)-6a,10b-dimethyl-8-methylene-3-oxidodecahydro-1H-naphtho[2,1-d][1,3,2]dioxathiin-7-yl)ethylidene)-5-oxotetrahydrofuran-3-yl)carbamate (Ex.31)

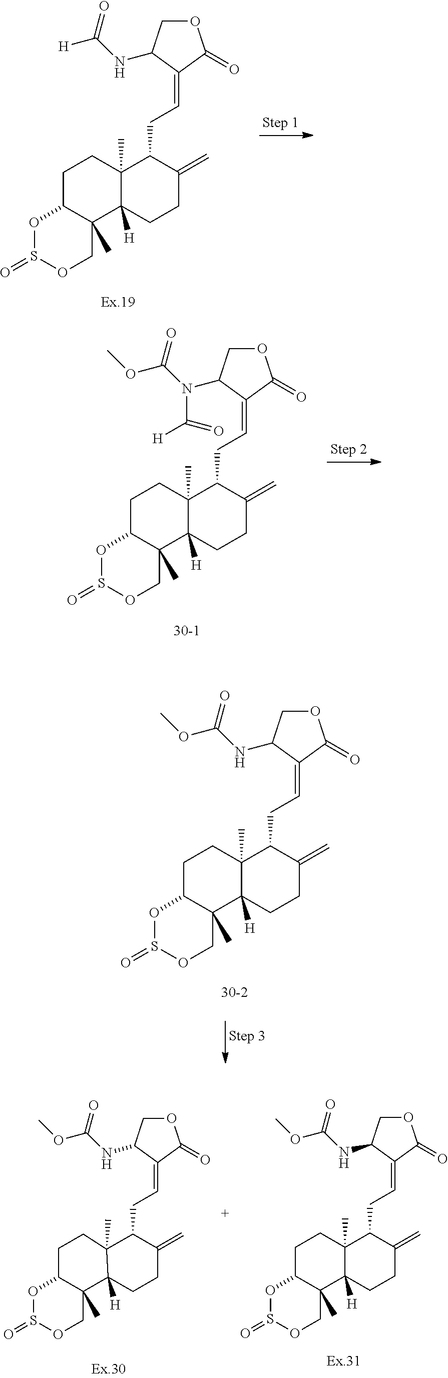

Step 1. A solution of LHMDS (1M in THF, 1.8 mL, 1.8 mmol, 1.5 eq) in 10 mL of anhydrous THF was added compound Ex.19 (430 mg, 1.2 mmol, 1 eq, available from Example 19) in 1 mL of THF dropwise at −78° C. under N₂ atmosphere. The resulting mixture was stirred at −78° C. for 0.5 h. Methyl chloroformate (340.5 mg, 3.6 mmol, 3 eq) in THF (2 mL) was added. The reaction mixture was stirred for additional 1 h. TLC (PE/EA=1:2) indicated the completion of reaction. The mixture was quenched by a saturated NH₄Cl aqueous solution (10 mL), extracted with EtOAc (20 mL×3), dried over Na₂SO₄, and concentrated in vacuum to give the crude product of compound 30-1 (600 mg, gum) which was used directly in the next step without further purification.

Step 2. To a solution of compound 30-1 (600 mg crude, 1.2 mmol, 1 eq) in MeOH (10 mL) and MeCN (5 mL) was added NaHCO₃ (151 mg, 1.8 mmol, 1.5 eq). The mixture was stirred at 25° C. for 5 h. LCMS indicated the completion of the reaction. The reaction mixture was concentrated, the residue was diluted with EtOAc (50 mL) and washed with water (10 mL×2). The organic phase was concentrated and the residue (500 mg crude) was purified by prep-HPLC (Mobile phase A: water with 0.1% formic acid, Mobile phase B: acetonitrile; Column: Agela DuraShell 200×25 mm×5 um; Detection wavelength: 220 nm) to give the carbamate product 30-2 (146 mg, 31%) as a white solid.

Step 3. The diastereomeric mixture 30-2 (146 mg) was separated by SFC (Mobile phase: Superaritical CO₂/EtOH; Column: Chiralpak AD (250×30 mm×5 um; Detection wavelength: 220 nm) to afford pure isomers Ex.30 (37 mg) and Ex.31 (68 mg) as white solids. The stereoconfiguration of the amino groups are arbitrary assigned.

Ex.30: ¹HNMR (400 MHz, CHLOROFORM-d) δ: 6.93 (t, J=6.0 Hz, 1H), 5.18-5.09 (m, 2H), 5.05 (br. s., 1H), 4.92 (br. s., 1H), 4.57-4.44 (m, 2H), 4.24-4.16 (m, 1H), 3.80 (dd, J=4.2, 12.8 Hz, 1H), 3.71 (s, 3H), 3.40 (d, J=11.3 Hz, 1H), 2.89-2.72 (m, 1H), 2.54-2.29 (m, 3H), 2.23 (dd, J=3.4, 14.0 Hz, 1H), 1.99 (t, J=12.6 Hz, 1H), 1.89-1.74 (m, 3H), 1.42 (s, 3H), 1.36-1.23 (m, 2H), 1.05 (t, J=12.5 Hz, 1H), 0.83 (s, 3H). HPLC Purity: 100%. SFC: de=100%.

LCMS: M+H: 454.

Ex.31: ¹HNMR (400 MHz, CHLOROFORM-d) δ: 6.98-6.79 (m, 1H), 5.14 (d, J=11.5 Hz, 1H), 5.07 (br. s., 2H), 4.89 (br. s., 1H), 4.53 (d, J=6.1 Hz, 1H), 4.44 (br. s., 1H), 4.20 (d, J=9.8 Hz, 1H), 3.86-3.76 (m, 1H), 3.76-3.62 (m, 3H), 3.40 (d, J=11.3 Hz, 1H), 2.90-2.71 (m, 1H), 2.56-2.28 (m, 3H), 2.20 (d, J=11.0 Hz, 1H), 1.98 (t, J=12.6 Hz, 1H), 1.89-1.74 (m, 3H), 1.43 (s, 3H), 1.36-1.23 (m, 2H), 1.03 (t, J=12.7 Hz, 1H), 0.84 (s, 3H). HPLC Purity: 97%.

SFC: de=100%. LCMS: M+H=454.

### Example 32

### Synthesis of (E)-4-hydroxy-3-(3-((1′S,4a′S,8a′S)-5′,5′,8a′-trimethyloctahydro-1′H-spiro[cyclopropane-1,2′-naphthalen]-1′-yl)propylidene)dihydrofuran-2(3H)-one

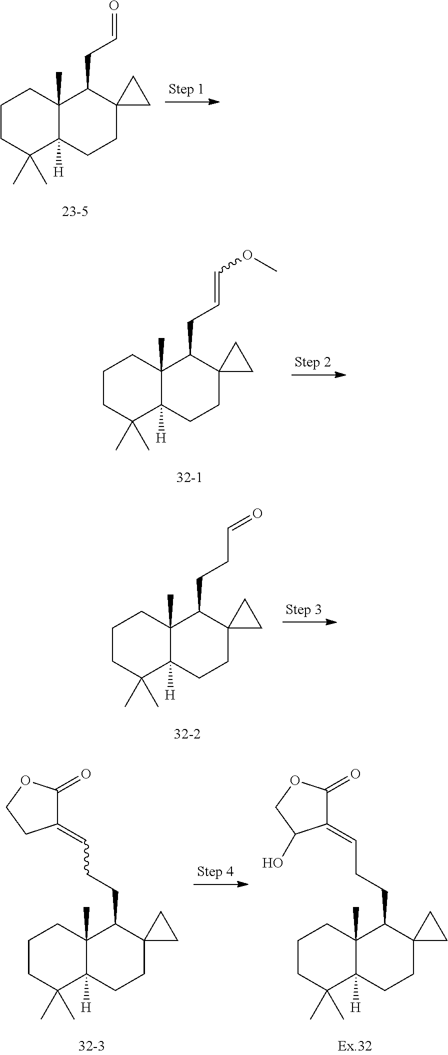 [SMILES: CC1(C)CCC[C@@]2(C)[C@H]1CCC1(CC1)[C@@H]2CC=O ; COC=CC[C@H]1C2(CC[C@H]3C(C)(C)CCC[C@@]31C)CC2 ; CC1(C)CCC[C@@]2(C)[C@H]1CCC1(CC1)[C@@H]2CCC=O ; CC1(C)CCC[C@@]2(C)[C@H]1CCC1(CC1)[C@@H]2CCC=C1CCOC1=O ; CC1(C)CCC[C@@]2(C)[C@H]1CCC1(CC1)[C@@H]2CC/C=C1/C(=O)OCC1O]

Step 1. To a suspension of Ph₃PCH₂OCH₃Cl (67 g, 196 mmol, 3.0 eq.) in dried THF (200 mL) cooled in an ice bath was added t-BuOK (1M in THF, 196 mL, 196 mmol, 3.0 eq.) dropwise. The mixture was stirred at room temperature for 1 h. Aldehyde 23-5 from Example 23 (16.2 g, 65.3 mmol, 1.0 eq.) in anhydrous THF (100 mL) was added to the mixture dropwise at 0° C. The reaction mixture was then stirred at 30° C. for 18 hours. A saturated NH₄Cl aqueous solution was added to the mixture at 0° C., the mixture was extracted with EtOAc (300 mL×3). The combined organic extracts were washed with brine, dried over anhydrous Na₂SO₄ and concentrated to give a crude product, which was purified by column chromatography on silica gel with elution PE: EtOAc=100:1 to give the enol ether 32-1 (8 g, Z/E mixture, 44.3%) as a colorless oil.

¹H NMR (400 MHz, CDCl₃) δ: 6.31 (d, J=12.6 Hz, 0.3H), 5.87 (d, J=6.4 Hz, 0.6H), 4.85-4.71 (m, 0.3H), 4.44-4.31 (m, 0.6H), 3.74-3.57 (m, 3H), 2.06-1.83 (m, 3H), 1.78-1.63 (m, 2H), 1.62-1.44 (m, 4H), 1.41-1.21 (m, 2H), 1.18-1.03 (m, 3H), 1.03-0.89 (m, 9H), 0.73-0.51 (m, 2H), 0.36-0.22 (m, 1H), 0.10-0.06 (m, 1H).

Step 2. To a solution of compound 32-1 (8 g, 29 mmol, 1.0 eq.) in acetone (50 mL) and water (13 mL) was added concentrated HBr (48%, 7 mL) drop-wise while cooling in an ice bath. The reaction mixture was stirred at 30° C. for 18 hours and then poured into water (200 mL). The aqueous mixture was extracted with EtOAc (100 mL×3). The combined organic layers were washed with saturated NaHCO₃, brine, dried with anhydrous Na₂SO₄ and concentrated to give the crude aldehyde 32-2 (6.5 g, 85.5%), which was used for the next step without further purification.

¹H NMR (400 MHz, CDCl₃) δ: 9.76 (s, 1H), 2.56-2.36 (m, 2H), 1.87-1.70 (m, 2H), 1.69-1.56 (m, 3H), 1.52-1.39 (m, 3H), 1.33-1.15 (m, 2H), 1.05-0.93 (m, 3H), 0.93-0.82 (m, 9H), 0.73-0.61 (m, 1H), 0.54 (t, J=6.1 Hz, 1H), 0.30-0.18 (m, 2H), 0.07-0.05 (m, 1H).

Step 3. To a flame dried flask was added LHMDS (1M in THF) (21.3 mL, 21.3 mmol, 1.3 eq), followed by 20 mL of THF. The reaction vessel was cooled to −78° C. and dihydrofuran-2(3H)-one (1.55 g, 18 mmol, 1.1 eq) in THF (10 mL) was added to the above mixture slowly. The reaction mixture was stirred at −78° C. for 1.5 h. Aldehyde 32-2 (4.3 g, 16.4 mmol, 1.0 eq) in THF (10 mL) was added to the solution and the reaction was continued at −78° C. for another 1.5 h. Et₃N (2.48 g, 24.6 mmol, 1.5 eq) and mesyl chloride (2.43 g, 21.3 moml, 1.3 eq) were added successively at 0° C. The reaction mixture was warmed to room temperature and stirred at this temperature for 1.5 h. The mixture was cooled in an ice bath, DBU (3.74 g, 24.6 mmol, 1.5 eq) was added and the reaction was continued for an additional 1 h at 0° C. The reaction mixture was diluted with EtOAc, washed with a saturated aqueous NH₄Cl solution and separated. The aqueous layer was back extracted with EtOAc (100 mL×3) and the combined organic layers were washed with brine, dried over anhydrous Na₂SO₄ and concentrated in vacuum. The residue was purified by silica gel chromatography eluted with PE: EtOAc=50:1 to PE: EtOAc=20:1 to give intermediate 32-3 (3.3 g, Z/E mixture, 60.9%) as colorless oil.

¹HNMR (400 MHz, CDCl₃) δ: 6.81-6.65 (m, 0.76H), 6.31-6.13 (m, 0.17H), 4.49-4.28 (m, 2H), 3.02-2.79 (m, 2H), 2.25-2.05 (m, 2H), 1.88-1.69 (m, 2H), 1.63-1.54 (m, 2H), 1.53-1.38 (m, 3H), 1.37-1.14 (m, 3H), 1.03-0.92 (m, 3H), 0.91-0.78 (m, 9H), 0.57-0.36 (m, 2H), 0.32-0.14 (m, 2H), 0.08-0.08 (m, 1H).

Step 4. To a solution of intermediate 32-3 (2.2 g, 6.66 mmol, 1.0 eq) in dry dioxane (20 mL) was added SeO₂ (0.86 g, 7.66 mmol, 1.15 eq) under N₂ atmosphere at room temperature. Then the reaction mixture was stirred at 110° C. for 18 hours. It was cooled to room temperature and the solvent was concentrated under reduced pressure. The crude product was purified by silica gel chromatography eluted with PE: EtOAc=20:1 to PE: EtOAc=5:1 to give the hydroxylated product with 80% purity. After purification by preparative HPLC (Mobile phase A: water with 0.1% formic acid, Mobile phase B: acetonitrile; Column: Phenomenex Luna 100×21.2 mm×4 um; Detection wavelength: 220 nm), pure compound Ex.32 (200 mg, 4.3% yield) was obtained. Note: this reaction was set in 2 parallel batches and combined during the workup. ¹HNMR (400 MHz, CDCl₃) δ: 7.02-6.81 (m, 1H), 5.14-4.90 (m, 1H), 4.46 (dd, J=6.2, J=10.0 Hz, 1H), 4.36-4.08 (m, 1H), 2.46-2.19 (m, 2H), 2.03-1.66 (m, 3H), 1.59-1.56 (m, 2H), 1.49-1.35 (m, 3H), 1.35-1.23 (m, 2H), 1.23-1.11 (m, 1H), 0.94 (d, J=12.4 Hz, 3H), 0.87 (s, 3H), 0.83 (s, 6H), 0.56-0.38 (m, 2H), 0.32-0.14 (m, 2H), 0.06-−0.08 (m, 1H). The geometry of the double bond was confirmed by noe. HPLC Purity: 100%. LCMS: M+H=347.1.

### Example 33

### Synthesis of N-((S,E)-5-oxo-4-(3-((1′S,4a′S,8a′S)-5′,5′,8a′-trimethyloctahydro-1′H-spiro[cyclopropane-1,2′-naphthalen]-1′-yl)propylidene)tetrahydrofuran-3-yl)formamide

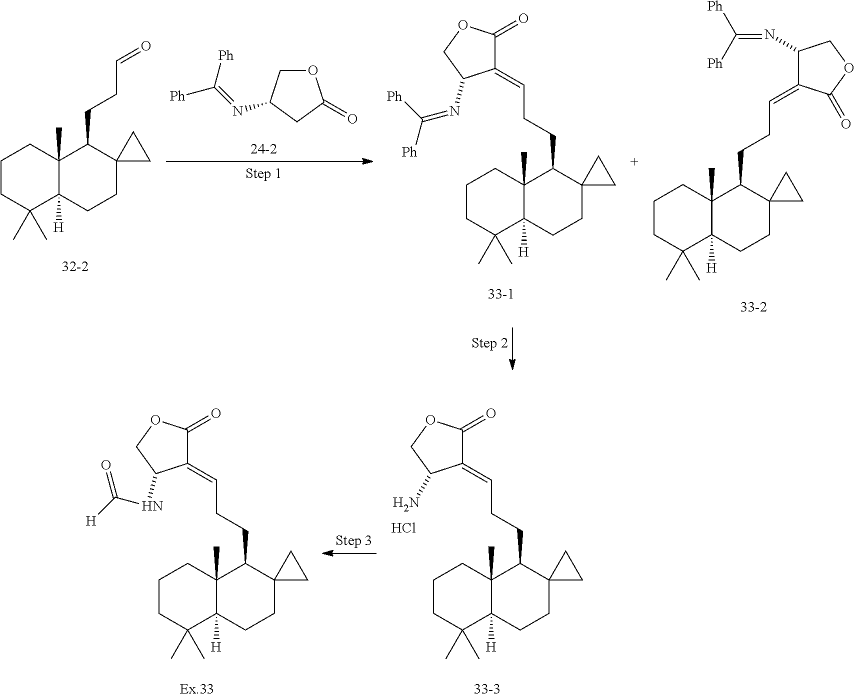

Step 1. To a flame dried flask was added LHMDS (1M in THF) (21.3 mL, 21.3 mmol, 1.3 eq) followed by 20 mL THF. The reaction vessel was cooled to −78° C. and imine 24-2 from Example 24 (4.78 g, 18.03 mmol, 1.1 eq) in THF (10 mL) was added slowly. The reaction mixture was stirred at −78° C. for 1.5 h. Aldehyde 32-2 from Example 32 (4.3 g, 16.4 mmol, 1.0 eq) in THF (10 mL) was added and the resulting mixture was stirred at −78° C. for another 1.5 h. Then Et₃N (2.48 g, 24.6 mmol, 1.5 eq) and mesyl chloride (2.43 g, 21.3 moml, 1.3 eq) were added at 0° C. The reaction mixture was warmed to room temperature and stirred at this temperature for 1.5 h. DBU (3.74 g, 24.6 mmol, 1.5 eq) was added and the reaction was continued for an additional 1 h at 0° C. The mixture was diluted with EtOAc and washed with a saturated aqueous NH₄Cl solution. The aqueous layer was back extracted with EtOAc (100 mL×3) and the combined organic layers were washed with brine, dried over anhydrous Na₂SO₄ and concentrated in vacuum. The residue was purified by silica gel chromatography eluted with PE:EtOAc=50:1 to PE: EtOAc=20:1 to provide the E-isomer 33-1 (3.3 g, 38.3%) and the Z-isomer 33-2 (1.3g, 15.6%) as yellow oils.

33-1: ¹HNMR (400 MHz, CDCl₃) δ: 8.03 (d, J=7.4 Hz, 1H), 7.88-7.78 (m, 2H), 7.77-7.68 (m, 2H), 7.68-7.61 (m, 1H), 7.60-7.51 (m, 2H), 7.43-7.29 (m, 2H), 7.11-6.96 (m, 1H), 4.97 (d, J=2.8 Hz, 1H), 4.63-4.52 (m, 1H), 4.44-4.34 (m, 1H), 2.40-2.25 (m, 1H), 2.19-2.05 (m, 1H), 1.90 (t, J=12.3 Hz, 1H), 1.82-1.69 (m, 3H), 1.66-1.55 (m, 2H), 1.54-1.45 (m, 1H), 1.42-1.27 (m, 3H), 1.09 (m, 3H), 1.07 (s, 3H), 1.02 (s, 3H), 0.98 (s, 3H), 0.68-0.56 (m, 2H), 0.38-0.27 (m, 2H), 0.07-0.02 (m, 1H).

33-2: ¹HNMR (400 MHz, CDCl₃) δ: 7.89 (d, J=7.4 Hz, 2H), 7.77-7.64 (m, 3H), 7.53-7.47 (m, 1H), 7.46-7.39 (m, 2H), 7.29-7.11 (m, 2H), 6.16-6.02 (m, 1H), 4.75 (t, J=6.0 Hz, 1H), 4.42-4.27 (m, 2H), 2.84-2.64 (m, 2H), 1.95-1.26 (m, 7H), 1.25-1.12 (m, 1H), 1.07-0.97 (m, 3H), 0.96-0.91 (m, 6H), 0.88 (s, 6H), 0.56-0.36 (m, 3H), 0.22 (td, J=4.5, 9.0 Hz, 1H), 0.06-0.03 (m, 1H).

Step 2. To a solution of E-isomer 33-1 (3.3 g, 6.48 mmol, 1.0 eq.) in THF (20 mL) cooled in an ice bath was added 1M HCl (20 mL). The mixture was stirred at room temperature for 1.5 h. TLC (PE: EtOAc=3:1) analysis showed there was no starting material remaining in the mixture. Solvent was removed under reduced pressure. The residue was extracted with PE (20 mL×2), and the PE extracts were concentrated to give a mixture of the amine product 33-3 and benzophenone (3 g, crude), which was used directly in the next step.

¹HNMR (400 MHz, DMSO) δ: 8.74 (s, 3H), 7.88-7.71 (m, 6H), 7.70-7.54 (m, 4H), 7.04-6.83 (m, 1H), 4.73 (d, J=5.2 Hz, 1H), 4.58 (dd, J=6.8, J=10.6 Hz, 1H), 4.46 (d, J=10.6 Hz, 1H), 2.38 (q, J=7.4 Hz, 2H), 1.88-1.68 (m, 2H), 1.67-1.54 (m, 2H), 1.44 (d, J=12.0 Hz, 3H), 1.34-1.15 (m, 3H), 1.00 (d, J=10.6 Hz, 3H), 0.93-0.77 (m, 9H), 0.64-0.48 (m, 2H), 0.45-0.34 (m, 1H), 0.32-0.15 (m, 1H), 0.09-0.07 (m, 1H).

Step 3. A mixture of compound 33-3 (1 g, crude, 2.62 mmol, 1.0 eq.) in anhydrous DCM (20 mL) was added Et₃N (1.06 g, 10.48 mmol, 4.0 eq.), HCOOH (0.24 g, 5.25 mmol, 2.0 eq.) and HATU (1.49 g, 3.93 mmol, 1.5 eq.) at 0° C. The reaction mixture was stirred at 28° C. for 18 h. The mixture was poured into water, extracted with DCM (20 mL×3) and the combined organic layers were washed with brine, dried over anhydrous Na₂SO₄ and concentrated in vacuum. The crude product was purified by preparative TLC and recrystallized in PE to give the formamide product Ex.33 (300 mg, 30.6% yield) as a white solid. ¹HNMR (400 MHz, CDCl3) δ: 8.26 (s, 1H), 6.92 (t, J=7.0 Hz, 1H), 6.41 (d, J=7.4 Hz, 1H), 5.35 (t, J=6.8 Hz, 1H), 4.65-4.51 (m, 1H), 4.25 (dd, J=1.6, J=10.2 Hz, 1H), 2.31-2.17 (m, 2H), 1.85-1.74 (m, 1H), 1.73-1.62 (m, 2H), 1.53-1.38 (m, 3H), 1.37-1.25 (m, 2H), 1.20 (dt, J=3.2, J=13.2 Hz, 1H), 1.01-0.92 (m, 3H), 0.91-0.79 (m, 10H), 0.55-0.35 (m, 2H), 0.31-0.13 (m, 2H), 0.02 (td, J=4.8, J=9.2 Hz, 1H). The geometry of the double bond was confirmed by noe. HPLC Purity: 99.1%. LCMS: M+H=374.2.

### Example 34

### Synthesis of N-((S,Z)-5-oxo-4-(3-((l′S,4a′S,8a′S)-5′,5′,8a′-trimethyloctahydro-1′H-spiro[cyclopropane-1,2′-naphthalen]-1′-yl)propylidene)tetrahydrofuran-3-yl)formamide

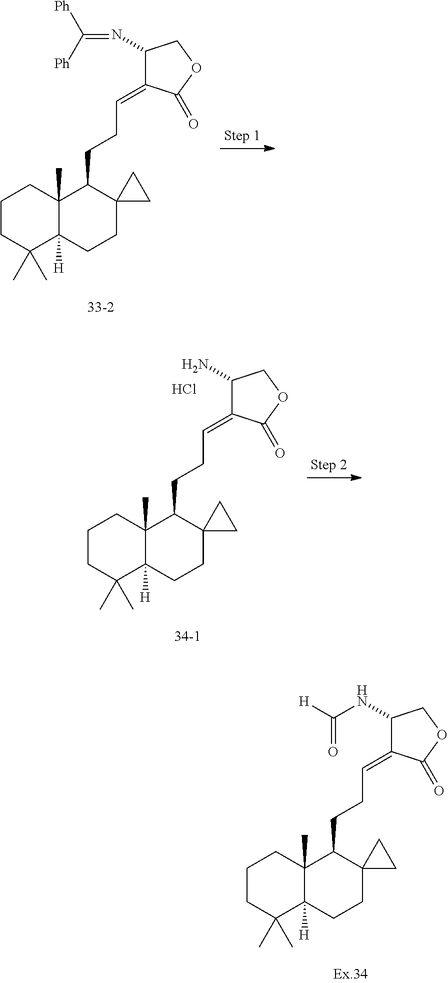 [SMILES: CC1(C)CCC[C@@]2(C)[C@H]1CCC1(CC1)[C@@H]2CC/C=C1\C(=O)OC[C@H]1N=C(c1ccccc1)c1ccccc1 ; CC1(C)CCC[C@@]2(C)[C@H]1CCC1(CC1)[C@@H]2CC/C=C1\C(=O)OC[C@H]1N ; Cl ; CC1(C)CCC[C@@]2(C)[C@H]1CCC1(CC1)[C@@H]2CC/C=C1\C(=O)OC[C@H]1NC=O]

Step 1. To a solution of Z-isomer 33-2 from Example 33 (1.3 g, 2.55 mmol, 1.0 eq.) in THF (10 mL) was added 1M HCl (10 mL) at 0° C. The reaction mixture was stirred at room temperature for 1.5 h. TLC (PE: EtOAc=3:1) showed there was no starting material remaining in the mixture. THF was removed under reduced pressure, and the residue was extracted with EtOAc (30 mL×3). The organic extracts were concentrated to give a mixture of the amine 34-1 and benzophenone (1.3 g, crude), which was used directly in the next step. ¹HNMR (400 MHz, DMSO) δ: 8.76 (s, 3H), 7.93-7.74 (m, 6H), 7.73-7.54 (m, 4H), 7.09-6.82 (m, 1H), 4.73-4.53 (m, 2H), 4.33 (q, J=6.4 Hz, 1H), 2.94-2.73 (m, 1H), 1.91-1.73 (m, 2H), 1.61 (d, J=10.2 Hz, 2H), 1.47 (t, J=13.4 Hz, 3H), 1.38-1.15 (m, 4H), 1.13-0.98 (m, 3H), 0.96-0.79 (m, 10H), 0.59-0.35 (m, 3H), 0.32-0.16 (m, 1H), 0.13-0.08 (m, 1H).

Step 2. A mixture of amine 34-1 (600 mg, crude, 1.57 mmol, 1.0 eq.) in dry DCM (15 mL) was added TEA (480 mg, 4.71 mmol, 3.0 eq.), HCOOH (94 mg, 2.05 mmol, 1.3 eq.) and HATU (900 mg, 2.36 mmol, 1.5 eq.) at 0° C. The reaction mixture was stirred at 28° C. for 18 h. The mixture was poured into water, extracted with DCM (20 mL×3) and the combined organic layers were washed with brine, dried over anhydrous Na₂SO₄ and concentrated in vacuum. The crude product was purified by preparative TLC and recrystallized in PE to give the amide product Ex. 34 (200 mg, 46%) as a white solid. ¹HNMR (400 MHz, CDCl3) δ: 8.30 (s, 1H), 6.57 (t, J=7.2 Hz, 1H), 6.22 (d, J=6.4 Hz, 1H), 5.29 (s, 1H), 4.63 (dd, J=7.4, J=9.8 Hz, 1H), 4.14 (dd, J=3.8, J=9.8 Hz, 1H), 2.93-2.72 (m, 1H), 2.70-2.49 (m, 1H), 1.88-1.70 (m, 3H), 1.67-1.57 (m, 2H), 1.53-1.48 (m, 1H), 1.44 (d, J=6.3 Hz, 1H), 1.37-1.16 (m, 3H), 1.05-0.94 (m, 3H), 0.93-0.83 (m, 9H), 0.52-0.39 (m, 2H), 0.34 (td, J=4.6, J=9.2 Hz, 1H), 0.24 (td, J=4.6, J=9.2 Hz, 1H), 0.02 (td, J=4.8, J=9.2 Hz, 1H). The geometry of the double bond was confirmed by noe. HPLC Purity: 100%. LCMS: M+H=374.1.

### Example 35

### Synthesis of N-((S,E)-5-oxo-4-(3-((1′S,4a′S,8a′S)-5′,5′,8a′-trimethyloctahydro-1′H-spiro[cyclopropane-1,2′-naphthalen]-1′-yl)propylidene)tetrahydrofuran-3-yl)acetamide

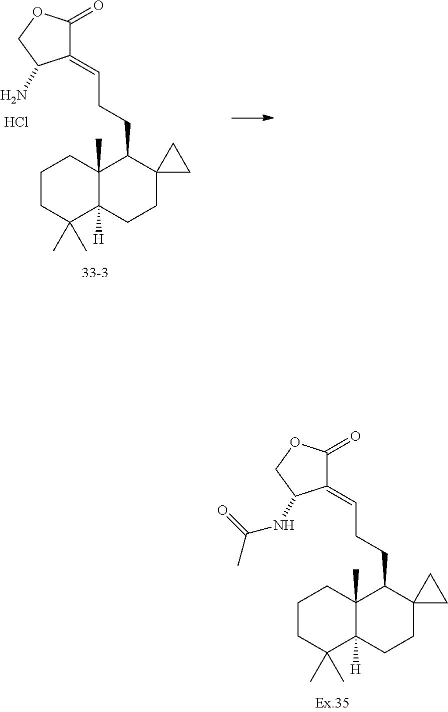 [SMILES: Cl ; CC1(C)CCC[C@@]2(C)[C@H]1CCC1(CC1)[C@@H]2CC/C=C1/C(=O)OC[C@H]1N ; CC(=O)N[C@@H]1COC(=O)/C1=C/CC[C@H]1C2(CC[C@H]3C(C)(C)CCC[C@@]31C)CC2]

To a mixture of amine intermediate 33-3 available from Example 33 (700 mg, crude, 1.84 mmol, 1.0 eq.) in dry DCM (15 mL) was added Et₃N (557 mg, 5.52 mmol, 3.0 eq.) and acetyl chloride (215 mg, 2.75 mmol, 1.5 eq.) at 0° C. The reaction mixture was stirred at room temperature for 1 h. The mixture was poured into saturated NH₄Cl, extracted with DCM (20 mL×3) and the combined organic layers were washed with brine, dried over anhydrous Na₂SO₄ and concentrated in vacuum. The crude product was purified by preparative TLC and recrystallized with PE: EA (5:1) to give the acetamide Ex.35 (265 mg, 37.2%) as a white solid. ¹HNMR (400 MHz, CDCl3) δ: 6.88 (t, J=7.4 Hz, 1H), 6.22 (d, J=6.8 Hz, 1H), 5.21 (t, J=6.4 Hz, 1H), 4.54 (dd, J=6.7, 10.2 Hz, 1H), 4.23 (d, J=10.2 Hz, 1H), 2.31-2.15 (m, 2H), 2.06 (s, 3H), 1.84-1.73 (m, 1H), 1.70 (d, J=12.0 Hz, 1H), 1.64-1.53 (m, 2H), 1.52-1.38 (m, 3H), 1.37-1.25 (m, 2H), 1.19 (dt, J=3.1, 13.1 Hz, 1H), 1.01-0.91 (m, 3H), 0.90-0.80 (m, 9H), 0.52-0.39 (m, 2H), 0.28-0.12 (m, 2H), 0.01 (td, J=4.8, J=9.0 Hz, 1H). HPLC Purity: 99.3%. LCMS: M+H=388.1

### Example 36

### Synthesis of N—((S,Z)-5-oxo-4-(3-((1′S,4a′S,8a′S)-5′,5′,8a′-trimethyloctahydro-1′H-spiro[cyclopropane-1,2′-naphthalen]-1′-yl)propylidene)tetrahydrofuran-3-yl)acetamide

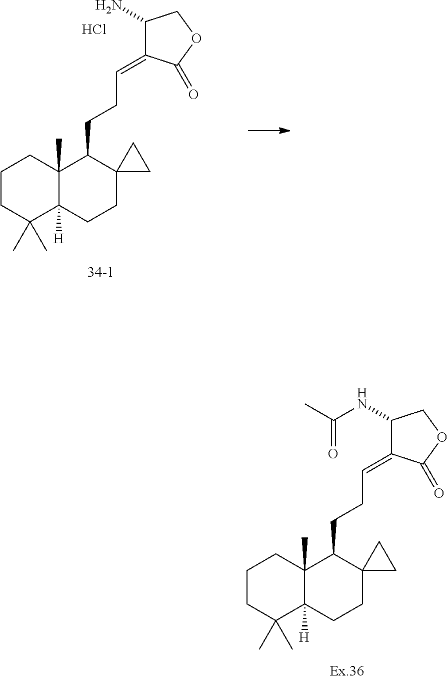 [SMILES: CC1(C)CCC[C@@]2(C)[C@H]1CCC1(CC1)[C@@H]2CC/C=C1\C(=O)OC[C@H]1N ; Cl ; CC(=O)N[C@@H]1COC(=O)/C1=C\CC[C@H]1C2(CC[C@H]3C(C)(C)CCC[C@@]31C)CC2]

To a mixture of amine 34-1 available from Example 34 (300 mg, crude, 0.79 mmol, 1.0 eq.) in dry DCM (10 mL) was added TEA (238 mg, 2.36 mmol, 3.0 eq.) and acetyl chloride (92 mg, 1.19 mmol, 1.5 eq.) at 0° C. The reaction mixture was stirred at room temperature for 1.5 h. The mixture was poured into a saturated NH₄Cl aqueous solution, extracted with DCM (20 mL×3) and the combined organic layers were washed with brine, dried over anhydrous Na₂SO₄ and concentrated in vacuum. The crude product was purified by preparative TLC and then recrystallized (PE: EtOAc=5:1) to give the acetamide product Ex.36 (190 mg, 37.8%) as a white solid. ¹HNMR (400 MHz, CDCl3) δ: 6.53 (t, J=7.4 Hz, 1H), 6.05 (d, J=6.8 Hz, 1H), 5.16 (s, 1H), 4.59 (dd, J=7.4, J=9.8 Hz, 1H), 4.12 (dd, J=3.8, J=10.0 Hz, 1H), 2.88-2.73 (m, 1H), 2.67-2.53 (m, 1H), 2.09 (s, 3H), 1.86-1.68 (m, 2H), 1.66-1.55 (m, 2H), 1.53-1.39 (m, 3H), 1.36-1.26 (m, 2H), 1.26-1.16 (m, 1H), 0.98 (t, J=12.8 Hz, 3H), 0.93-0.83 (m, 9H), 0.52-0.38 (m, 2H), 0.34 (td, J=4.7, 8.9 Hz, 1H), 0.23 (td, J=4.6, J=8.8 Hz, 1H), 0.01 (td, J=4.8, J=9.0 Hz, 1H). HPLC Purity: 99.2%. LCMS: M+H=388.1.

### Example 37

### Synthesis of N—((S,E)-5-oxo-4-(3-((1′S,4a′S,8a′S)-5′,5′,8a′-trimethyloctahydro-1′H-spiro[cyclopropane-1,2′-naphthalen]-1′-yl)propylidene)tetrahydrofuran-3-yl)methanesulfonamide

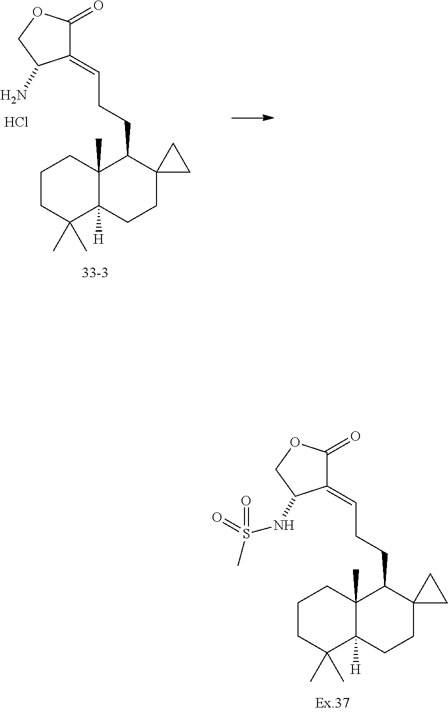 [SMILES: Cl ; CC1(C)CCC[C@@]2(C)[C@H]1CCC1(CC1)[C@@H]2CC/C=C1/C(=O)OC[C@H]1N ; CC1(C)CCC[C@@]2(C)[C@H]1CCC1(CC1)[C@@H]2CC/C=C1/C(=O)OC[C@H]1NS(C)(=O)=O]

To a mixture of amine 33-3 available from Example 33 (700 mg, crude, 1.84 mmol, 1.0 eq.) in dry DCM (15 mL) was added Et₃N (557 mg, 5.52 mmol, 3.0 eq.) and methanesulfonyl chloride (315 mg, 2.75 mmol, 1.5 eq.) at 0° C. The reaction mixture was stirred at room temperature for 1 h. The mixture was poured into a saturated NH₄C1 aq. solution, extracted with DCM (10 mL×3) and the combined organic layers were washed with brine, dried over anhydrous Na₂SO₄ and concentrated in vacuum. The crude product was purified by preparative TLC and recrystallized with PE: EA (5:1) to give the E-sulfonamide Ex.37 (250 mg, 32.1% yield) as a white solid. ¹HNMR (400 MHz, CDCl₃) δ: 6.94 (t, J=7.4 Hz, 1H), 5.03-4.86 (m, 1H), 4.80 (s, 1H), 4.62-4.48 (m, 1H), 4.46-4.31 (m, 1H), 3.06 (s, 3H), 2.42-2.20 (m, 2H), 1.83-1.64 (m, 2H), 1.63-1.51 (m, 2H), 1.49-1.35 (m, 3H), 1.35-1.22 (m, 2H), 1.22-1.08 (m, 1H), 0.93 (d, J=12.6 Hz, 3H), 0.88-0.73 (m, 9H), 0.57-0.39 (m, 2H), 0.29-0.13 (m, 2H), 0.07-0.07 (m, 1H). HPLC Purity: 99.7%. LCMS: M+H=424.1

### Example 38

### Synthesis of N—((S,Z)-5-oxo-4-(3-((1′S,4a′S,8a′S)-5′,5′,8a′-trimethyloctahydro-1′H-spiro[cyclopropane-1,2′-naphthalen]-1′-yl)propylidene)tetrahydrofuran-3-yl)methanesulfonamide

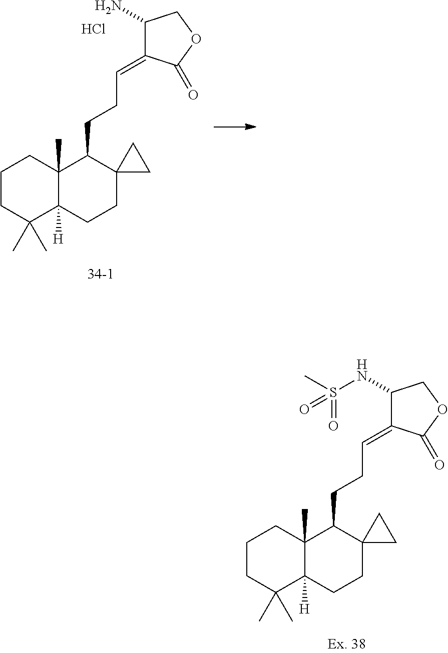 [SMILES: CC1(C)CCC[C@@]2(C)[C@H]1CCC1(CC1)[C@@H]2CC/C=C1\C(=O)OC[C@H]1N ; Cl ; CC1(C)CCC[C@@]2(C)[C@H]1CCC1(CC1)[C@@H]2CC/C=C1\C(=O)OC[C@H]1NS(C)(=O)=O]

To a mixture of amine 34-1 available from Example 24 (200 mg crude, 0.52 mmol, 1.0 eq.) in dry DCM (10 mL) was added Et₃N (159 mg, 1.57 mmol, 3.0 eq.) and methanesulfonyl chloride (89 mg, 0.78 mmol, 1.5 eq.) at ice bath. The reaction mixture was stirred at room temperature for 1.5 h. The mixture was poured into a saturated NH₄Cl aqueous solution, extracted with DCM (20 mL×3) and the combined organic layers were washed with brine, dried over anhydrous Na₂SO₄ and concentrated in vacuum. The crude product was purified by preparative TLC and recrystallized (PE: EtOAc=5:1) to give the Z-sulfonamide product Ex.38 (95 mg, 28.4%) as a white solid. ¹HNMR (400 MHz, CDCl3) δ: 6.61 (t, J=7.6 Hz, 1H), 4.76 (s, 2H), 4.61 (dd, J=7.0, J=9.4 Hz, 1H), 4.27-4.08 (m, 1H), 3.09 (s, 3H), 2.89-2.58 (m, 2H), 1.89-1.70 (m, 2H), 1.60 (d, J=3.6 Hz, 2H), 1.52-1.37 (m, 3H), 1.36-1.26 (m, 2H), 1.26-1.12 (m, 1H), 1.09-0.93 (m, 3H), 0.93-0.79 (m, 9H), 0.53-0.38 (m, 2H), 0.33 (td, J=4.6, J=9.2 Hz, 1H), 0.23 (td, J=4.6, J=9.2 Hz, 1H), 0.09-0.09 (m, 1H). HPLC Purity: 99.7%. LCMS: M+H=424.2.

### Example 39

### Synthesis of N-((E)-4-(2-((4aR,6aS,7R,10aS,10bR)-6a,10b-dimethyl-8-methylenedecahydro-1H-naphtho[2,1-d][1,3]dioxin-7-yl)ethylidene)-5-oxotetrahydrofuran-3-yl)formamide

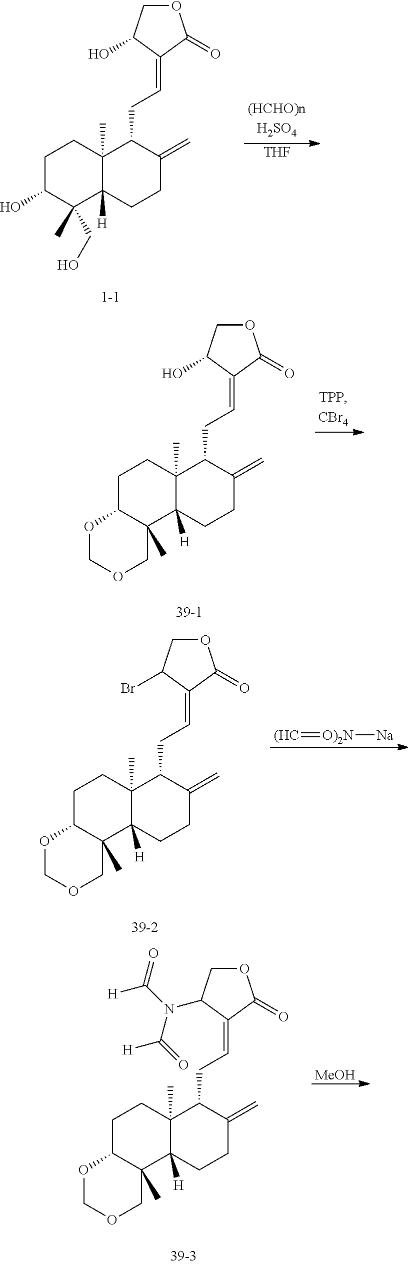

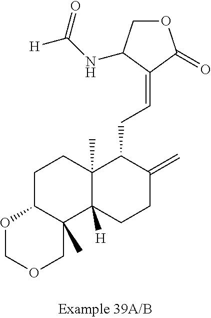 [SMILES: C=C1CC[C@@H]2[C@]3(C)COCO[C@@H]3CC[C@@]2(C)[C@@H]1C/C=C1/C(=O)OCC1NC=O]

Step 1: To a suspension of compound 1 (20 g, 57.1 mmol, 1.0 eq.) in THF (300 mL) was added paraformaldehyde (2.3 g, 76.7 mmol, 1.34 eq), H₂SO₄ (0.8 mL). The mixture was stirred at 70° C. for 1 h. On cooling, the solvent was removed under reduce pressure. The residue was poured into EtOAc (250 mL) with stirring. After filtration, the white solid was dried under reduce pressure to give the target product 39-1 (12 g, 58%) as white solid. HPLC: (Purity: 99.6%) LCMS: (M+H+MeCN: 404.1) ¹HNMR: (400 MHz, CDCl₃) δ: 6.95 (t, J=6.5 Hz, 1H), 5.02 (d, J=5.9 Hz, 1H), 4.97-4.87 (m, 2H), 4.81 (d, J=6.3 Hz, 1H), 4.61 (s, 1H), 4.51-4.41 (m, 1H), 4.30-4.23 (m, 1H), 3.99 (d, J=11.3 Hz, 1H), 3.54-3.39 (m, 2H), 2.65-2.51 (m, 2H), 2.44 (d, J=11.7 Hz, 1H), 2.37-2.23 (m, 1H), 2.08-1.94 (m, 1H), 1.91-1.75 (m, 3H), 1.73-1.62 (m, 1H), 1.41 (s, 3H), 1.31-1.17 (m, 3H), 0.82 (s, 3H).

Step 2: To a solution of 39-1 (6 g, 16.7 mmol, 1.0 eq.) in DCM (80 mL) was added triphenylphosphine (8.5 g, 33.4 mmol, 2.0 eq.), CBr₄ (10.8 g, 33.4 mmol, 2.0 eq.) in one portion at 25° C. The resulting orange solution was stirred at 28° C. for 1 h. After concentration under reduce pressure, the mixture was purified by column chromatography (PE: EA=4:1) to give compound 39-2 (2.4 g, 34%) as white solid. ¹H NMR (400 MHz, CDCl₃) δ: 7.50 (s, 1H), 4.99-4.88 (m, 2H), 4.85 (br. s., 2H), 4.80 (d, J=5.1 Hz, 2H), 4.67 (br. s., 1H), 3.98 (d, J=11.3 Hz, 1H), 3.51 (d, J=9.4 Hz, 1H), 3.42 (d, J=11.3 Hz, 1H), 2.44 (d, J=12.9 Hz, 1H), 2.35-2.20 (m, 1H), 2.18-1.94 (m, 3H), 1.90-1.77 (m, 2H), 1.76-1.61 (m, 2H), 1.46-1.37 (m, 3H), 1.34-1.16 (m, 3H), 0.83-0.74 (m, 3H). LCMS: (M−CHO+H: 390.1)

Step 3: To a solution of compound 39-2 (2.4 g, 6.62 mmol, 1.0 eq) in MeCN (66 mL) was added sodium diformamide (2.5 g, 26.5 mmol, 4.0 eq.). The mixture was stirred at 28° C. for 3 days. After filtration, the filtrate was concentrated. The crude product 39-3 (2.58 g) was obtained as a brown oil and was used for next step directly without purification. Step4: A suspension of compound 39-3 (2.0 g, 4.8 mmol, 1.0 eq) in MeOH (20 mL) was stirred at 28° C. for 1 h. When monitored by TLC (PE:EA=1:1), SM was consumed completely, then the solvent was removed under reduce pressure and the residue was combined with crude product from batch 18940-13-1 (total 3 g, purity 63%), further purified by pre-HPLC (Mobile phase A: water with 0.025% FA, Mobile phase B: acetonitrile; Column: Phenomenex Synergi C18 100×21.2mm×4um; Detection wavelength: 220 nm) to give the target product (0.8 g, 42.7%, purity 94.7%) as white solid. The compound (0.8 g) was further separated by SFC (Mobile phase: Superaritical CO₂/EtOH; Column: Chiralpak AD 250×30 mm×5 um; Detection wavelength: 220 nm) to give 39-B (Rt=8.500 min, 310 mg) as white solid and impure 39 isomer A (0.15 g). The impure 39-A (0.15 g) was further separated by SFC (Mobile phase: Supercritical CO₂/Base-IPA; Column: Chiralpak IC 250×30 mm×5 um; Detection wavelength: 220 nm) to give 39-A (Rt=8.064 min, 81 mg) as a white solid. Data for 39-A: (Purity: 98.7%) LCMS: (M+Na: 412.0) SFC: (ee %: 97.3%) ¹H NMR: (400 MHz, CDCl₃) δ: 8.28 (br. s., 1H), 7.00 (br. s., 1H), 5.89 (br. s., 1H), 5.41 (br. s., 1H), 4.99-4.88 (m, 2H), 4.82 (d, J=5.9 Hz, 1H), 4.57 (t, J=8.2 Hz, 1H), 4.45 (br. s., 1H), 4.22 (d, J=10.2 Hz, 1H), 3.99 (d, J=11.3 Hz, 1H), 3.55-3.40 (m, 2H), 2.58-2.22 (m, 4H), 1.99 (br. s., 1H), 1.82 (d, J=7.4 Hz, 3H), 1.69 (d, J=11.7 Hz, 1H), 1.41 (s, 3H), 1.32-1.14 (m, 3H), 0.80 (s, 3H). Data for 39-B: HPLC: (Purity: 99.8%) LCMS: 18940-26-2C (M+H: 390.0) SFC: (ee %: 96.4%) ¹H NMR: (400 MHz, CDCl₃) δ: 8.6 (br. s., 1H), 6.90 (t, J=6.1 Hz, 1H), 6.14 (br. s., 1H), 5.42 (br. s., 1H), 4.93 (d, J=6.3 Hz, 1H), 4.88 (s, 1H), 4.82 (d, J=6.3 Hz, 1H), 4.64-4.53 (m, 1H), 4.39 (s, 1H), 4.21 (dd, J=1.8, 10.4 Hz, 1H), 3.99 (d, J=11.3 Hz, 1H), 3.56-3.39 (m, 2H), 2.57-2.22 (m, 4H), 2.07-1.93 (m, 1H), 1.91-1.76 (m, 3H), 1.71-1.58 (m, 1H), 1.41 (s, 3H), 1.30-1.12 (m, 3H), 0.81 (s, 3H).

### Example 40

### Synthesis of N-((E)-4-(2-((4aR,6aS,7R,10aS,10bR)-6a,10b-dimethyl-8-methylene-3-oxidodecahydro-1H-naphtho[2,1-d][1,3,2]dioxathiin-7-yl)ethylidene)-5-oxotetrahydrofuran-3-yl)-2-methoxyacetamide

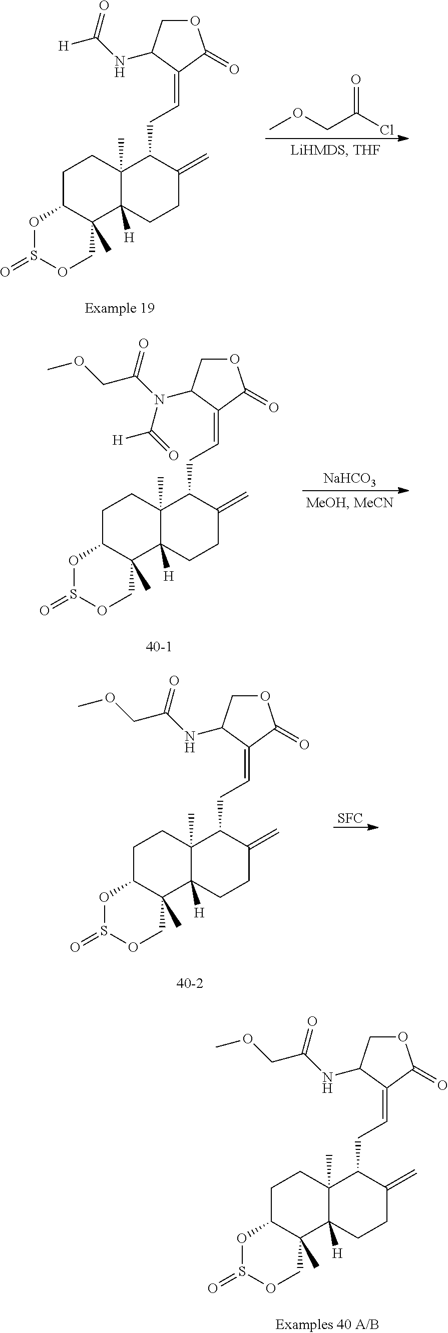

Step 1: To a solution of LiHMDS (2.2 mL, 2.2 mmol, 2.0 eq.) in THF (10 mL) was added a solution of compound Example 19 (450 mg, 1.1 mmol, 1 eq) in THF (5 mL) at −78° C. slowly. The mixture was stirred at −78° C. for 1 hour. Then a solution of 2-methoxyacetyl chloride (237 mg, 2.2 mmol, 2.0 eq.) in THF (5 mL) was added into the mixture dropwise at −78° C. The resulting mixture was stirred at the same temperature for additional 1 h. TLC (PE: EA=1:2) showed the reaction was complete. The reaction mixture was poured into water (200 mL), extracted with EA (20 mL×2). The organic layer was collected, dried over Na₂SO₄, concentrated to give the crude product (0.31 g, 59%) as yellow oil, which was directly used for next step.

Step 2: To a solution of compound 40-1 (310 mg, 0.6 mmol, 1.0 eq.) in MeCN/MeOH (5/5 mL) was added NaHCO₃ (52.5 mg, 0.6 mmol, 1.0 eq.), then the mixture was stirred at 27° C. for 1.5 hours. TLC (PE/EA=1/2) showed the reaction was complete. HCOOH (1 mL) was added to the reaction mixture. And then the solvent was removed in vacuum to give the crude product, which was purified by prep-HPLC (Mobile phase A: 0.225% FA-ACN, Mobile phase B: acetonitrile; Column: Phenomenex Synergi C18 100×21.2 mm×4 um, Detection wavelength: 220 nm) to give compound 40-2 (45 mg, 15% yield) as white solid. ¹HNMR: (400 MHz, CHLOROFORM-d) δ: 6.94-6.87 (m, 1H), 6.80-6.73 (m, 1H), 6.32 (m, 1H), 5.13 (d, J=11.5 Hz, 1H), 4.89 (s, 1H), 4.65-4.50 (m, 1H), 4.45-4.30 (m, 1H), 4.18 (d, J=2.4 Hz, 1H), 3.91 (s, 2H), 3.80-3.75 (m, 1H), 3.42-3.37 (m, 4H), 2.85-2.70 (m, 1H), 2.46-2.25 (m, 3H), 2.20-2.18 (m, 1H), 2.05-1.85 (m, 1H), 1.86-1.75 (m, 3H), 1.42 (s, 3H), 1.29-1.27 (m, 2H), 1.04-0.85 (m, 1H), 0.82 (s, 3H).

Step3: Compound 40-2 (80 mg, 0.16 mmol, 1.0 eq.) was purified by SFC (Mobile phase: Neu-MeOH; Column: AD 250 mm×30 mm, 5 um; Detection wavelength: 220 nm) to give 40-A (Rt=4.114 min, 52 mg, 65%) and 40-B (Rt=4.550 min, 14.2 mg, 17.8%) as white solid.

40-A LCMS: (M+H: 468.0)

¹HNMR: (400 MHz, CHLOROFORM-d) δ: 6.90 (t, J=6.4 Hz, 1H), 6.75 (d, J=8.0 Hz, 1H), 5.38-5.30 (m, 1H), 5.14 (d, J=12.0 Hz, 1H), 4.90 (s, 1H), 4.57-4.46 (m, 1H), 4.45 (s, 1H), 4.20-4.19 (m, 1H), 3.93 (s, 2H), 3.79-3.76 (m, 1H), 3.43-3.39 (m, 4H), 2.85-2.59 (m, 1H), 2.48-2.47 (m, 2H), 2.47-2.25 (m, 1H), 2.20-2.10 (m, 1H), 2.05-1.95 (m, 1H), 1.90-1.80 (m, 2H), 1.79-1.70 (m, 1H), 1.43 (s, 3H), 1.32-1.25 (m, 2H), 1.10-0.95 (m, 1H), 0.83 (s, 3H).

40-B: LCMS: (M+H: 468.0)

¹HNMR: (400 MHz, CHLOROFORM-d) δ: 6.94 (t, J=6.0 Hz, 1H), 6.79 (d, J=8.4 Hz, 1H), 5.33-5.31 (m, 1H), 5.14 (d, J=11.6 Hz, 1H), 4.91 (s, 1H), 4.57-4.54 (m, 1H), 4.52 (s, 1H), 4.20-4.17 (m, 1H), 3.93 (s, 2H), 3.81-3.71 (m, 1H), 3.42-3.38 (m, 4H), 2.75-2.65 (m, 1H), 2.48-2.42 (m, 2H), 2.39-2.25 (m, 1H), 2.20-2.15 (m, 1H), 2.05-1.92 (m, 1H), 1.90-1.70 (m, 3H), 1.43 (s, 3H), 1.40-1.25 (m, 2H), 1.10-0.95 (m, 1H), 0.83 (s, 3H).

### Example 41

### N-((E)-4-(2-((4aR,6aS,7R,10aS,10bR)-6a,10b-dimethyl-8-methylene-3-oxidodecahydro-1H-naphtho[2,1-d][1,3,2]dioxathiin-7-yl)ethylidene)-5-oxotetrahydrofuran-3-yl)-2-hydroxyacetamide

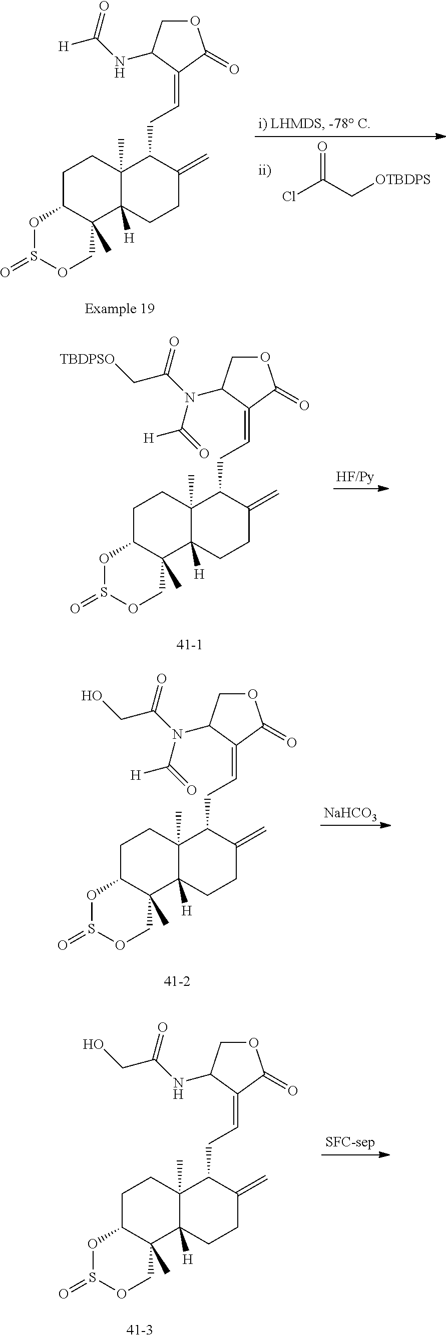

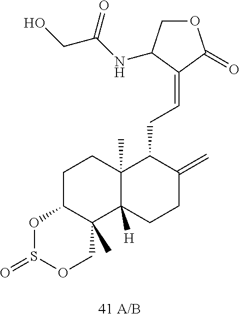

Step 1: To a solution of LiHMDS (1 M in THF, 3.4 mL, 2.2 mmol, 2.0 eq.) was added THF (6 mL) and the mixture was cooled to −78° C. and fulfilled with nitrogen. A suspension of Example 19 (800 mg, 1.89 mmol, 1 eq) in THF (5 mL) was added to the above mixture slowly at −78° C. over 5 min. The mixture was stirred at −78° C. for 1 hour. Next, a suspension of the acid chloride shown above (Li, W. et al., Angew. Chem. Int. Ed, 2009, 48, 8891.) (2.07 mmol, 1.1 eq.) in THF (4 mL) was added into the mixture dropwise at −78° C. The resulting mixture was stirred at the same temperature for additional 1 h. TLC (PE:EA=1:2) showed the new spots, but the starting material was still remained. The reaction mixture was quenched by saturated aqueous NH₄Cl (30 mL), extracted with DCM (30 mL×3). The combined organic layer was dried over Na₂SO₄, filtered and the filtrate was concentrated to give the crude product 41-1 (2.4 g) as brown oil, which was directly used for next step.

Steps 2 and 3: To a solution of compound 41-1 (2.4 g crude, ˜1.89 mmol, 1.0 eq.) in THF (15 mL) was added HF/Py (15 mL) at 0° C. The resulting mixture was stirred at the same temperature for 2 h. Monitored by TLC (PE:EA=1:2). The reaction mixture was poured into water, extracted by EA (30 mL×3). The organic phase was washed by saturated NaHCO₃ (30 mL×4). Then dried over Na₂SO₄, filtered, the filtrate was concentrated and directly used for next step. The residue was dissolved in MeOH (30 mL) and MeCN (5 mL), followed by addition of NaHCO₃ (159 mg, 1.89 mmol, 1 eq). Then the mixture was stirred at 10° C. for 1.5 hours. TLC (EA) showed the reaction was complete. Filtered, the filtrate was concentrated and the residue was purified by prep-HPLC (Mobile phase A: 0.225% FA in water, Mobile phase B: acetonitrile; Column: Phenomenex Synergi C18 100×21.2 mm×4 um, Detection wavelength: 220 nm) to give compound 41-3 (200 mg, isomers mixture, 23.3%) as white solid.

Step 4: Compound 41-3 (200 mg, 0.44 mmol) was purified by SFC (Mobile phase: Neu-MeOH; Column: AS 250 mm×30 mm, 5 um; Detection wavelength: 220 nm) to give 41-A (Rt=5.07 min, 65 mg, 32.5%) and 41-B (Rt=7.1 min, 32 mg, 16%) as white solid.

### Example 41-A

### LCMS: (M+Na: 476)

¹HNMR: (400 MHz, CHLOROFORM-d) δ: 7.02-6.82 (m, 2H), 5.34 (br. s., 1H), 5.14 (d, J=11.5 Hz, 1H), 4.90 (br. s., 1H), 4.64-4.54 (m, 1H), 4.45 (br. s., 1H), 4.28-4.09 (m, 3H), 3.80 (d, J=10.4 Hz, 1H), 3.41 (d, J=11.2 Hz, 1H), 2.79 (q, J=12.8 Hz, 1H), 2.54-2.29 (m, 3H), 2.19 (d, J=10.8 Hz, 1H), 2.01 (d, J=11.9 Hz, 1H), 1.90-1.72 (m, 3H), 1.43 (br. s., 3H), 1.37-1.21 (m, 3H), 1.04 (t, J=12.8 Hz, 1H), 0.83 (br. s., 3H).

### Example 41-B

### (M+Na: 476)

¹HNMR: (400 MHz, CHLOROFORM-d) δ: 6.96 (t, J=6.1 Hz, 1H), 6.86 (d, J=7.9 Hz, 1H), 5.34 (br. s., 1H), 5.14 (d, J=11.5 Hz, 1H), 4.92 (s, 1H), 4.56 (dd, J=7.1, 10.1 Hz, 1H), 4.48 (s, 1H), 4.25-4.16 (m, 3H), 3.80 (dd, J=4.1, 12.7 Hz, 1H), 3.40 (d, J=11.2 Hz, 1H), 2.88-2.74 (m, 1H), 2.47 (d, J=14.3 Hz, 2H), 2.40-2.19 (m, 3H), 2.07-1.95 (m, 1H), 1.88-1.77 (m, 3H), 1.43 (s, 3H), 1.36-1.23 (m, 3H), 1.06 (t, J=13.6 Hz, 1H), 0.83 (s, 3H).

### Example 42

### Synthesis of (S,E)-4-hydroxy-3-(2-((1R,4aR,5R,6S,8aR)-6-hydroxy-5,8a-dimethyl-2-methylene-6-(trifluoromethyl)decahydronaphthalen-1-yl)ethylidene)dihydrofuran-2(3H)one and (S,E)-4-hydroxy-3-(2-((1R,4aR,5R,6R,8aR)-6-hydroxy-5,8a-dimethyl-2-methylene-6-(trifluoromethyl)decahydronaphthalen-1-yl)ethylidene)dihydrofuran-2(3H)-one.

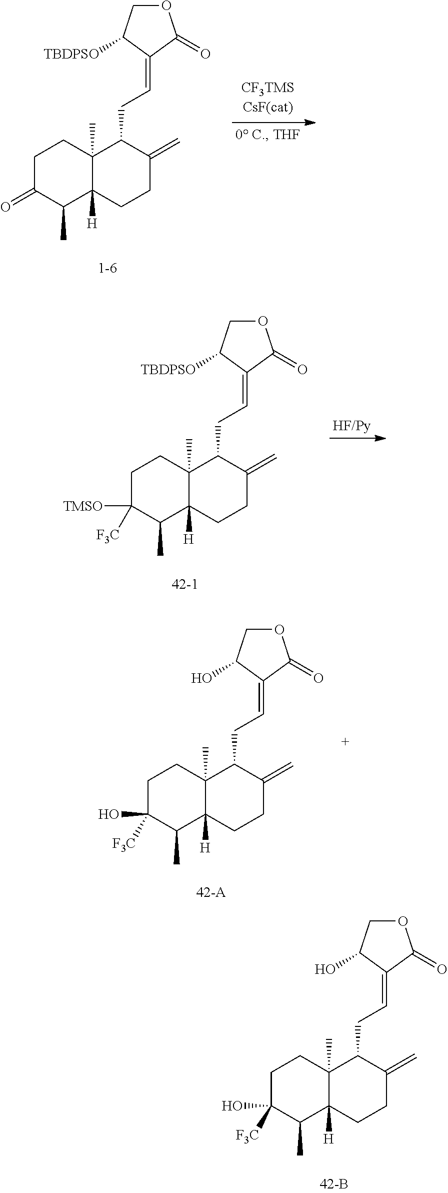

Step 1: To a solution of compound 1-6 (5 g, 9.0 mmol, 1.0 eq.) in THF (80 mL) was added TMSCF₃ (3.8 g, 27.0 mmol, 3.0 eq.) and CsF (0.14 g, 0.9 mmol, 0.1 eq.) at −78° C. under N₂. The reaction mixture was stirred at −78° C. for 3 h. TLC (PE/EA=3:1) showed the starting material was remained and a new spot was detected. Saturated NH₄Cl (10 mL) was added to quench the reaction. The two phases were separated, and the aqueous phase was extracted with EtOAc (20 mL×3). The combined organic phase was dried over Na₂SO₄, filtered, concentrated to give a crude product, which was purified by column chromatography (PE/EA=10:1) to give compound 42-1 (4.8 g, 76.4% yield) as white solid.

¹HNMR: (400 MHz, CDCl₃) δ: 7.71-7.69 (m, 4H), 7.49-7.40 (m, 6H), 6.92-6.88 (m, 1H), 5.05-4.99 (m, 1H), 4.91 (s, 1H), 4.53 (s, 1H), 4.20-4.12 (m, 1H), 4.02-3.99 (m, 1H), 2.40-2.22 (m, 3H), 2.02-1.85 (m, 1H), 1.75-1.56 (m, 5H), 1.19-0.98 (m, 16H), 0.50 (s, 3H), 0.19 (s, 9H), 0.14 (s, 3H).

Step 2: To a solution of compound 42-1(4.8 g, 6.9 mmol, 1.0 eq.) in THF (70 mL) was added HF/Py (70 mL) at 0° C. The reaction mixture was stirred at 25° C. for 3 h. TLC (PE/EA=8:1) showed the starting material was consumed. Water (50 mL) was added, followed by EtOAc (50 mL). The two phases were separated, and the aqueous phase was extracted with EtOAc (30 mL×2). The combined organic phase was dried over Na₂SO₄, filtered, concentrated to give a crude product, which was purified by column chromatography on silica gel (PE: EA=5:1 to 1:1) to give a crude compound, then re-purified by prep. HPLC (Mobile phase A: water with 0.225% Formic acid, Mobile phase B: acetonitrile; Column: Phenomenex Synergi C18 100×21.2 mm×4 um wavelength: 220 nm) to give 42-A (200 mg, 7.5%) and 42-B (450 mg, 16.9%) as white solid.

Spectra of 42-A: ¹HNMR: (400 MHz, CDCl₃) δ: 7.01 (t, J=6.4Hz, 1H), 5.05 (br s, 1H), 4.95 (s, 1H), 4.63 (s, 1H), 4.49-4.45 (m, 1H), 4.27 (dd, J=1.6, 11.8 Hz, 1H), 2.63-2.61 (m, 2H), 2.43-2.41 (m, 1H), 2.01-1.70 (m, 7H), 1.69-1.52 (m, 3H), 1.13-0.99 (m, 1H), 0.98 (d, J=6.4 Hz, 3H), 0.73 (s, 3H). LCMS: (M+H: 389.1).

Spectra of 42-B ¹HNMR: (400 MHz, CDCl₃) δ: 7.01 (t, J=6.4Hz, 1H), 5.04 (br s, 1H), 4.96 (s, 1H), 4.63 (s, 1H), 4.50-4.46 (m, 1H), 4.27 (dd, J=1.6, 11.8 Hz, 1H), 2.63-2.58 (m, 2H), 2.43-2.40 (m, 1H), 2.20-2.17 (m, 2H), 1.98-1.79 (m, 3H), 1.79-1.46 (m, 6H), 1.15-1.04 (m, 1H), 1.03 (d, J=6.4 Hz, 3H), 0.73 (s, 3H). LCMS: (M+H: 389.1).

### Example 43

### Synthesis of N-((E)-4-(2-((1R,4aR,5R,6R,8aR)-6-hydroxy-5,8a-dimethyl-2-methylene-6-(trifluoromethyl)decahydronaphthalen-1-yl)ethylidene)-5-oxotetrahydrofuran-3-yl)formamide

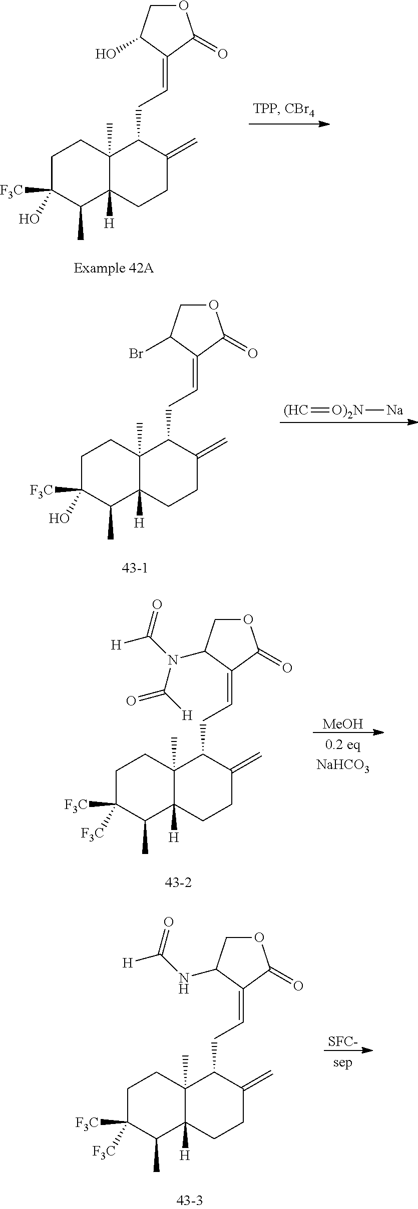

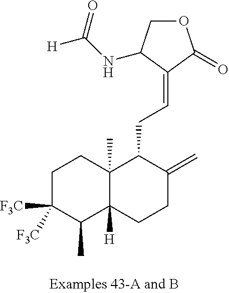

Step 1: To a solution of compound 42-B (1.5 g, 3.86 mmol, 1.0 eq.) in DCM (150 mL) was added TPP (2.53 g, 9.65 mmol, 2.5 eq.) and CBr₄ (3.2 g, 9.65 mmol, 2.5 eq.). The resulting suspension was stirred at 25° C. for 1.5 h. TLC (PE: EA=1:1) showed new spot and a little starting material remained. Saturated NaHCO₃ (30 mL) was added, and the two phases were separated. The aqueous phase was extracted with DCM (30 mL×2). The combined organic phase was dried over Na₂SO₄, filtered, concentrated to give a crude product, which was purified by pep.TLC (PE: EA=10:1 to 2:1) to give compound 43-1 (1.3 g, 74.7%) as colorless oil.

Step 2: A suspension of compound 43-1 (1.3 g, 2.88 mmol, 1.0 eq.) in MeCN (100 mL) was added diformylimide sodium salt (1.09 g, 11.5 mmol, 4.0 eq.). The mixture was stirred at 25° C. for 48 h. TLC (PE: EA=2:1) showed the starting material remained and a new spot detected. The solvent was concentrated. EtOAc (50 mL) and H₂O (20 mL) were added. The two phases were separated, and the aqueous phase was extracted with EtOAc (20 mL×2). The combined organic phase was dried over Na₂SO₄, filtered, concentrated to give a crude product 43-2, which was used for the next step without any further purification.

Step3: A suspension of compound 43-2 (1.1 g, 2.48 mmol, 1.0 eq.) in MeOH (100 mL) was added NaHCO₃ (43 mg, 0.5 mmol, 0.2 eq.) the mixture was stirred at 25° C. for 1 h. TLC (PE: EA=1:2) showed compound 2 was consumed. HCOOH (0.2 mL) was added to neutralize the reaction. The solvent was concentrated to give a crude product, which was purified by column chromatography (PE/EA=3:1 to 1:2), then by prep. HPLC (Mobile phase A: water with 0.225% FA, Mobile phase B: acetonitrile; Column: Phenomenex Synergi C18 100×21.2 mm×4 um; Detection wavelength: 220 nm) to give a crude product (100 mg, with two isomers) as a white solid. Further purification by SFC (Mobile phase: 35% MeOH, 80 ML/MIN, Column: AD (250 mm×30 mm, 10 um); Detection wavelength: 220 nm) to give 43A (114.6 mg, 11.1%) as white solid and 43B (100.1 mg, 9.7%) as white solid.

Spectra of 43A: LCMS: (M+H: 416.1)

¹H NMR: (400 MHz, CDCl₃) δ: 8.25 (s, 1H), 6.93 (t, J=6.0 Hz, 1H), 6.19 (d, J=7.2 Hz, 1H), 5.42 (br s, 1H), 4.92 (s., 1H), 4.64-4.56 (m, 1H), 4.42 (s, 1H), 4.21 (d, J=10.4 Hz, 1H), 2.49-2.30 (m, 4H), 2.20-2.16 (m, 1H), 2.02-1.96 (m, 2H), 1.80-1.75 (m, 4H), 1.49-1.43 (m, 1H), 1.05-1.03 (m, 4H), 0.77 (s, 3H).

Spectra of 43B : LCMS: (M+H: 416.1)

¹H NMR: (400 MHz, CDCl₃) δ: 8.26 (s, 1H), 6.99 (t, J=6.0 Hz, 1H), 6.16 (d, J=7.2 Hz, 1H), 5.41 (br s, 1H), 4.94 (s., 1H), 4.60-4.56 (m, 1H), 4.45 (s, 1H), 4.22 (d, J=10.4 Hz, 1H), 2.51-2.28 (m, 4H), 2.22-2.18 (m, 1H), 1.95-1.80 (m, 1H), 1.80-1.75 (m, 4H), 1.45-1.41 (m, 1H), 1.09-1.03 (m, 4H), 0.76 (s, 3H).

### Example 44

Synthesis of N-((E)-4-(2-((1R,4aR,5R,6S,8aR)-6-hydroxy-5,8a-dimethyl-2-methylene-6-(trifluoromethyl)decahydronaphthalen-1-yl)ethylidene)-5-oxotetrahydrofuran-3-yl)formamide. Examples 44A and B were made starting from Example 42A in a similar fashion to the method described above for the synthsis Example 43A/B.

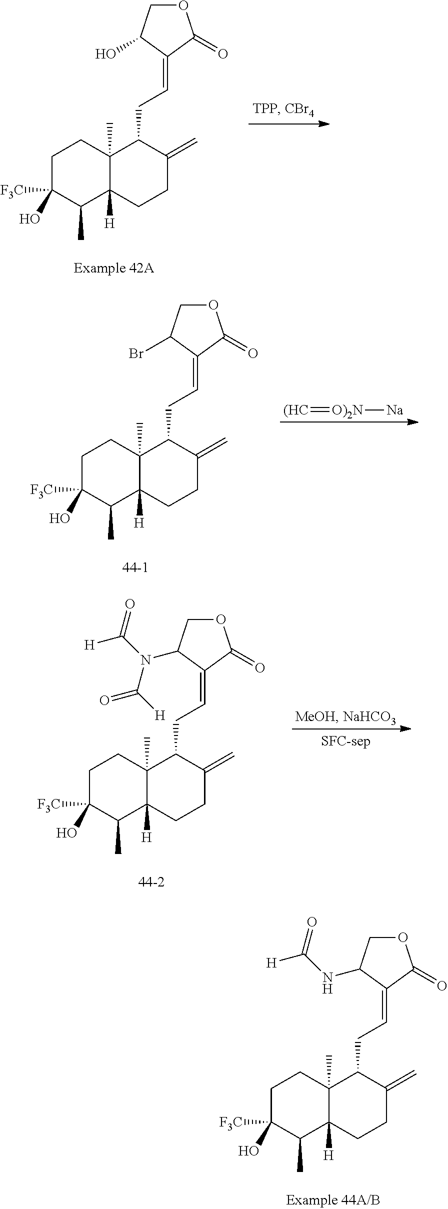

Final purification by SFC (Mobile phase: 35% i-PrOH, 50 mL/min, Column: AS (250 mm×30 mm, 5 um); Detection wavelength: 220 nm) to give 44A (20.1 mg, 40.2% separated yield) as white solid and 44B (10.8 mg, 21.6% separated yield) as white solid.

Spectra of 44A: LCMS: (M+H: 416.1)¹H NMR: 18948-82-1A (400 MHz, CDCl₃) δ: 8.19 (s, 1H), 6.87 (t, J=6.0 Hz, 1H), 5.92 (br s, 1H), 5.35 (m, 1H), 4.85 (s., 1H), 4.53-4.49 (m, 1H), 4.35 (s, 1H), 4.16-4.13 (m, 1H), 2.49-2.33 (m, 3H), 1.93-1.78 (m, 3H), 1.75-1.68 (m, 4H), 1.56-1.42 (m, 2H), 1.05-1.03 (m, 1H), 0.90 (d, J=6.8 Hz, 3H), 0.63 (s, 3H).

Spectra for 44B: LCMS: (M+H: 416.1) ¹H NMR: 18948-82-2b (400 MHz, CDCl₃) δ: 8.26 (s, 1H), 7.01 (t, J=6.0 Hz, 1H), 6.10 (d, J=7.6 Hz, 1H), 5.41 (br s, 1H), 4.93 (s., 1H), 4.59-4.54 (m, 1H), 4.47 (s, 1H), 4.23-4.20 (m, 1H), 2.53-2.40 (m, 3H), 2.01-1.78 (m, 4H), 1.77-1.52 (m, 5H), 1.11-0.99 (m, 1H), 0.98 (d, J=6.4 Hz, 1H), 0.69 (s, 3H).

### Example 45

### Synthesis of N-((E)-4-(2-((4aR,6aS,7R,10aS,10bR)-6a,10b-dimethyl-8-methylenedecahydro-1H-naphtho[2,1-d][1,3]dioxin-7-yl)ethylidene)-5-oxotetrahydrofuran-3-yl)acetamide

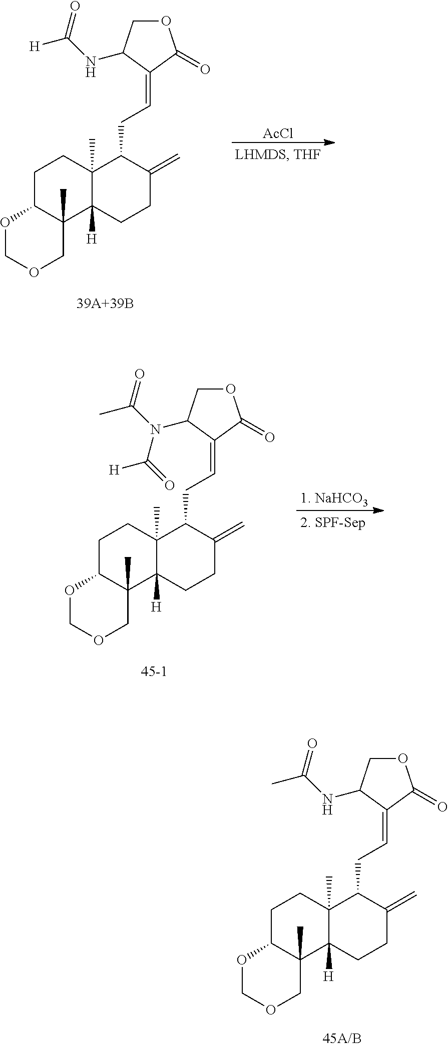

Step 1: To a solution of LiHMDS (1M in THF, 8.1 mL, 8.1 mmol, 1.5 eq) was added a mixture of Example 39-A and 39-B (2.1 g, 5.4 mmol, 1 eq) in THF (50 mL) drop wise at −78° C. under N₂ atmosphere. The resulting mixture was stirred at −78° C. for 1 h. Acetyl chloride (550 mg, 7.0 mmol, 1.3 eq) was added via syringe. The reaction mixture was stirred for additional 1 h. TLC (PE/EA=1:3) indicated the starting material still remained. Quenched by saturated NH₄Cl (30 mL). The solvent (THF) was removed under reduce pressure and the water layer was extracted with EtOAc (20 mL×3). The combined organic layer was washed with brine (30 mL×3), dried over Na₂SO₄, filtered and concentrated. The residue was purified by column chromatography (PE: EA=1:1) to give compound 45-1 (1.5 g, 64%) as white solid.

¹HNMR: (400 MHz, CDCl₃) δ: 9.15 (s, 1H), 6.83-6.65 (m, 1H), 6.05-6.01 (m, 1H), 4.97-4.89 (m, 1H), 4.86 (s, 1H), 4.81 (d, J=6.2 Hz, 1H), 4.62-4.53 (m, 1H), 4.37 (s, 1H), 4.18-4.13 (m, 1H), 3.97 (d, J=11.2 Hz, 1H), 3.53-3.37 (m, 2H), 2.50-2.46 (m, 3H), 2.41 (d, J=12.8 Hz, 1H), 2.34-2.20 (m, 2H), 2.17 (t, J=7.1 Hz, 1H), 2.01-1.90 (m, 1H), 1.84-1.74 (m, 3H), 1.71-1.58 (m, 2H), 1.40 (s, 3H), 1.20 (br. s., 2H), 0.78-0.72 (m, 3H).

Step 2 : To a solution of compound 45-1 (1.5 g, 3.48 mmol, 1 eq) in MeOH (30 mL) was added NaHCO₃ (58 mg, 0.70 mmol, 0.2 eq). The mixture was stirred at 20° C. for 40 min. TLC (PE/EA=1:2) indicated the completion. The reaction mixture was neutralized with FA to pH=5-6 and the solvent (MeOH) was removed under reduced pressure. The residue was purified by column (PE: EA=1:1) to give the desired product (0.8 g, 57.1%) as white solid. The compound (800 mg) was further separated by SFC (Mobile phase: Supercritical CO₂/MeOH; Column: Chiralpak AD 250×30 mm×5 um; Detection wavelength: 220 nm) to give diastereoisomer 45-A ((Rt=2.975 min, 344 mg) as white solid, diastereoisomer 45-B (200 mg, purity 95.6%) as white solid. Further purification by prep-HPLC (Mobile phase A: water with 0.225% FA, Mobile phase B: acetonitrile; Column: Phenomenex Synergi C18 100×21.2 mm×4 um; Detection wavelength: 220 nm) gave 45-B (Rt=3.506 min 115.2 mg) as white solid.

Data for 45-A LCMS: (M+H: 404.1)

¹HNMR: (400 MHz, CDCl₃) δ: 6.88 (t, J=6.3 Hz, 1H), 6.04 (br. s., 1H), 5.32 (t, J=6.7 Hz, 1H), 4.93 (d, J=6.4 Hz, 1H), 4.88 (s, 1H), 4.82 (d, J=6.4 Hz, 1H), 4.54 (dd, J=6.8, 10.1 Hz, 1H), 4.41 (s, 1H), 4.19 (dd, J=1.8, 10.1 Hz, 1H), 3.99 (d, J=11.2 Hz, 1H), 3.54-3.40 (m, 2H), 2.58-2.22 (m, 4H), 2.04 (s, 3H), 2.02-1.92 (m, 1H), 1.89-1.77 (m, 3H), 1.72-1.63 (m, 1H), 1.41 (s, 3H), 1.30-1.10 (m, 3H), 0.80 (s, 3H).

Data for 45-B: LCMS: (M+H: 404.1)

¹HNMR: (400 MHz, CDCl₃) δ: 6.90 (t, J=5.8 Hz, 1H), 6.39 (d, J=7.7 Hz, 1H), 5.31 (br. s., 1H), 4.93 (d, J=6.2 Hz, 1H), 4.88 (br. s., 1H), 4.81 (d, J=6.2 Hz, 1H), 4.52 (dd, J=7.2, 10.0 Hz, 1H), 4.46 (s., 1H), 4.19 (d, J=10.1 Hz, 1H), 3.98 (d, J=11.2 Hz, 1H), 3.53-3.39 (m, 2H), 2.54-2.39 (m, 2H), 2.37-2.21 (m, 2H), 2.04 (s, 3H), 2.02-1.93 (m, 1H), 1.89-1.77 (m, 3H), 1.68-1.63 (m, 1H), 1.40 (s, 3H), 1.30-1.14 (m, 3H), 0.79 (s, 3H).

### Example 46

### Synthesis of N-((E)-4-(2-((4aR,6aS,7R,10aS,10bR)-6a,10b-dimethyl-8-methylenedecahydro-1H-naphtho[2,1-d][1,3]dioxin-7-yl)ethylidene)-5-oxotetrahydrofuran-3-yl)-2-methoxyacetamide

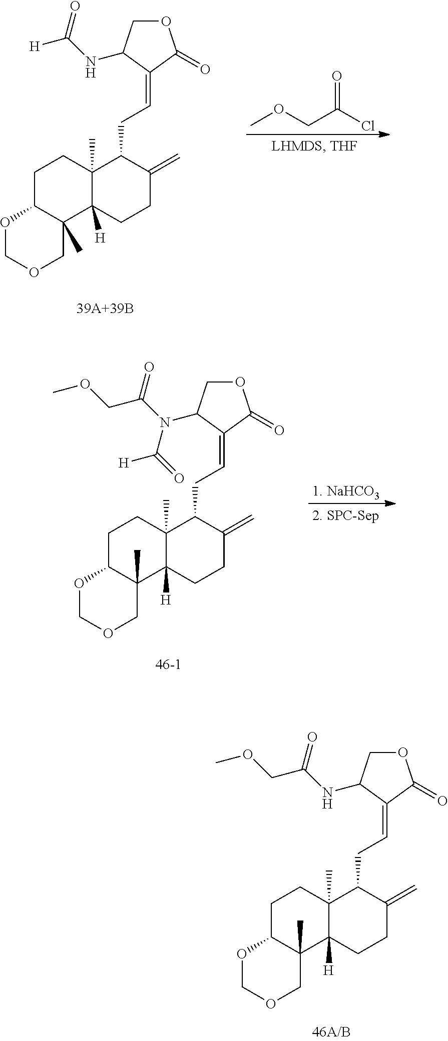

Step 1: To a solution of LiHMDS (7.7 mL, 1.0 M in THF, 2.0 eq) in anhydrous THF (20 mL) was added compounds 39A+39B (1.5 g, 3.85 mmol, 1.0 eq) in anhydrous THF (5 mL) drop-wise under N₂ atmosphere. The resulting yellow solution was stirred at −78° C. After 1 hour, 2-methoxyacetyl chloride (835.90 mg, 7.7 mmol, 2.0 eq) in THF (2 mL) was added. The reaction mixture was stirred at −78° C. for another 1 hour. TLC (PE:EA=1:5) showed the starting material was consumed completely. The reaction mixture was quenched with sat. NH₄Cl (20 mL), the organic layer was separated and the aqueous phase was extracted with EtOAc (40 mL) twice. The combined organic layers were washed with brine (50 mL), dried over Na₂SO₄, filtered, concentrated to give compound 46-1 (1.6 g, crude) as white solid. LCMS: (M+Na: 484.1)

Step 2: To a suspension of compound 46-1 (1.8 g, 3.9 mmol, 1.0 eq) in MeOH (80 mL) was added NaHCO₃ (65.52 mg, 0.78 mmol, 0.2 eq). The reaction mixture was stirred at 15° C. for 2 hours. TLC (PE:EA=1:5) showed the starting material was consumed completely. 40 mg of HCOOH was added. The resulting mixture was concentrated to dryness to give the crude product. The residue was purified by prep. HPLC (column: Agela ASB 150×25 mm×5 um, gradient: 34-64% B (A=water/0.225% FA, B=acetonitrile), flow rate: 25 mL/min) to give the desired product, then lyophilized to obtain 550 mg of a mixture of Examples 46A and 46B as white solid. Compounds 46A and 46B (550 mg, 1.27 mmol, 1.0 eq) were separated by SFC (column: AD (250 mm×30 mm, 5 um), condition: Neu-EtOH, flow rate: 65 mL/min) to give the compound 46A (Rt=2.715 min, 310 mg, 99.4% purity) and 46B (Rt=3.871 min, 120 mg, 93.82% purity) both as white solid. 120 mg of 46B was purified by prep. HPLC (column: Agela ASB 150×25 mm×5 um, gradient: 31-61% B (A=water/0.225% FA, B=acetonitrile), flow rate: 25 mL/min) to give the desired product, then lyophilized to obtain 102 mg of 46B as white solid.

Spectra for 46A:LCMS: (M+H: 434.0) ¹HNMR: (400 MHz, CDCl₃) δ: 6.95 (t, J=6.4 Hz, 1H), 6.78 (d, J=7.6 Hz, 1H), 5.34 (t, J=6.8 Hz, 1H), 4.92 (d, J=6.4 Hz, 1H), 4.88 (s, 1H), 4.81 (d, J=6.4 Hz, 1H), 4.56 (dd, J=10.0, 7.0 Hz, 1H), 4.44 (s, 1H), 4.18 (dd, J=10.2, 2.2 Hz, 1H), 3.97 (d, J=11.2 Hz, 1H), 3.93 (s, 2H), 3.50-3.44 (m, 1H), 3.43 (s, 3H), 3.41 (br. s., 1H), 2.49-2.38 (m, 2H), 2.35-2.32 (m, 1H), 2.30-2.22 (m, 1H), 2.01-1.92 (m, 1H) 1.85-1.74 (m, 3H), 1.66-1.60 (m, 1H), 1.40 (s, 3H), 1.25-1.19 (m, 2H), 1.18-1.10 (m, 1H), 0.78 (s, 3H).

Spectra for 46B LCMS: (M+H: 434.1) ¹HNMR: (400 MHz, CDCl₃) δ: 6.97 (t, J=6.2 Hz, 1H), 6.83 (d, J=7.6 Hz, 1H), 5.36 (t, J=6.8 Hz, 1H), 4.93 (d, J=6.4 Hz, 1H), 4.88 (s, 1H), 4.82 (d, J=6.4 Hz, 1H), 4.56 (dd, J=10.0, 7.2 Hz, 1H), 4.44 (s, 1H), 4.20 (dd, J=10.4, 2.0 Hz, 1H), 3.98 (d, J=11.2 Hz, 1H), 3.94 (s, 2H), 3.49-3.42 (m, 2H), 3.43 (s, 3H), 2.52-2.41 (m, 2H), 2.38-2.31 (m, 1H), 2.31-2.22 (m, 1H), 1.99 (br. s., 1H), 1.84 (d, J=12.4 Hz, 3H), 1.73-1.65 (m, 1H), 1.41 (s, 3H), 1.26-1.16 (m, 3H), 0.78 (s, 3H).

### Examples 47 and 48

### Synthesis of (S,E)-4-hydroxy-3-(2-((1R,4aR,5R,6S,8aR)-6-hydroxy-2,5,8a-trimethyl-6-(trifluoromethyl)-1,4,4a,5,6,7,8,8a-octahydronaphthalen-1-yl)ethylidene)dihydrofuran-2(3H)-one and (S,E)-4-hydroxy-3-(2-((1R,4aR,5R,6R,8aR)-6-hydroxy-2,5,8a-trimethyl-6-(trifluoromethyl)-1,4,4a,5,6,7,8,8a-octahydronaphthalen-1-yl)ethylidene)dihydrofuran-2(3H)-one

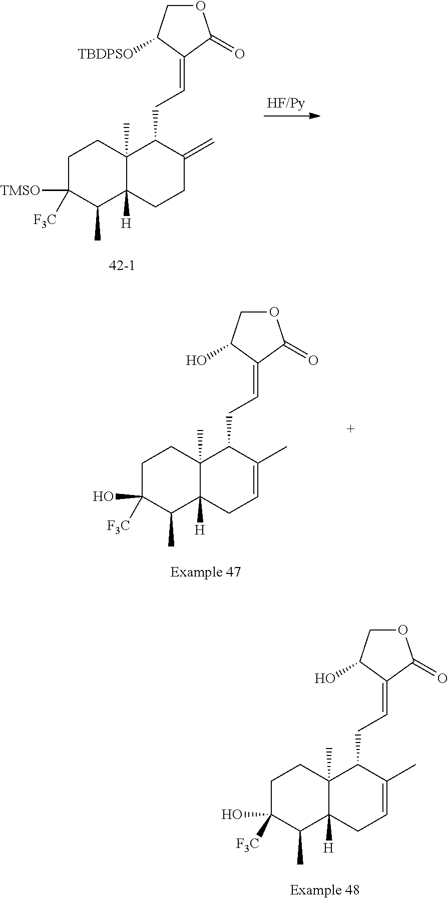

Step 1: To a solution of compound 42-1 (8 g, 11.4 mmol, 1.0 eq.) in THF (80 mL) was added HF/Py (80 mL) at 0° C. The reaction mixture was stirred at 25° C. for 3 h. TLC (PE/EA=8:1) showed the starting material was consumed. EtOAc (100 mL) was added, followed by H₂O (100 mL). The two phases were separated, and the aqueous phase was extracted with EtOAc (30 mL×2). The combined organic phase was concentrated. Then the two phases were separated again. The aqueous phase was extracted with EtOAc (30 mL×2). The combined organic phase was dried over Na₂SO₄, filtered, concentrated to give a crude product, which was purified by column chromatography on silica gel (PE: EA=8:1 to 1:1) to give a crude compound, then purified by prep.HPLC (Mobile phase A: water with 0.225% Formic acid, Mobile phase B: acetonitrile; Column: Phenomenex Synergi C18 100×21.2 mm×4 um wavelength: 220 nm) to give Example 47 (121.3 mg, 2.7%) and Example 48 (200 mg, 4.5%) as white solid. 200 mg of Example 48 was further purified by SFC (Mobile phase: 20% EtOH, 50 ML/MIN, Column: OJ (250 mm×30 mm, 5 um); Detection wavelength: 220 nm) to give Example 48 (125.1 mg, 62.6% separated yield) as white solid.

Spectra of 47 :¹HNMR: (400 MHz, CDCl₃) δ: 7.14 (t, J=6.4 Hz, 1H), 5.50 (br s, 1H), 5.08 (m, 1H), 4.49 (dd, J=6.0, 10.4 Hz, 1H), 4.27 (dd, J=1.6, 10.4 Hz, 1H), 2.68-2.60 (m, 2H), 2.25-2.02 (m, 3H), 1.82-1.71 (m, 4H), 1.69 (s, 3H), 1.53-1.47 (m, 3H), 0.99 (d, J=6.4 Hz, 3H), 0.79 (s, 3H). LCMS: (M+H: 389.1)

Spectra of 48: ¹HNMR: (400 MHz, CDCl₃) δ: 7.13 (t, J=6.4 Hz, 1H), 5.50 (br s, 1H), 5.07 (m, 1H), 4.49 (dd, J=6.0, 10.4 Hz, 1H), 4.28 (dd, J=1.6, 10.4 Hz, 1H), 2.63-2.60 (m, 2H), 2.22 (s, 1H), 2.15-2.13 (m, 3H), 2.05 (br s, 1H), 1.78-1.62 (m, 8H), 1.40-1.37 (m, 1H), 1.03 (d, J=4.8 Hz, 3H), 0.79 (s, 3H). LCMS: (M+H: 389.1).

### Example 49

### DiscoveRx PathHunter® Nrf2-Keap1 Functional Assay

DiscoveRx's Nrf2-Keapl translocation assay was used to profile compounds. Primary Screening was performed in duplicate at a single concentration of 10 μM and EC₅₀ determinations were performed in duplicate at 10 concentrations with 3-fold serial dilutions at a 30 μM top concentration or an otherwise specified top concentration.

The PathHunter® Nuclear Translocation assay detects translocation of a target protein to, or from, the nucleus. In this system, Prolink™ (PK), a small enzyme fragment, is fused to the protein of interest and Enzyme Acceptor (EA) is localized in the nucleus. Activation of the signaling pathway induces the target protein to either transit into the nucleus, thus forcing complementation of the PK and EA fragments, or out of the nucleus, hindering complementation of the fragments.

Assay Protocol:

Cell handling: PathHunter Pathway cell lines were expanded from freezer stocks according to standard procedures. 5000 cells were seeded in Cell Plating Reagent 0 (containing 1% FBS) to a total volume of 20 μL into white walled, 384-well microplates and incubated for the overnight prior to testing.

Agonist format: For Agonist determination, cells were incubated with sample to induce response. Dilution of sample stocks was performed to generate 100× sample in DMSO. Intermediate dilution of sample stocks was performed to generate 5× sample in assay buffer (Cell Plating Reagent 0 containing 1% FBS). 5 μL of 5× sample was added to cells and incubated at room temperature for 6 hours. Vehicle concentration was 1%.

Signal detection: Assay signal was generated through a single addition of 25 μL (100% v/v) of PathHunter Flash Detection reagent, followed by a one hour incubation at room temperature. Microplates were read following signal generation with a PerkinElmer Envision™ instrument for chemiluminescent signal detection.

Data analysis: Compound activity was analyzed using CBIS data analysis suite (ChemInnovation, CA). For agonist mode assays, percentage activity was calculated using the following formula: % Activity=100%×(mean RLU of test sample−mean RLU of vehicle control)/(mean MAX RLU control ligand−mean RLU of vehicle control). For EC₅₀ determination, a control agonist dose response curve was generated in the Keap 1-Nrf2 biosensor assay, data was normalized to the maximal and minimal response observed in the presence of the control ligand and vehicle respectively. CDDO methyl ester was used as a control compound. Compounds were tested in the agonist mode of the assay and data was normalized to the maximal and minimal response observed in the presence of control ligand and vehicle.

TABLE 1
Assay Results:

| | % Activation @ | |
|---|---|---|
| Example # | 10 μM | EC50 (μM) |
| 1 | B | BB |
| 2 | A | AA |
| 3 | A | AA |
| 4 | A | AA |
| 5 | A | AA |
| 6 | B | ND |
| 7 | B | ND |
| 8 | A | BB |
| 9 | A | BB |
| 10 | B | ND |
| 11 | A | BB |
| 12 | B | BB |
| 13 | A | BB |
| 14 | A | CC |
| 15 | B | BB |
| 16 | A | AA |
| 17 | B | BB |
| 18 | C | ND |
| 19 | B | ND |
| 20 | A | BB |
| 21 | B | CC |
| 22 | B | BB |
| 23 | B | ND |
| 24 | B | ND |
| 25 | B | ND |
| 26 | C | ND |
| 27 | C | ND |
| 28 | B | ND |
| 29 | B | ND |
| 30 | B | ND |
| 31 | A | BB |
| 32 | C | ND |
| 33 | C | ND |
| 34 | C | ND |
| 35 | B | CC |
| 36 | B | CC |
| 37 | C | ND |
| 38 | C | ND |
| 39A | A | BB |
| 39B | B | ND |
| 40A | B | ND |
| 40B | A | BB |
| 41A | ND | CC |
| 41B | ND | BB |
| 42A | ND | AA |
| 42B | ND | AA |
| 43A | ND | BB |
| 43B | ND | AA |
| 44A | ND | CC |
| 44B | ND | BB |
| 45A | ND | CC |
| 45B | ND | CC |
| 46A | ND | CC |
| 46B | ND | CC |
| 47 | ND | CC |
| 48 | ND | CC |

In Table 1, % activation: A: >100%; B: 51 to 100%; C: 30 to 50%; D: <30%
EC₅₀: AA: <0.5 μM; BB: 0.5 to 2 μM; CC: >2 μM
nd: not determined

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A compound of formula I 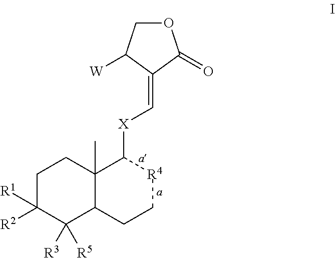
or a single stereoisomer, a mixture of stereoisomers, a racemic mixture of stereoisomers, a solvate, a hydrate, or a pharmaceutically acceptable salt thereof, wherein:
R¹, R², R³ and R⁵ are selected as follows:
i) R¹ and R² are each independently H, C₁₋₆alkyl, OR¹⁰, or NR^{11a}R^{11b}; and R³
and R⁵ are each independently H, hydroxyC₁₋₆alkyl and C₁₋₆alkyl; provided that R¹ and R² are not both OR¹⁰ or NR^{11a}R^{11b} at the same time; or
ii) R² and R³ together with the carbon atoms on which they are substituted form ring b, where ring b is a 4 to 6 membered optionally substituted carbocyclic, heterocyclic or heteroaryl ring; and le and R⁵ are each independently H and C₁₋₆ alkyl;
R¹⁰, R^{11a} and R^{11b} are each independently H, C₁₋₆ alkyl, or 4 to 6 membered optionally substituted carbocyclic or heterocyclyc ring;
R⁴, bond a and bond a′ are selected as follows:
i) R⁴ is CR⁶, bond a is a double bond, and bond a′ is a single bond; or
ii) R⁴ is CR⁶, bond a is a single bond, and bond a′ is a double bond; or
iii) R⁴ is C═CH₂ or CR⁶R⁷, and bonds a and a′ are single bonds;
R⁶ and R⁷ are each independently H or C₁₋₆alkyl; or R⁶ and R⁷ together with the carbon atom on which they are substituted form a 3-6 membered optionally substituted carbocyclic ring;
W is OH or NHR⁹;
R⁹ is C(═O)R¹² or SO₂R^{12a}
R¹² is H, C₁₋₆alkyl or OR^{12a};
R^{12a} is C₁₋₆alkyl; and
X is straight or branched C₁₋₆alkylene, optionally with one or two oxygen atoms in the chain;
where the substituents on the carbocyclic and heterocyclic rings, and on the alkyl groups for R¹, R², R³, R⁵, R⁶, R⁷, R⁹, R¹⁰, R¹¹, R^{11a} and R¹², when present are one to three groups Q¹, where Q¹ is C₁₋₆alkyl, hydroxy, oxo, amino, halo, C₁₋₆alkoxy, hydroxy C₁₋₆alkyl, haloC₁₋₆alkyl, aminoC₁₋₆alkyl, C₁₋₆alkoxy C₁₋₆alkyl, and C₃₋₆cycloalkyl;
and the compound is selected such that
i) when W is OH, R⁴ is C═CH₂ or CH—CH₃, one of le or R² is OH and the other is H, and one of R³ and R⁵ is hydroxymethyl, then the other of R³ or R⁵ is H;
ii) when W is OH, R⁴ is C═CH₂, one of R³ or R⁵ is CH₃ and the other is hydrogen, and one of R¹ and R² is OH, then the other of R¹ or R² is alkyl;
iii) when W is OH, R⁴ is C═CH₂, at least one of le or R² is OH, and one of R³ and R⁵ is aminoalkyl, then the other of R³ or R⁵ is H;
iv) when W is OH, R⁴ is C═CH₂, R¹ and R² are both H, then at least one of R³ and R⁵ is other than methyl;
v) when W is OH, R⁴ is C═CH₂, and R² and R³ together with the carbon atoms on which they are substituted form a 4 membered heterocyclic ring having one oxygen atom; then R¹ is not H;
vi) when W is OH, R⁴ is C—CH₃, and bond ring b is a five membered ring containing two heteroatoms, then at least one heteroatom in ring b is other than nitrogen; and
vii) when W is OH, and ring b is a six membered heterocyclic ring containing two oxygen atoms, then ring b contains at least one additional heteroatom.

2. The compound of claim 1 having formula II: 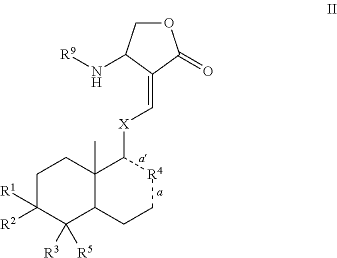
or a single stereoisomer, a mixture of stereoisomers, a racemic mixture of stereoisomers, a solvate, a hydrate, or a pharmaceutically acceptable salt thereof, wherein
R¹, R², R³ and R⁵ are selected as follows:
i) R¹ and R² are each independently H, C₁₋₆alkyl, OR¹⁰, or NR^{11a}R^{11b}; and R³
and R⁵ are each independently H, hydroxyC₁₋₆alkyl and C₁₋₆alkyl; provided that R¹ and R² are not both OR¹⁰ or NR^{11a}R^{11b} at the same time; or
ii) R² and R³ together with the carbon atoms on which they are substituted form ring b, where ring b is a 4 to 6 membered optionally substituted carbocyclic, heterocyclic or heteroaryl ring; and R¹ and R⁵ are each independently H and C₁₋₆ alkyl;
R¹⁰, R^{11a} and R^{11b} are each independently H, C₁₋₆ alkyl, or 4 to 6 membered optionally substituted carbocyclic or heterocyclyc ring;
R⁴, bond a and bond a′ are selected as follows:
i) R⁴ is CR⁶, bond a is a double bond, and bond a′ is a single bond; or
ii) R⁴ is CR⁶, bond a is a single bond, and bond a′ is a double bond; or
iii) R⁴ is C═CH₂ or CR⁶R⁷, and bonds a and a′ are single bonds;
R⁶ and R⁷ are each independently H or C₁₋₆alkyl; or R⁶ and R⁷ together with the carbon atom on which they are substituted form a 3-6 membered optionally substituted carbocyclic ring;
R⁹ is C(═O)R¹² or SO₂R^{12a}
R¹² is H, C₁₋₆alkyl or OR^{12a};
R^{12a} is C₁₋₆alkyl; and
X is straight or branched C₁₋₆alkylene, optionally with one or two oxygen atoms in the chain; and
where the substituents on the carbocyclic and heterocyclic rings, and on the alkyl groups for R¹, R², R³, R⁵, R⁶, R⁷, R⁹, R¹⁰, R¹¹, R^{11a} and R¹², when present are one to three groups Q¹, where Q¹ is C₁₋₆alkyl, hydroxy, oxo, amino, halo, C₁₋₆alkoxy, hydroxy C₁₋₆alkyl, haloC₁₋₆alkyl, aminoC₁₋₆alkyl, C₁₋₆alkoxy C₁₋₆alkyl, and C₃₋₆cycloalkyl.

3. (canceled)

4. The compound of claim 1 having formula III: 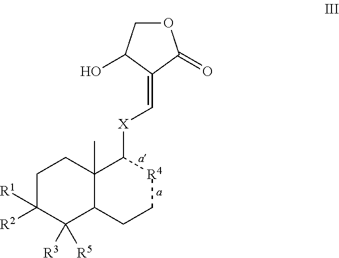
or a single stereoisomer, a mixture of stereoisomers, a racemic mixture of stereoisomers, a solvate, a hydrate, or a pharmaceutically acceptable salt thereof.

5-6. (canceled)

7. The compound of claim 1, wherein R¹, R², R³ and R⁵ are selected as follows:
i) R¹ and R² are each independently H, C₁₋₆alkyl, OR¹⁰, or NR^{11a}R^{11b}; and R³ and R⁵ are each independently H, hydroxyalkyl or C₁₋₆alkyl; provided that R¹ and R² are not both OR¹⁰ or NR^{11a}R^{11b} at the same time; or
ii) R² and R³ together with the carbon atoms on which they are substituted form ring b, where ring b is a 4 to 6 membered carbocyclic, heterocyclic or heteroaryl ring, ring b is optionally substituted with oxo; and R¹ and R⁵ are each independently H and C₁₋₆ alkyl;
R¹⁰ and R^{11a} and R^{11b} are each independently H or C₁₋₆ alkyl;
W is OH or NHC(═O)R¹²;
R¹² is H or C₁₋₆alkyl;
X is straight C₁₋₂alkylene;
R⁴ is C═CH₂ or CR⁶R⁷, bonds a and a′ are single bonds; and R⁶ and R⁷ are each independently H or C₁₋₆alkyl; or R⁶ and R⁷ together with the carbon atom on which they are substituted form a 3-6 membered optionally substituted carbocyclic ring.

8. The compound of claim 1, wherein the compound is of formula V 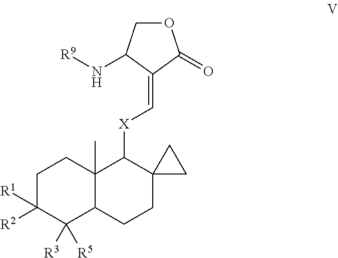
or a single stereoisomer, a mixture of stereoisomers, a racemic mixture of stereoisomers, a solvate, a hydrate, or a pharmaceutically acceptable salt thereof, wherein:
R¹, R², R³ and R⁵ are as follows:
i) R¹, R², R³ and R⁵ are each independently H or C₁₋₆alkyl; or
ii) R² and R³ together with the carbon atoms on which they are substituted form ring b, where ring b is a 4 membered heterocyclic; and R¹ and R⁵ are each independently H or C₁₋₆ alkyl;
R⁹ is C(═O)R¹²;
R¹² is H or C₁₋₆alkyl; and
X is straight C₁₋₂alkylene.

9. The compound of claim 1, wherein the compound is of formula VI 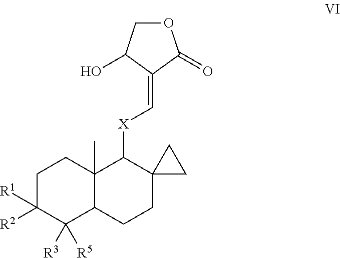
or a single stereoisomer, a mixture of stereoisomers, a racemic mixture of stereoisomers, a solvate, a hydrate, or a pharmaceutically acceptable salt thereof, wherein:
R¹, R², R³ and R⁵ are as follows:
i) R¹, R², R³ and R⁵ are each independently H or C₁₋₆alkyl; or
ii) R² and R³ together with the carbon atoms on which they are substituted form ring b, where ring b is a 4 membered heterocyclic; and R¹ and R⁵ are each independently H or C₁₋₆ alkyl; and
X is straight C₁₋₂alkylene.

10. (canceled)

11. The compound of claim 1, wherein the compound is of formula VIII 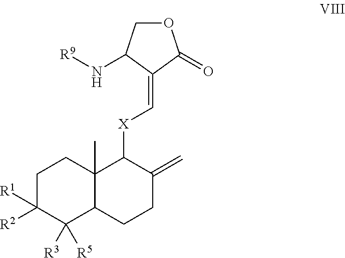
or a single stereoisomer, a mixture of stereoisomers, a racemic mixture of stereoisomers, a solvate, a hydrate, or a pharmaceutically acceptable salt thereof, wherein:
R¹, R², R³ and R⁵ are as follows:
i) R¹, R², R³ and R⁵ are each independently H or C₁₋₆alkyl, or
ii) R² and R³ together with the carbon atoms on which they are substituted form ring b, where ring b is a 4 membered heterocyclic; and R¹ and R⁵ are each independently H and C₁₋₆ alkyl;
R⁹ is C(═O)R¹² or SO₂R^{12a}
R¹² is H, C₁₋₆alkyl or OR^{12a};
R^{12a} is C₁₋₆alkyl; and
X is straight C₁₋₂alkylene.

12. (canceled)

13. The compound of claim 1, wherein the compound is of formula IX 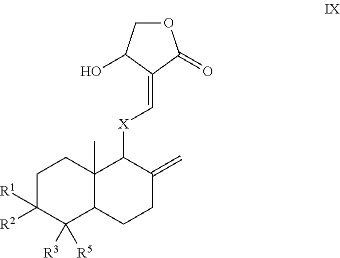
or a single stereoisomer, a mixture of stereoisomers, a racemic mixture of stereoisomers, a solvate, a hydrate, or a pharmaceutically acceptable salt thereof, wherein:
R¹, R², R³ and R⁵ are as follows:
i) R¹ and R² are each independently H, C₁₋₆alkyl, OR¹⁰, or NR^{11a}R^{11b}; and R³ and R⁵ are each independently H and C₁₋₆alkyl; provided that R¹⁰ and R² are not both OR¹⁰ or NR^{11a}R^{11b} at the same time; or
ii) R² and R³ together with the carbon atoms on which they are substituted form ring b, where ring b is a 4 to 6 membered optionally substituted carbocyclic, heterocyclic or heteroaryl ring; and R¹ and R⁵ are each independently H and C₁₋₆ alkyl;
R¹⁰, R^{11a} and R^{11b} are each independently H or C₁₋₆ alkyl; and
X is straight C₁₋₂alkylene, where
i) when one of R¹ or R² is OH and the other is H, and one of R³ and R⁵ is hydroxymethyl, then the other of R³ or R⁵ is H;
ii) when one of R³ or R⁵ is CH₃ and the other is hydrogen, and one of R¹ and R² is OH, then the other of R¹ or R² is alkyl;
iii) when at least one of R¹ or R² is OH, and one of R³ and R⁵ is aminoalkyl, then the other of R³ or R⁵ is H;
iv) when R¹ and R² are both H, then at least one of R³ and R⁵ is other than methyl; and
v) when R² and R³ together with the carbon atoms on which they are substituted form a 4 membered heterocyclic ring having one oxygen atom; then R¹ is not H.

14. The compound of claim 1, wherein the compound is of formula X 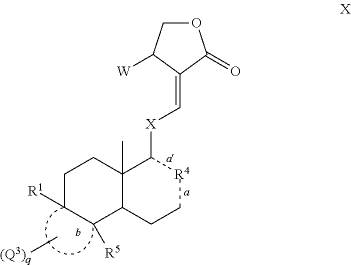
or a single stereoisomer, a mixture of stereoisomers, a racemic mixture of stereoisomers, a solvate, a hydrate, or a pharmaceutically acceptable salt thereof, wherein:
R¹ is H or C₁₋₆alkyl;
R⁵ is H or C₁₋₆ alkyl;
ring b is a carbocyclic or heterocyclic 4-6 membered ring;
Q³ is oxo;
q is 0-1;
R⁴, bond a and bond a′ are as follows:
i) R⁴ is CR⁶, bond a is a double bond, and bond a′ is a single bond; or
ii) R⁴ is CR⁶, bond a is a single bond, and bond a′ is a double bond; or
iii) R⁴ is C═CH₂ or CR⁶R⁷, and bonds a and a′ are single bonds; R⁶ and R⁷ are each independently H or C₁₋₆alkyl; or R⁶ and R⁷ together with the carbon atom on which they are substituted form a 3-6 membered optionally substituted carbocyclic ring;
W is OH or NHR⁹;
R⁹ is C(═O)R¹² or SO₂R^{12a}
R¹² is H, C₁₋₆alkyl or OR^{12a};
R^{12a} is C₁₋₆alkyl; and
X is straight or branched C₁₋₆alkylene, optionally with one or two oxygen atoms in the chain;
where the substituents on the carbocyclic and heterocyclic rings and on the alkyl groups for R¹, R⁵, R⁹, R¹⁰, R¹¹, R^{11a}, R¹² and R^{12a}, when present are one to three groups Q¹, where Q¹ is C₁₋₆alkyl, hydroxy, amino, halo, C₁₋₆alkoxy, hydroxy C₁₋₆alkyl haloC₁₋₆alkyl aminoC₁₋₆alkyl, C₁₋₆alkoxy C₁₋₆alkyl, and C₃₋₆cycloalkyl, where
i) when W is OH, R⁴ is C—CH₃, and bond ring b is a five membered ring containing two heteroatoms, then at least one heteroatom in ring b is other than nitrogen; and
ii) when W is OH and ring b is a six membered heterocyclic ring containing two oxygen atoms, then ring b contains at least one additional heteroatom.

15. The compound of claim 1, wherein the compound is of formula XI 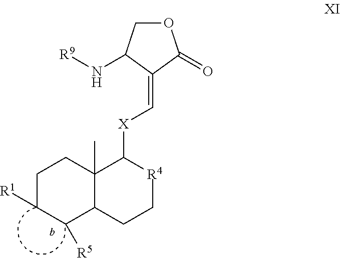
or a single stereoisomer, a mixture of stereoisomers, a racemic mixture of stereoisomers, a solvate, a hydrate, or a pharmaceutically acceptable salt thereof, wherein:
R¹ is H or C₁₋₆alkyl;
R⁵ is H or C₁₋₆ alkyl;
ring b is a carbocyclic or heterocyclic 4-6 membered ring;
R⁴ is C═CH₂ or CR⁶R⁷,
R⁶ and R⁷ together with the carbon atom on which they are substituted form a 3-6 membered carbocyclic ring;
R⁹ is C(═O)R¹² or SO₂R^{12a}
R¹² is H, C₁₋₆alkyl or OR^{12a};
R^{12a} is C₁₋₆alkyl; and
X is straight or branched C₁₋₆alkylene.

16. The compound of claim 1, wherein the compound is of formula XII 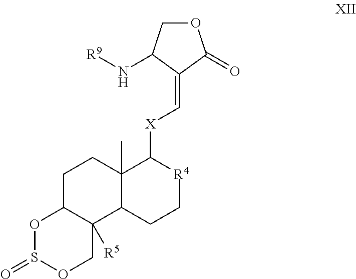
or a single stereoisomer, a mixture of stereoisomers, a racemic mixture of stereoisomers, a solvate, a hydrate, or a pharmaceutically acceptable salt thereof, wherein:
R⁵ is H or C₁₋₆ alkyl;
R⁴ is C═CH₂ or CR⁶R⁷,
R⁶ and R⁷ together with the carbon atom on which they are substituted form a 3-6 membered carbocyclic ring;
R⁹ is C(═O)R¹² or SO₂R^{12a}
R¹² is H, C₁₋₆alkyl or OR^{12a};
R^{12a} is C₁₋₆alkyl; and
X is straight or branched C₁₋₆alkylene.

17. The compound of claim 1, wherein the compound is of formula XIII 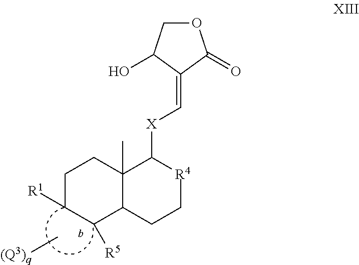
or a single stereoisomer, a mixture of stereoisomers, a racemic mixture of stereoisomers, a solvate, a hydrate, or a pharmaceutically acceptable salt thereof, wherein:
R¹ is H or C₁₋₆alkyl;
R⁵ is H or C₁₋₆ alkyl;
ring b is a carbocyclic or heterocyclic 4-6 membered ring;
Q³ is oxo;
q is 0-1;
R⁴ is C═CH₂ or CR⁶R⁷;
R⁶ and R⁷ together with the carbon atom on which they are substituted form a 3-6 membered optionally substituted carbocyclic ring; and
X is straight or branched C₁₋₆alkylene, where
i) when R⁴ is C—CH₃, and bond ring b is a five membered ring containing two heteroatoms, then at least one heteroatom in ring b is other than nitrogen; and
ii) when and ring b is a six membered heterocyclic ring containing two oxygen atoms, then ring b contains at least one additional heteroatom.

18. The compound of claim 1, wherein the compound is of formula XIV 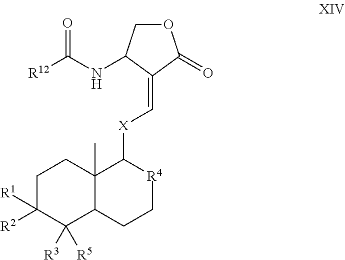
or a single stereoisomer, a mixture of stereoisomers, a racemic mixture of stereoisomers, a solvate, a hydrate, or a pharmaceutically acceptable salt thereof, wherein:
R¹, R², R³ and R⁵ are as follows:
i) R¹ and R² are each independently H, C₁₋₆alkyl, OR¹⁰, or NR^{11a}R^{11b}; and R³ and R⁵ are each independently H, hydroxyC₁₋₆alkyl or C₁₋₆alkyl; provided that R¹ and R² are not both OR¹⁰ or NR^{11a}R^{11b} at the same time; or
ii) R² and R³ together with the carbon atoms on which they are substituted form ring b, where ring b is a 4 to 6 membered carbocyclic, heterocyclic or heteroaryl ring; ring b is optionally substituted with an oxo group, and R¹ and R⁵ are each independently H and C₁₋₆ alkyl;
R¹⁰ and R^{11a} and R^{11b} are each independently H, C₁₋₆ alkyl, or 4 to 6 membered carbocyclic or heterocyclyc ring;
R⁴ is C═CH₂ or CR⁶R⁷;
R⁶ and R⁷ together with the carbon atom on which they are substituted form a 3-6 membered carbocyclic ring;
R¹² is H or C₁₋₆ alkyl; and
X is straight or branched C₁₋₆alkylene.

19. The compound of claim 1, wherein the compound is of formula XV 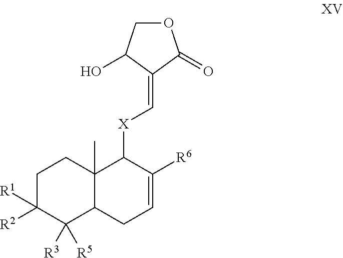
or a single stereoisomer, a mixture of stereoisomers, a racemic mixture of stereoisomers, a solvate, a hydrate, or a pharmaceutically acceptable salt thereof, wherein:
R¹, R², R³ and R⁵ are selected as follows:
i) R¹ and R² are each independently H, C₁₋₆alkyl, OR¹⁰, or NR^{11a}R^{11b}; and R³ and R⁵ are each independently H or C₁₋₆alkyl; provided that le and R² are not both OR¹⁰ or NR^{11a}R^{11b} at the same time; or
ii) R² and R³ together with the carbon atoms on which they are substituted form ring b, where ring b is a 4 to 6 membered optionally substituted carbocyclic, heterocyclic or heteroaryl ring; and R¹ and R⁵ are each independently H or C₁₋₆ alkyl;
R¹⁰, R^{11a} and R^{11b} are each independently H, C₁₋₆ alkyl, 4 to 6 membered optionally substituted carbocyclic or heterocyclyc ring;
R⁶ is H or C₁₋₆alkyl; and
X is straight or branched C₁₋₆alkylene, optionally with one or two oxygen atoms in the chain;
where the substituents on the carbocyclic and heterocyclic rings and on the alkyl groups for R¹, R², R³, R⁵, R¹⁰, R¹¹ and R^{11a} when present are one to three groups Q¹, where Q¹ is C₁₋₆alkyl, hydroxy, amino, halo, C₁₋₆alkoxy, hydroxy C₁₋₆alkyl, haloC₁₋₆alkyl, aminoC₁₋₆alkyl, C₁₋₆alkoxy C₁₋₆alkyl, or C₃₋₆cycloalkyl.

20. (canceled)

21. The compound of claim 1, wherein the compound is of formula XXI 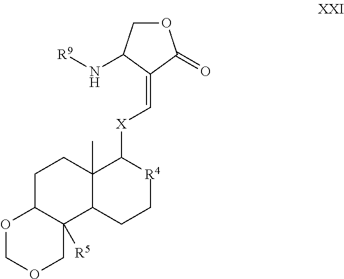
or a single stereoisomer, a mixture of stereoisomers, a racemic mixture of stereoisomers, a solvate, a hydrate, or a pharmaceutically acceptable salt thereof, wherein:
R⁵ is H or C₁₋₆ alkyl;
R⁴ is C═CH₂ or CR⁶R⁷,
R⁶ and R⁷ together with the carbon atom on which they are substituted form a 3-6 membered carbocyclic ring;
R⁹ is C(═O)R¹² or SO₂R^{12a};
R¹² is H, C₁₋₆alkyl, C₁₋₆alkoxy C₁₋₆alkyl or OR^{12a};
R^{12a} is C₁₋₆alkyl; and
X is straight or branched C₁₋₆alkylene.

22. The compound claim 1, wherein the compound is selected from the following:

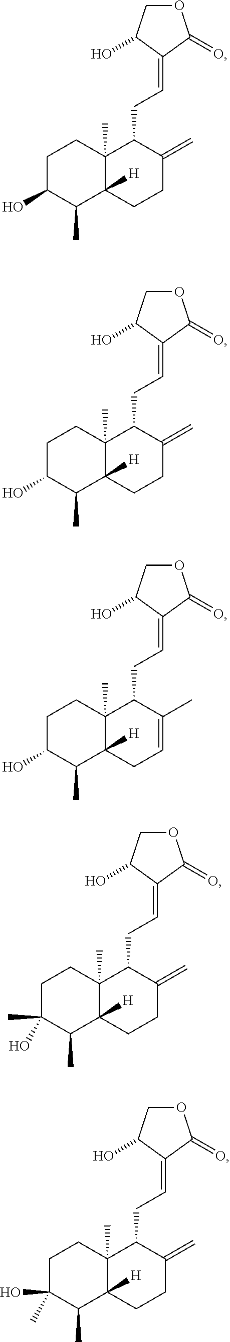

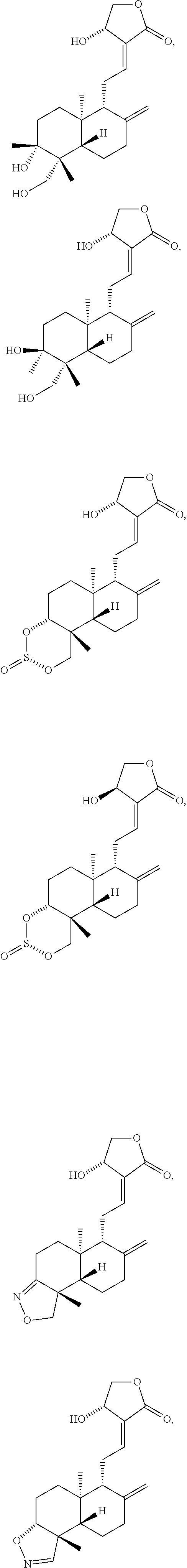

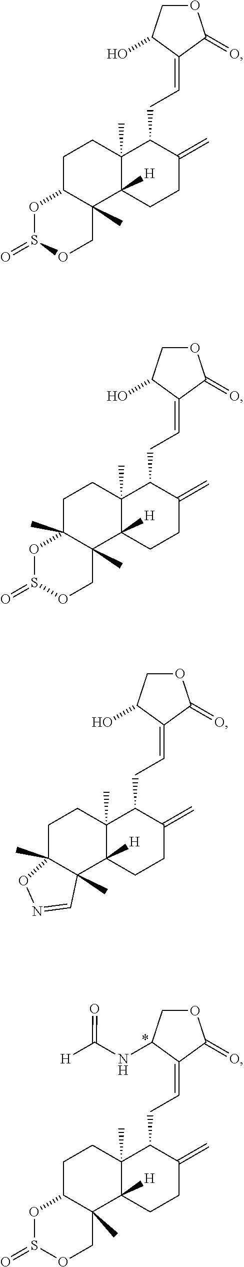

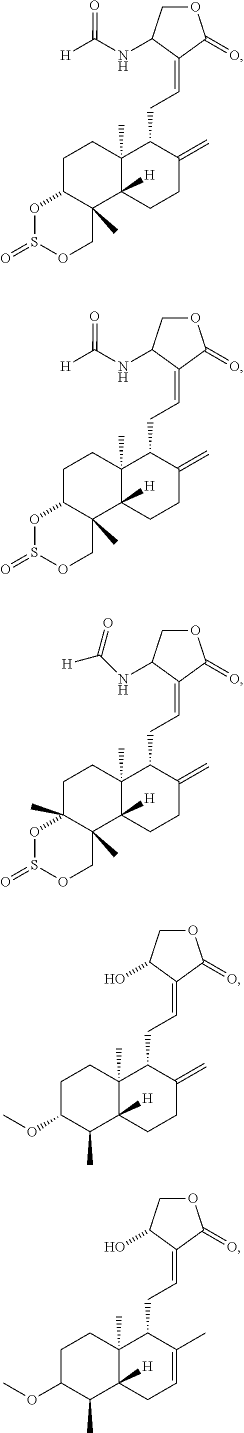

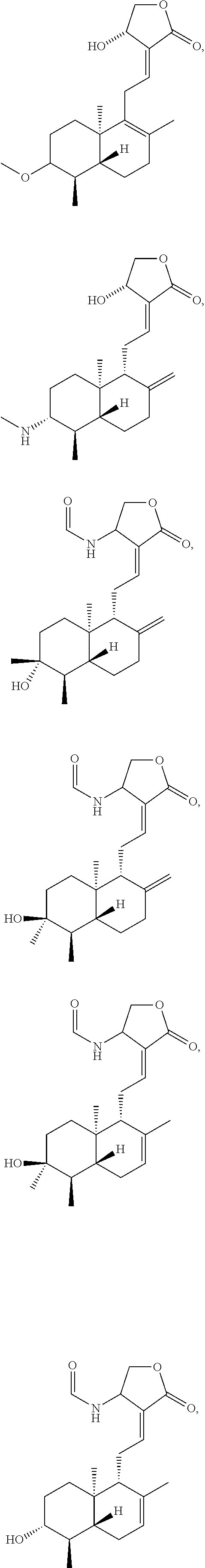

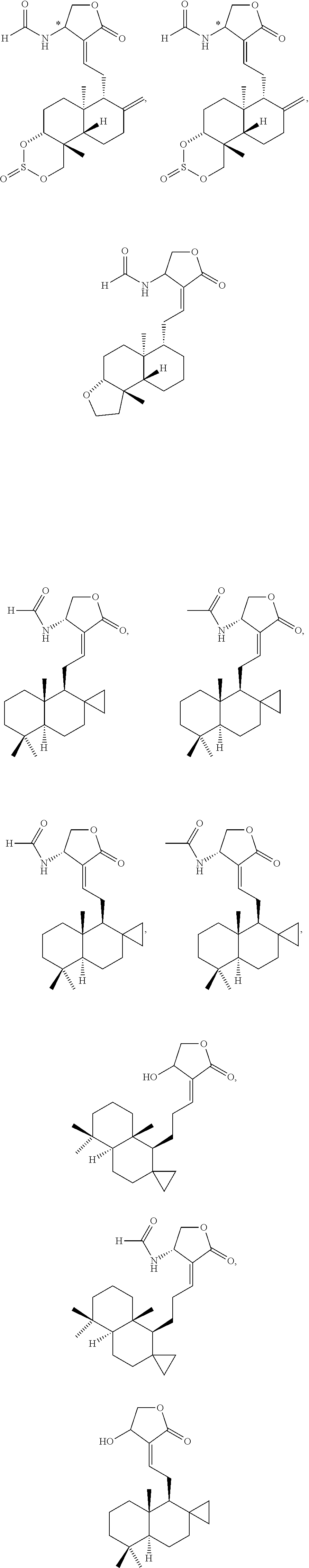

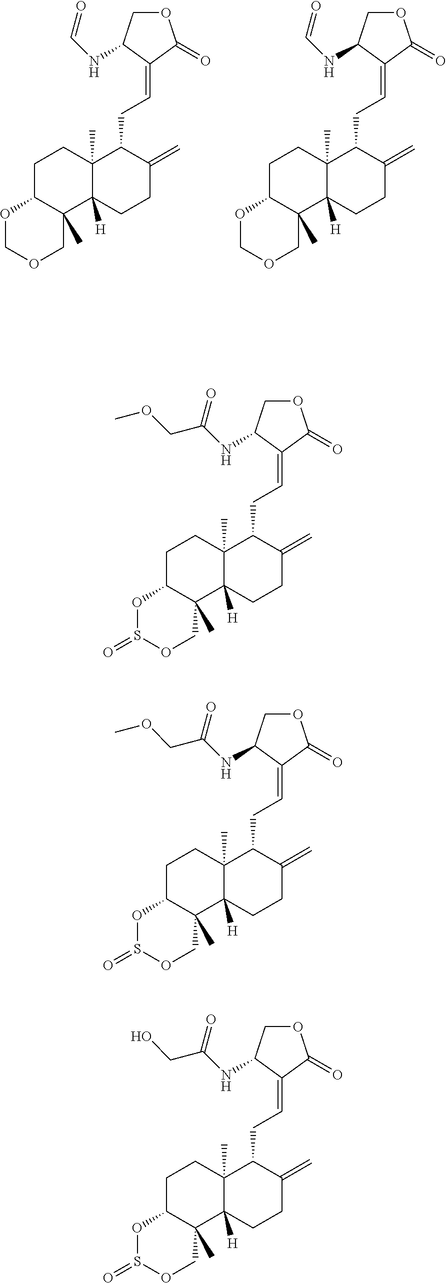

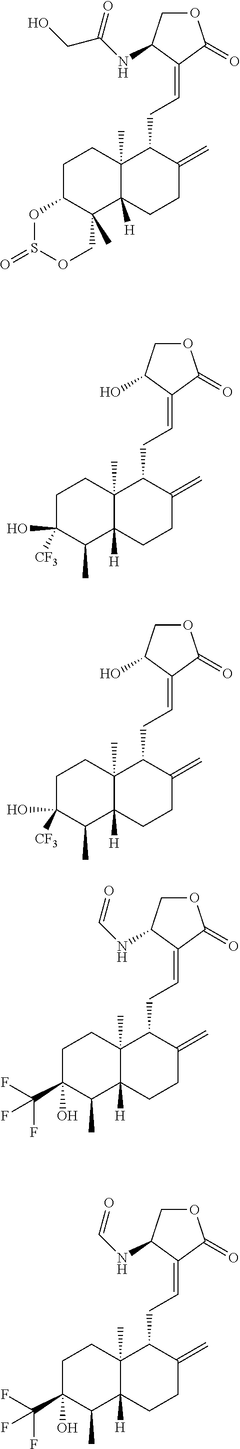

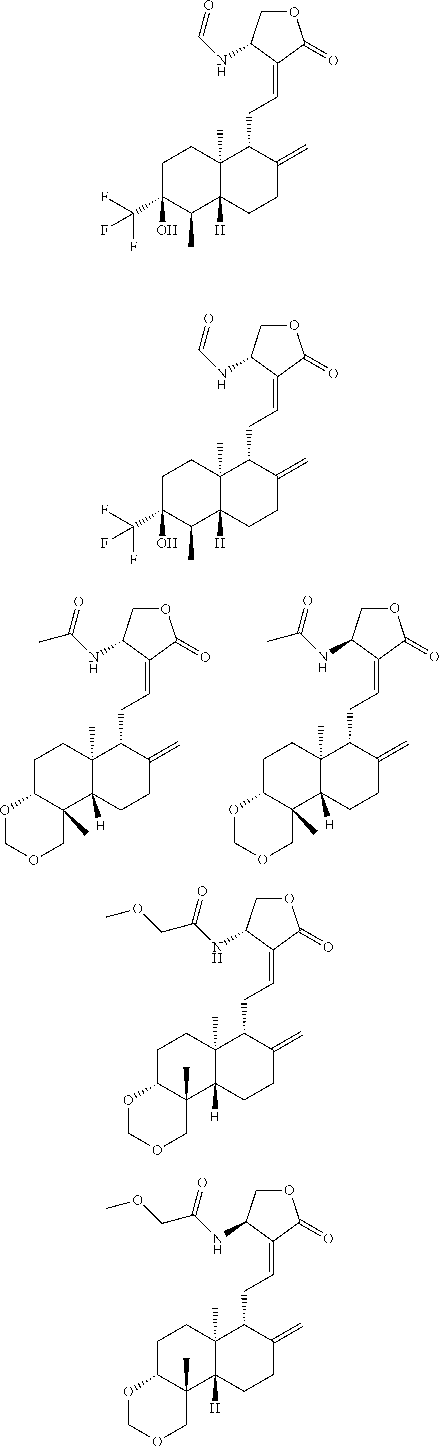

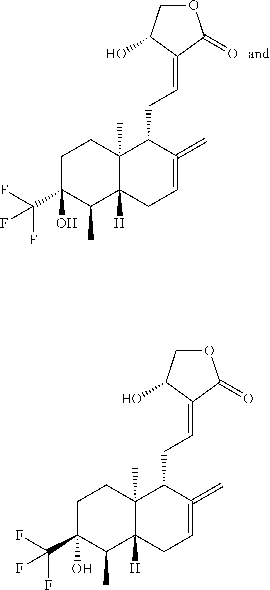

23. A pharmaceutical composition comprising a compound of claim 1 and a pharmaceutically acceptable carrier.

24. A method of activating the Nrf2 pathway comprising contacting cells that express an Nrf2 receptor with a sufficient amount of a compound of claim 1.

25. A method of treating or prophylaxis of one or more neurodegenerative diseases, comprising administering to a subject in need of treatment for the neurodegenerative disease an effective amount of a compound of claim 1.

26-27. (canceled)

28. A method for the treatment or prophylactic treatment of one or more diseases or disorders in a subject in need thereof, wherein the method comprises administering to the subject an effective amount of a compound of claim 1, wherein the disease or disorder is one or more of multiple sclerosis, amyotrophic lateral sclerosis, Alzheimer's disease, Parkinson's disease and Huntington's disease, acute haemorrhagic leucoencephalomyelitis, Hurst's disease, encephalomyelitis, optic neuritis, spinal cord lesions, acute necrotizing myelitis, transverse myelitis, chronic progressive myelopathy, progressive multifocal leukoencephalopathy, radiation myelopathy, HTLV-1 associated myelopathy, monophasic isolated demyelination, central pontine myelinolysis, leucodystrophy, inflammatory demyelinising polyneuropathy, acute Guillain-Barre syndrome, polyneuritis, myasthenia gravis, Eaton Lambert Syndrome, encephalomyelitis, inflammatory bowel disease, Crohn's disease, lupus, systemic Lupus erythematodes, asthma, Leber's disease, Devic's disease, Friedrich's Ataxia, mitochondrial Central Nervous System diseases, scleroderma, uveitis, anti-phospholipid antibody syndrome, polyarthritis, polyarticular juvenile idiopathic arthritis, sickle cell disease, ankylosing spondylitis, myositis, atherosclerosis, diabetic peripheral neuropathy, head injury, stroke, HIV-dementia, myocardial infarction, angina pectoris, cardiac insufficiency, psoriasis, psoriatic arthritis, Sjogren's syndrome, diabetes, blistering skin diseases, sarcoidosis, osteoarthritis, ulcerative colitis, vasculitis and lung fibrosis, idiopathic pulmonary fibrosis, liver fibrosis, kidney fibrosis, acute kidney injury, chronic kidney disease—diabetic nephrophathy, graft-versus-host reactions, Hashimoto's thyroiditis, Grave's disease, pernicious anaemia, hepatitis, neurodermatitis, retinopathia pigmentosa, forms of mitochondrial encephalomyopathy, osteochondritis syphilitica , cutis marmorata, Behcet disease, panarteriitis, osteoarthritis, gout, artenosclerosis, Reiter's disease, pulmonary granulomatosis, types of encephalitis, endotoxic shock, sepsis, pneumonia, anorexia nervosa, Rennert T-lymphomatosis, mesangial nephritis, post-angioplastic restenosis, reperfusion syndrome, cytomegaloviral retinopathy, adenoviral diseases, AIDS, post-herpetic or post-zoster neuralgia, mononeuropathia multiplex, mucoviscidosis, Bechterew's disease, Barett oesophagus, Epstein-Barr virus infection, cardiac remodeling, interstitial cystitis, human tumour radiosensitisation, multi-resistance of malignant cells to chemotherapeutic agents, granuloma annulare, cancers, chronic obstructive pulmonary diseases, PDGF induced thymidine uptake of bronchial smooth muscle cells, bronchial smooth muscle cell proliferation, Adrenal Leukodystrophy, Alcoholism, Alper's disease, Ataxia telangiectasia, Batten disease, Bovine spongiform encephalopathy, Cerebral palsy, Cockayne syndrome, Corticobasal degeneration, Creutzfeldt-Jakob disease, Familial Fatal Insomnia, Frontotemporal lobar degeneration, Kennedy's disease, Lewy body dementia, Neuroborreliosis, Machado-Joseph disease, Multiple System Atrophy, Narcolepsy, Niemann Pick disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spinocerebellar ataxia, Spinal muscular atrophy, Steele-Richardson-Olszewski disease, Tabes dorsalis, Toxic encephalopathy, Mitochondrial Encephalomyopathy; Lactic Acidosis; Stroke, Myoclonic Epilepsy; Ragged Red Fibers, Progressive External Opthalmoplegia, Leigh's Syndrome, Myopathy and external ophthalmoplegia; Neuropathy; Gastro-Intestinal; Encephalopathy, Kearns-Sayre Syndrome, NARP, Hereditary Spastic Paraparesis, Mitochondrial myopathy,optic neuritis, progressive multifocal leucoencephalopathy, Pyoderma Gangrenosum, Erosive Pustular Dermatosis of the Scalp, Sweet's Syndrome, Bowel-associated Dermatosis-arthritis Syndrome, Pustular Psoriasis, Acute Generalized Exanthematous Pustulosis, Keratoderma Blenorrhagicum, Sneddon-Wilkinson Disease, Amicrobial Pustulosis of the Folds, Infantile Acropustulosis, Transient Neonatal Pustulosis, Neutrophilic Eccrine Hidradenitis, Rheumatoid Neutrophilic Dermatitis, Neutrophilic Urticaria, Still's Disease, Erythema Marginatum, Unclassified Periodic Fever Syndromes/Autoinflammatory Syndromes, Bullous Systemic Lupus Erythematosus, Neutrophilic Dermatosis of the Dorsal Hands, anaphylaxis, allergic rhinitis, allergic asthma, lung cancer, severe asphyxic episodes of asthma, acute lung injury, Acute Respiratory Distress Syndrome, ischemia reperfusion injury, septicemia with multiorgan failure, inderteminate colitis, sickle cell crisis, or acute chest syndrome, Scleroderma lung disease, Chronic Asthma, Radiation-Induced Fibrosis Sarcoidosis, Pulmonary Hypertension, Bronchopulmonary Dysplasia, Lung Transplant Rejection, Pulmonary GVHD Complications, Interstitial pneumonia Syndrome in transplant recipients, COPD, Silicosis, Asbestosis, Primary sclerosing cholangitis, Alcohol-induced hepatic fibrosis, Autoimmune hepatitis, Chronic viral hepatitis, Primary biliary cirrhosis, Non-alcohol Steatohepatitis, Liver transplant rejection, Hepatic complications of GVHD, Veno-occlusive disease in transplant recipients, Focal Segmental Glomerular Sclerosis, Diabetic nephropathy, IgA nephropathy, Renal complications of GVHD, Acute renal failure post CABG, Lupus nephritis, Hypertension-induced Renal Fibrosis, HIV-associated nephropathy, Peritoneal dialysis-induced peritoneal fibrosis, Retroperitoneal fibrosis, Idiopathic Glomerulosclerosis, Kidney transplant rejection, Alport syndrome, Restenosis, Subarachnoid hemorrhage, Heart transplant rejection, Cosmetic surgery, Chronic wounds, Burns, Surgical adhesions, Keloids, Donor graft re-epithelialization, Myelofibrosis, Corneal transplant, LASIX, Trabeculectomy, Systemic sclerosis, Radiation induced fibrosis, Peripatellar Fibrosis, and Dupuytren's Contractures.

29-30. (canceled)

31. The method of claim 24, wherein the method comprises administering a second therapeutic agent.
